(19) United States
(12) Patent Application Publication
(10) Pub. No.: US 2011/0065859 A1
(43) Pub. Date: Mar. 17, 2011

(54) THERMALLY STABLE ALIGNMENT MATERIALS

(75) Inventors: Izabela Bury, Basel (CH); Guy Marck, Schilerbach (FR); Satish Palika, Basel (CH); Peggy Studer, Huningue (FR)

(73) Assignee: ROLIC AG, Zug (CH)

(21) Appl. No.: 12/596,571

(22) PCT Filed: Apr. 10, 2008
(86) PCT No.: PCT/EP2008/002835

§ 371 Date: Oct. 19, 2009

(30) Foreign Application Priority Data
May 2, 2007 (EP) 07107301.9
May 31, 2007 (EP) 07109358.7
Oct. 16, 2007 (EP) 07118631.6

(51) Int. Cl. C08L 31/06; C07C 69/017; C08F 122/10

(52) U.S. Cl. 524/555; 560/65; 560/44; 526/321; 526/312; 524/558

(57) ABSTRACT

A thermally stable diamine compound of formula (I′) is proposed as well as polymers, copolymers, polyamic acids, polyamic acid esters, or polyimides based on such compound.

The invention relates to thermally stable alignment materials comprising diamine compounds of formula (I′), and in addition relates to oligomers, polymers and copolymers from the class of polyamic acids, polyamic acid esters or polyimides (and any mixtures thereof) obtained by the reaction of a diamine compound represented by the general formula (I′) and optionally of one or more additional other diamines, with one or more tetracarboxylic acid anhydrides, and to the use of these diamine compounds, oligomers, polymers and copolymers for the preparation of orientation layers for liquid crystals and in the construction of unstructured and structured optical elements and multi-layer systems.

Methods for the preparation of orientation layers for liquid crystal materials are well known to the skilled person. Customarily used are uniaxially rubbed polymer orientation layers, such as for example polyimides. In addition, orientation layers are obtained using irradiation technique with aligning light as for example described in Jpn. J. Appl. Phys., 31 (1992), 2155-64 (Schadt et al).

In the manufacturing of displays or films the decomposition of alignment materials is of great concern due to the contamination of the production lines, any part of the device, display or equipment, as well as the areas of the substrates which are not coated with the alignment material, which are “uncoated areas” of the orientation layer. The surface properties, such as surface energies, of the uncoated areas will be changed by this contamination due to the absorption of decomposition compounds of the alignment material e.g. volatile fragments of the alignment material, which could have detrimental effects on subsequent coatings. Thus, the wetting and/or adhesion properties of coatings or liquids subsequently applied on these “uncoated areas” would be changed which would lead to defects (e.g. adhesion failure). It is well known that wetting and good adhesion are favoured when the substrate's critical surface tension is high and the surface tension of the coating/adhesive is low: hence, failures or defects might arise if the difference in the surface tension between the coating formulation to be applied and the surface energy of the “uncoated areas” are not respecting this basic rule. Modification, in particular decrease of the surface energy, will be particularly dramatic in the case if fluorinated fragments are generated during the baking process of the alignment layer.

In order to avoid these adverse effects during the manufacturing process of displays or films, it was the aim of the present invention to provide alignment material, which is thermically stable under the given process temperature.

Thus, the present invention relates to the use of thermally stable alignment materials for the preparation of orientation layers for liquid crystals comprising diamine compounds of formula (I′),

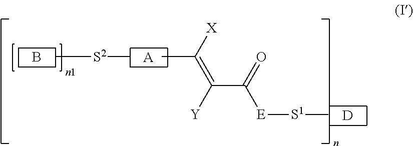

wherein,
A represents an unsubstituted or substituted carbocyclic or heterocyclic aromatic group selected from a monocyclic ring of five or six atoms, two adjacent monocyclic rings of five or six atoms, a bicyclic ring system of eight, nine or ten atoms, or a tricyclic ring system of thirteen or fourteen atoms; and
   wherein
B represents a straight-chain or branched C₁-C₁₆alkyl group, which is unsubstituted or substituted by
   di-(C₁-C₁₆alkyl)amino, C₁-C₆alkyloxy, nitro, cyano, and/or halogene, such as fluorine, chlorine, bromine or jodine; and wherein one or more —CH₂— group may independently from each other be replaced by a linking group;
D is a selected from the group of the following compounds:

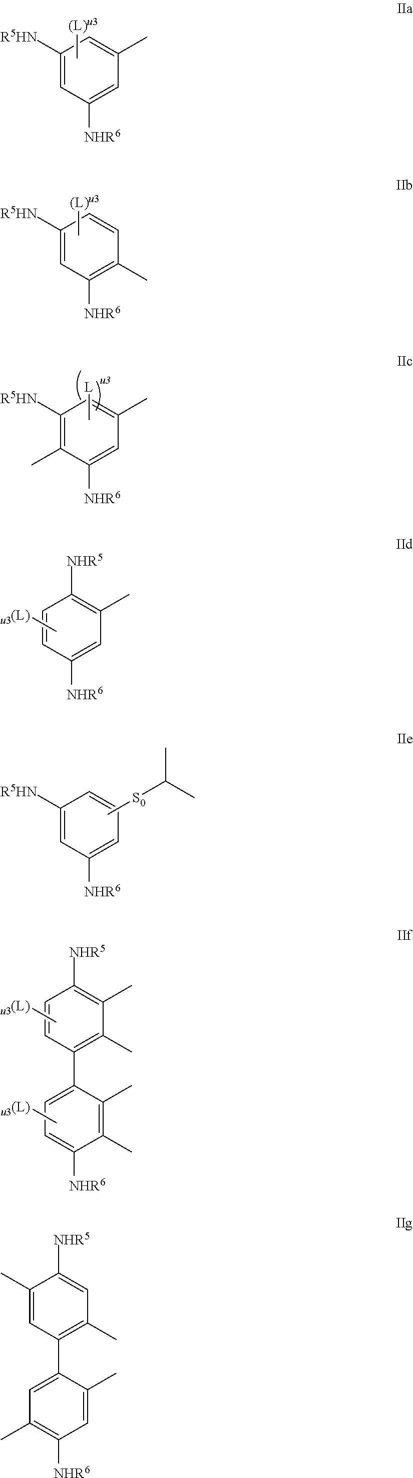

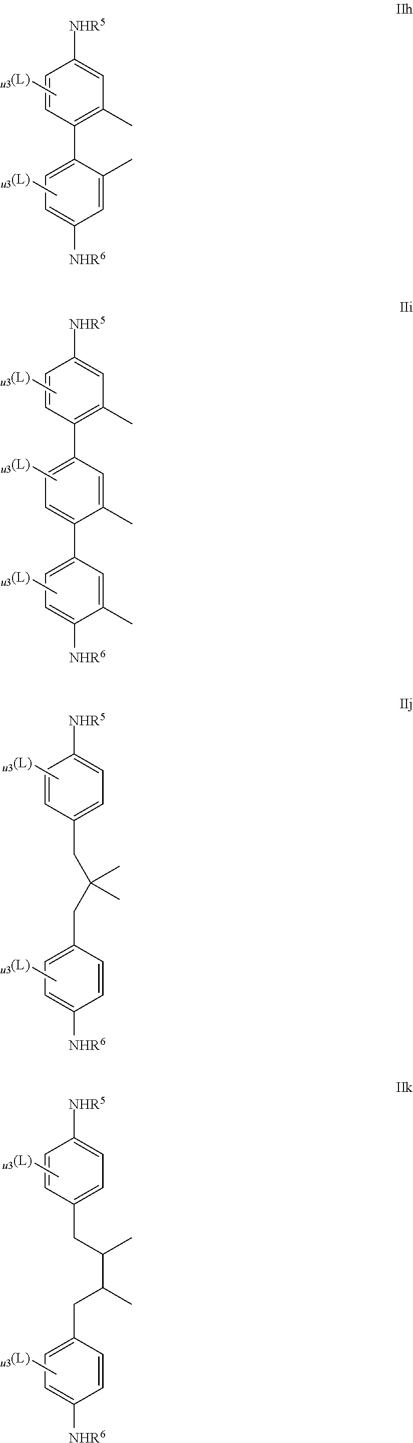

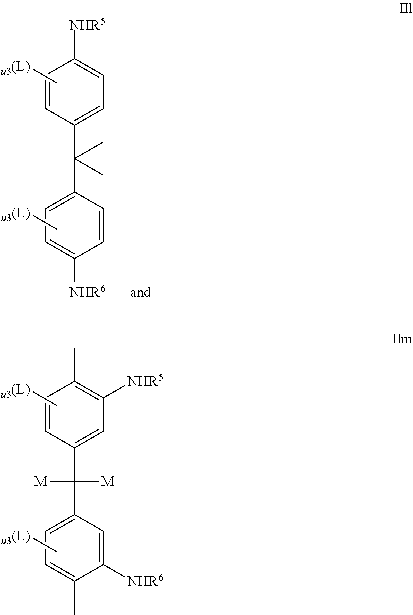

“-” denotes the linking(s) of D to S¹ in compound (I) and represents a single bond; and
S⁰ represents a single bond or a spacer unit, which is a straight-chain or branched, substituted or unsubstituted C₁-C₂₄alkylen, preferably C₁-C₆alkylen, wherein one or more, preferably non-adjacent, —CH₂— group may be replaced by a linking group, preferably replaced by —O—, —O(CO)—, —(CO)O—, —NR₁CO—, —CONR₁—, wherein R₁ is hydrogen or C₁-C₆alkyl; most preferably S⁰ is a single bond;
M represents a H, C₁-C₂₄alkyl and CF₃;
L is —CH₃, —COCH₃, —OCH₃, nitro, cyano, halogen, CH₂═CH—, CH₂═C(CH₃)—, CH₂═CH—(CO)O—, CH₂═CH—O—, —NR⁵R⁶, CH₂═C(CH₃)—(CO)O— or CH₂═C(CH₃)—O—,
   wherein:
R⁵, R⁶ each independently from each other represents a hydrogen atom or C₁-C₆alkyl and/or an alicyclic group;
u₃ is an integer from 0 to 2, preferably D is selected from the group of the following compounds IIa, IIb and IIj, more preferably D is selected from the following compounds IIa and Mb;
E represents an aromatic group, an oxygen atom, a sulphur atom, —NH—, —N(C₁-C₆alkyl)-, —CR²R³,
   wherein R² and R³ are independently from each other hydrogen or a cyclic, straight-chain or branched, substituted or unsubstituted C₁-C₂₄alkyl, wherein one or more —CH₂— group(s) may be independently from each other replaced by a linking group, and with the proviso that at least one of R² and R³ is not hydrogen;
S¹ represents a single bond or a cyclic, straight-chain or branched, substituted or unsubstituted C₁-C₂₄alkylen, if D is a compound of formulae IIa, IIc, IId, IIe, IIf, IIg, IIh, IIi, IIj, IIk, preferably D is a compound of formulae IIa, IIj and IIk; and if D is a compound of formula IIb, S¹ represents a single bond or a cyclic, straight-chain or branched, substituted or unsubstituted C₂-C₂₄alkylen, preferably C₅-C₂₄alkylen, more preferably C₁₀-C₂₄alkylen;
S² represents a spacer unit;
X, Y each independently from each other represents hydrogen, fluorine, chlorine, cyano, unsubstituted or with fluorine substituted C₁-C₁₂alkyl, in which one or more —CH₂— groups may be replaced by a linking group;
n, n1 each independently from each other represents 1, 2, 3 or 4, preferably n1 is 1 and n is 1 or 2;
   with the proviso that if n is 2, 3, or 4, each A, B, x₁, E, S¹, S², X, Y are identical or different; and if n1 is 2, 3 or 4 each B, x₁ is identical or different;
   preferably,
   wherein, if n>1, compound (I) has several side-chains [wherein side-chain has the meaning of structures (I) without the group D], which are linked to residue D at one atomic position within group D, e.g. two or three side chains linked to one single carbon atom within group D, or they can be linked to group D at different atomic positions within group D, e.g. at adjacent atomic positions within group D, or/and they can linked spaced further apart.

In the context of the present invention “Thermally stable” means that the surface of the uncoated areas is not contaminated during the thermal baking at the given process temperature, which is preferably >150° C., more preferably >180° C. and most preferably >than 200° C.

A preferred embodiment of the present invention relates to the use of the invention, wherein the orientation layer comprises coated and uncoated areas, whereby the surface of the uncoated areas are not contaminated during the thermal baking in the process of preparation of an orientation layer.

The uncoated and uncontaminated area represents the substrate, or any part of the production lines, of the device, the display or the equipment, as well as the areas of the substrates which are not coated with the alignment material, which are “uncoated areas” of the orientation layer.

In the context of the present invention substrate represents a support, whereon the orientation layer is coated or printed. Suitable materials are for example glass or plastic substrates, optionally coated with indium tin oxide (ITO) are used.

The surface is for example characterised by surface energy and the chemical composition.

If the surface is contaminated the surface energy changes, preferably decreases.

A further preferred embodiment of the present invention relates to the use of the invention, wherein the uncoated areas have a surface energy of >40 mN/Meter, preferably >50 mN/Meter, and more preferably >55 mN/Meter, on ITO coated glass plates.

Preferably, the present invention relates to diamine compound of formula (I):

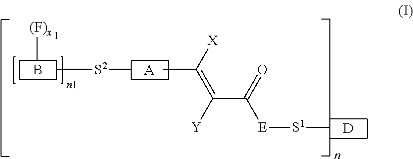

wherein,
A represents an unsubstituted or substituted carbocyclic or heterocyclic aromatic group selected from a monocyclic ring of five or six atoms, two adjacent monocyclic rings of five or six atoms, a bicyclic ring system of eight, nine or ten atoms, or a tricyclic ring system of thirteen or fourteen atoms;
   and
   wherein the following compound residue of formula (I), the compound residue (Ia)

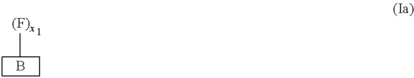

represents a straight-chain or branched C₁-C₁₆fluoralkyl group, wherein
F is fluorine, and
x₁ is an integer from 0 to 15, preferably an integer from 0 to 10; more preferably 0, 1, 2, 3, 4, 5, 6, 7, 8 or 9 and most preferred 0 or 3, 4, 5 or 7;
B represents a straight-chain or branched C₁-C₁₆alkyl group, which is in addition to its fluorine substituent(s) unsubstituted or substituted by di-(C₁-C₁₆alkyl)amino, C₁-C₆alkyloxy, nitro, cyano and/or chlorine; and wherein one or more —CH₂— group may independently from each other be replaced by a linking group;
D is a selected from the group of the following compounds:

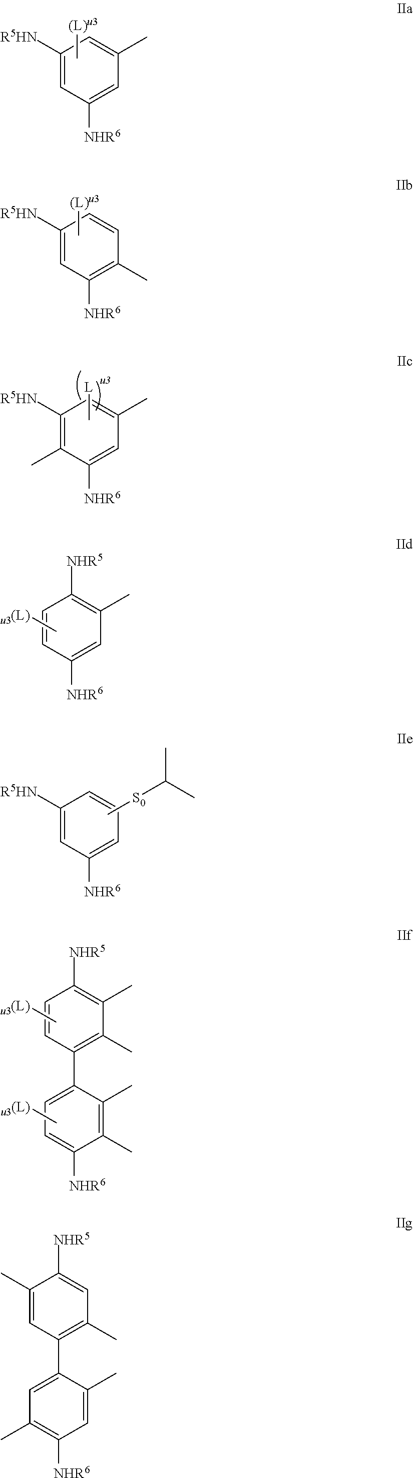

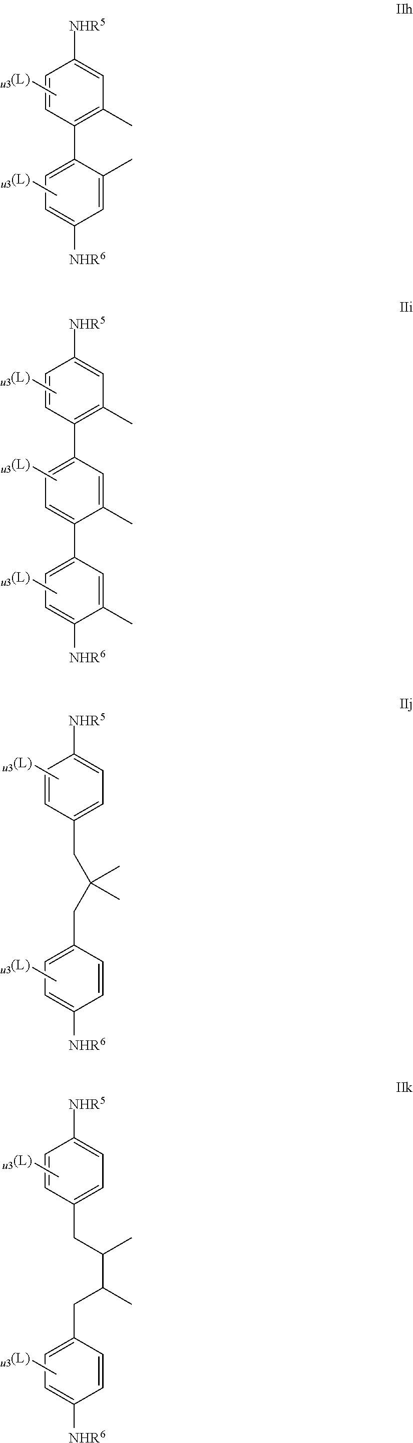

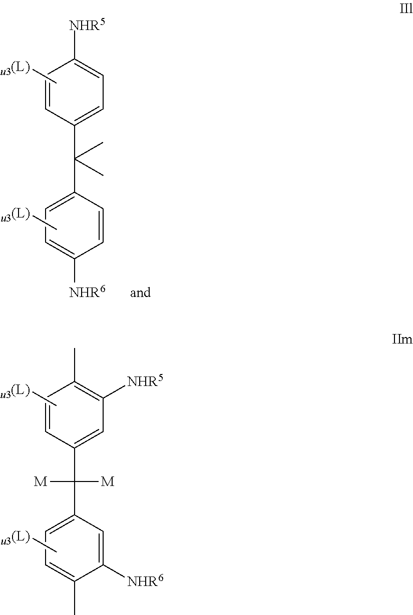

“-” denotes the linking(s) of D to S¹ in compound (I) and represents a single bond; and
S⁰ represents a single bond or a spacer unit, which is a straight-chain or branched, substituted or unsubstituted C₁-C₂₄alkylen, preferably C₁-C₆alkylen, wherein one or more, preferably non-adjacent, —CH₂— group may be replaced by a linking group, preferably replaced by —O—, —O(CO)—, —(O)O—, —NR₁CO—, —CONR₁—, wherein R₁ is hydrogen or C₁-C₆alkyl; most preferably S⁰ is a single bond;
M represents a H, C₁-C₂₄alkyl and CF₃,
L is —CH₃, —COCH₃, —OCH₃, nitro, cyano, halogen, CH₂═CH—, CH₂═C(CH₃)—, CH₂═CH—(CO)O—, CH₂═CH—O—, —NR⁵R⁶, CH₂═C(CH₃)—(CO)O— or CH₂═C(CH₃)—O—,
   wherein:
R⁵, R⁶ each independently from each other represents a hydrogen atom or C₁-C₆alkyl and/or an alicyclic group;
u₃ is an integer from 0 to 2;
preferably D is selected from the group of the following compounds IIa, IIb and IIj, more preferably D is selected from the following compounds IIa and IIb;
E represents an aromatic group, an oxygen atom, a sulphur atom, —NH—, —N(C₁-C₆alkyl)-, —CR²R³,
   wherein R² and R³ are independently from each other hydrogen or a cyclic, straight-chain or branched, substituted or unsubstituted C₁-C₂₄alkyl, wherein one or more —CH₂— group(s) may be independently from each other replaced by a linking group, and with the proviso that at least one of R² and R³ is not hydrogen;
S¹ represents a single bond or a cyclic, straight-chain or branched, substituted or unsubstituted C₁-C₂₄alkylen, if D is a compound of formulae IIa, IIc, IId, IIe, IIf, IIg, IIh, IIi, IIj, IIk, preferably D is a compound of formulae IIa, IIj and IIk; and if D is a compound of formula IIb, S¹ represents a single bond or a cyclic, straight-chain or branched, substituted or unsubstituted C₂-C₂₄alkylen, preferably C₅-C₂₄alkylen, more preferably C₁₀-C₂₄alkylen; wherein one or more, preferably non-adjacent, —CH₂— groups of the C₁-C₂₄alkylen, C₂-C₂₄alkylen, C₅-C₂₄alkylen or C₁₀-C₂₄alkylen may independently from each other be replaced by a linking group as described above and/or a non-aromatic, aromatic, unsubstituted or substituted carbocyclic or heterocyclic group connected via bridging groups,
S² represents a spacer unit;
X, Y each independently from each other represents hydrogen, fluorine, chlorine, cyano, unsubstituted or with fluorine substituted C₁-C₁₂alkyl, in which one or more —CH₂— groups may be replaced by a linking group;
n, n1 each independently from each other represents 1, 2, 3 or 4, preferably n1 is 1 and n is 1 or 2;
   with the proviso that if n is 2, 3, or 4, each A, B, x₁, E, S¹, S², X, Y are identical or different; and if n1 is 2, 3 or 4 each B, x₁ is identical or different;
   preferably,
   wherein, if n>1, compound (I) has several side-chains [wherein side-chain has the meaning of structures (I) without the group D], which are linked to residue D at one atomic position within group D, e.g. two or three side chains linked to one single carbon atom within group D, or they can be linked to group D at different atomic positions within group D, e.g. at adjacent atomic positions within group D, or/and they can linked spaced further apart.

In a more preferred embodiment the present invention relates to diamine of formula (I):

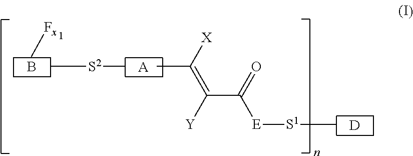

wherein,
A represents a unsubstituted or substituted carbocyclic or heterocyclic aromatic group selected from a monocyclic ring of five or six atoms, two adjacent monocyclic rings of five or six atoms, a bicyclic ring system of eight, nine or ten atoms, or a tricyclic ring system of thirteen or fourteen atoms;
F is fluorine, and
x₁ is an integer from 0 to 15,
B represents a straight-chain or branched C₁-C₁₆alkyl, which is unsubstituted or substituted by di-(C₁-C₁₆alkyl)amino, C₁-C₆alkyloxy, nitro, cyano and/or chlorine or fluorine; and wherein one or more —CH₂— group may independently be replaced by a linking group;
E represents an aromatic group, an oxygen atom, a sulphur atom, —NH—, —N(C₁-C₆alkyl)-, —CR²R³,
   wherein R² and R³ are independently from each other hydrogen or a cyclic, straight-chain or branched, substituted or unsubstituted C₁-C₂₄alkyl, wherein one or more —CH₂— groups may be replaced by a linking group, and with the proviso that at least one of R² and R³ is not hydrogen;
S¹, S² have the same meaning and preferences as described above;
D has the same meaning as described above, and signifies preferably a group of formulae IIa, IIc, IId, IIe, IIf, IIg, IIh, IIi, IIj, IIk, if S¹ represents a single bond or a cyclic, straight-chain or branched, substituted or unsubstituted C₁-C₂₄alkylen and if S¹ represents a single bond or a cyclic, straight-chain or branched, substituted or unsubstituted C₂-C₂₄alkylen D is IIb, preferably D is a group of formulae IIa, IIj and IIk if S¹ represents a single bond or a cyclic, straight-chain or branched, substituted or unsubstituted C₁-C₂₄alkylen;
X, Y each independently from each other represents hydrogen, fluorine, chlorine, cyano, unsubstituted or with fluorine substituted C₁-C₁₂alkyl, in which one or more —CH₂— groups may be replaced by a linking group;
n is 1, 2, 3 or 4,
   with the proviso that if n is 2, 3, or 4, each A, B, x₁, D, E, S¹, S², X, Y may be identical or different.

The term “linking group”, as used in the context of the present invention is preferably be selected

from —O—, —CO, —CO—O—, —O—CO—,

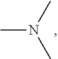

—NR¹—, —NR¹—CO—, —CO—NR¹—, —NR¹—CO—O—, —O—CO—NR¹—, —NR¹—CO—NR¹—, —CH═CH—, —C≡C—, —O—CO—O—, and —Si(CH₃)₂—O—Si(CH₃)₂—, and wherein:
R¹ represents a hydrogen atom or C₁-C₆alkyl;
   with the proviso that oxygen atoms of linking groups are not directly linked to each other.

The term “spacer unit” as used in the context of the present invention, is preferably a single bond, a cyclic, straight-chain or branched, substituted or unsubstituted C₁-C₂₄alkylen, wherein one or more, preferably non-adjacent, —CH₂— groups may independently from each other be replaced by a linking group as described above and/or a non-aromatic, aromatic, unsubstituted or substituted carbocyclic or heterocyclic group connected via bridging groups.

More preferably, the spacer unit is a cyclic, straight-chain or branched, substituted or unsubstituted C₁-C₂₄alkylen, wherein one or more, preferably non-adjacent, —CH₂— groups may independently from each other be replaced by a linking group and/or a non-aromatic, aromatic, unsubstituted or substituted carbocyclic or heterocyclic group connected via bridging groups.

A bridging group as used in the context of the present invention is preferably selected from —CH(OH)—, —CO—, —CH₂(CO)—, —SO—, —CH₂(SO)—, —SO₂—, —CH₂(SO₂)—, —COO—, —OCO—, —COCF₂—, —CF₂CO, —S—CO—, —CO—S—, —SOO—, —OSO—O, —SOS—, —O—CO—O—, —CH₂—CH₂—, —OCH₂—, —CH₂O—, —CH═CH—, —C≡C—, —CH═CH—COO—, —OCO—CH═CH—, —CH═N—, —C(CH₃)═N—, —N═N— or a single bond; or a cyclic, straight-chain or branched, substituted or unsubstituted C₁-C₂₄alkylen, wherein one or more —CH₂— groups may independently from each other be replaced by a linking group as described above.

Alkyl, alkyloxy, alkylcarbonyloxy, acryloyloxyalkoxy, acryloyloxyalkyl, acryloyloxyalken, alkyloxycarbonyloxy, alkylacryloyloxy, methacryloyloxyalkoxy, methacryloyloxyalkyl, methacryloyloxyalken, alkylmethacryloyloxy, alkylmethacryloyloxy, alkylvinyl, alkylvinyloxy and alkylallyloxy and alkylene, as used in the context of the present invention denote with their alkyl residue, respectively their alkylene residue, a cyclic, straight-chain or branched, substituted or unsubstituted alkyl, respectively alkylene, in which one or more, preferably non-adjacent, —CH₂— group may be replaced by a linking group.

Further, the alkyl residue is for example C₁-C₄₀alkyl, especially C₁-C₃₀alkyl, preferably C₁-C₂₀alkyl, more preferably C₁-C₁₆alkyl, most preferably C₁-C₁₀alkyl and especially most preferably C₁-C₆alkyl. Accordingly alkylen is for example C₁-C₄₀alkylen, especially C₁-C₃₀alkylen, preferably C₁-C₂₀alkylen, more preferably C₁-C₁₆alkylen, most preferably C₁-C₁₀alkylen and especially most preferably C₁-C₆alkylen.

In the context of the present invention the definitions for alkyl given below, are applicable to alkylene in analogy.

C₁-C₆alkyl is for example methyl, ethyl, propyl, isopropyl, butyl, sec.-butyl, tert.-butyl, pentyl or hexyl.

C₁-C₁₀alkyl is for example methyl, ethyl, propyl, isopropyl, butyl, sec.-butyl, tert.-butyl, pentyl, hexyl, heptyl, octyl, nonyl, decyl.

C₁-C₁₆alkyl is for example methyl, ethyl, propyl, isopropyl, butyl, sec.-butyl, tert.-butyl, pentyl, hexyl, heptyl, octyl, nonyl, decyl, undecyl, dodecyl, tridecyl, tetradecyl, pentadecyl or hexadecyl.

C₁-C₂₀alkyl is for example methyl, ethyl, propyl, isopropyl, butyl, sec.-butyl, tert.-butyl, pentyl, hexyl, heptyl, octyl, nonyl, decyl, undecyl, dodecyl, tridecyl, tetradecyl, pentadecyl, hexadecyl, heptadecyl, octadecyl, nondecyl, eicosyl.

C₁-C₂₄alkyl is for example methyl, ethyl, propyl, isopropyl, butyl, sec.-butyl, tert.-butyl, pentyl, hexyl, heptyl, octyl, nonyl, decyl, undecyl, dodecyl, tridecyl, tetradecyl, pentadecyl, hexadecyl, heptadecyl, octadecyl, nondecyl, eicosyl.

C₁-C₃₀alkyl is for example methyl, ethyl, propyl, isopropyl, butyl, sec.-butyl, tert.-butyl, pentyl, hexyl, heptyl, octyl, nonyl, decyl, undecyl, dodecyl, tridecyl, tetradecyl, pentadecyl, hexadecyl, heptadecyl, octadecyl, nondecyl, eicosyl, heneicosyl, tricosyl, tetracosy, pentacosyl, hexacosdy, heptacosyl, octacosyl, nonacosy or triacontyl.

C₁-C₄₀alkyl is for example methyl, ethyl, propyl, isopropyl, butyl, sec.-butyl, tert.-butyl, pentyl, hexyl, heptyl, octyl, nonyl, decyl, undecyl, dodecyl, tridecyl, tetradecyl, pentadecyl, hexadecyl, heptadecyl, octadecyl, nondecyl, eicosyl, heneicosyl, tricosyl, tetracosy, pentacosyl, hexacosdy, heptacosyl, octacosyl, nonacosy, triacontyl or tetracontyl.

C₁-C₂₀acryloyloxyalkylene, preferably C₁-C₁₀acryloyloxyalkylene, C₁-C₆ acryloyloxyalkylene is for example acryloyloxymethylen, acryloyloxyethylene, acryloyloxypropylene, acryloyloxyisopropylene, acryloyloxybutylene, acryloyloxy-sec.-butylene, acryloyloxypentylene, acryloyloxyhexylene, acryloyloxyheptylene, acryloyloxyoctylene, acryloyloxynonylene, acryloyloxydecylene, acryloyloxyundecylene, acryloyloxydodecane, acryloyloxytridecylene, acryloyloxytetradecylene, acryloyloxypentyldecane, acryloyloxyhexadecylene, acryloyloxyheptadecylene, acryloyloxyoctadecylene, acryloyloxynondecylene, acryloyloxyeicosylene.

C₁-C₂₀methacryloyloxyalkylene, preferably C₁-C₁₀methacryloyloxyalkylene, C₁-C₆ methacryloyloxyalkylene is for example methacryloyloxymethylen, methacryloyloxyethylene, methacryloyloxypropylene, methacryloyloxyisopropylene, methacryloyloxybutylene, methacryloyloxy-sec.-butylene, methacryloyloxypentylene, methacryloyloxyhexylene, methacryloyloxyheptylene, methacryloyloxyoctylene, methacryloyloxynonylene, methacryloyloxydecylene, methacryloyloxyundecylene, methacryloyloxydodecane, methacryloyloxytridecylene, methacryloyloxytetradecylene, methacryloyloxypentyldecane, methacryloyloxyhexadecylene, methacryloyloxyheptadecylene, methacryloyloxyoctadecylene, methacryloyloxynondecylene, methacryloyloxyeicosylene.

C₁-C₂₀acryloyloxyalkoxy, preferably C₁-C₁₀acryloyloxyalkoxy, C₁-C₆ acryloyloxyalkoxy is for example acryloyloxymethoxy, acryloyloxyethoxy, acryloyloxypropoxy, acryloyloxyisopropoxy, acryloyloxybutoxy, acryloyloxy-sec.-butoxy, acryloyloxypentoxy, acryloyloxyhexoxy, acryloyloxyheptoxy, acryloyloxyoctoxy, acryloyloxynonoxy, acryloyloxydecoxy, acryloyloxyundecoxy, acryloyloxydodecanoxy, acryloyloxytridecyloxy.

C₁-C₂₀methacryloyloxyalkoxy, preferably C₁-C₁₀methacryloyloxyalkoxy, C₁-C₆ methacryloyloxyalkoxy is for example methacryloyloxymethoxy, methacryloyloxyethoxy, methacryloyloxypropoxy, methacryloyloxyisopropoxy, methacryloyloxybutoxy, methacryloyloxy-sec.-butoxy, methacryloyloxypentoxy, methacryloyloxyhexoxy, methacryloyloxyheptoxy, methacryloyloxyoctoxy, methacryloyloxynonoxy, methacryloyloxydecoxy, methacryloyloxyundecoxy, methacryloyloxydodecanoxy, methacryloyloxytridecyloxy.

An aliphatic group is for example a saturated or unsaturated, mono-, bi-, tri-, tetra-, penta-, hexa-, hepta-, octa-, nona-, deca-valent alkyl, alkylene, alkyloxy, alkylcarbonyloxy, acryloyloxy, alkylacryl, alkylmethacryl, alkyl(en)acryl(en), alkyl(en)methacryl(en), alkyloxycarbonyloxy, alkyloxycarbonyloxy methacryloyloxy, alkylvinyl, alkylvinyloxy or alkylallyloxy, which may comprise one or more heteroatom and/or bridging group.

An alicyclic group is preferably a non-aromatic group or unit. Preferably an alicyclic group is a non-aromatic carbocyclic or heterocyclic group and represents for example ring systems, with 3 to 30 carbon atoms, as for example cyclopropane, cyclobutane, cyclopentane, cyclopentene, cyclohexane, cyclohexene, cyclohexadiene, decaline, tetrahydrofuran, dioxane, pyrrolidine, piperidine or a steroidal skeleton such as cholesterol.

The term “aromatic”, as used in the context of the present invention, preferably denotes unsubstituted or substituted carbocyclic and heterocyclic groups, incorporating five, six, ten of 14 ring atoms, e.g. furan, benzene or phenylene, pyridine, pyrimidine, naphthalenen, which may form ring assemblies, such as biphenylene or triphenylen, which are uninterrupted or interrupted by at least a single heteroatom and/or at least a single bridging group; or fused polycyclic systems, such as phenanthrene, tetraline. Preferably aromatic group are benzene, phenylene, biphenylene or triphenylen. More preferred aromatic group are benzene, phenylene and biphenylene.

A carbocyclic or heterocyclic aromatic group incorporates preferably five, six, ten or 14 ring atoms, as for example furan, benzene, pyridine, triazine, pyrimidine, naphthalene, phenanthrene, biphenylene or tetraline units, preferably naphthalene, phenanthrene, biphenylene or phenylene, more preferably naphthalene, biphenylene or phenylene, and most preferably phenylene.

The carbocyclic or heterocyclic aromatic group is for example unsubstituted or mono- or poly-substituted. Preferred substitutents of carbocyclic or heterocyclic aromatic groups are at least one halogen, hydroxyl, a polar group, acryloyloxy, alkylacryloyloxy, alkoxy, alkylcarbonyloxy, alkyloxycarbonyloxy, alkyloxocarbonyloxy, methacryloyloxy, vinyl, vinyloxy and/or allyloxy group, wherein the alkyl residue has preferably from 1 to 20 carbon atoms, and more preferably having from 1 to 10 carbon atoms. Preferred polar groups are nitro, cyano or a carboxy group, and/or a cyclic, straight-chain or branched C₁-C₃₀alkyl, which is unsubstituted, mono- or poly-substituted. Preferred substitutents of C₁-C₃₀alkyl are methyl, fluorine and/or chlorine, wherein one or more, preferably non-adjacent, —CH₂— group may independently of each other be replaced by a linking group. Preferably, the linking group is selected from —O—, —CO—, —COO— and/or —OCO—.

A monocyclic ring of five or six atoms is for example furan, benzene, preferably phenylene, pyridine, pyrimidine.

A bicyclic ring system of eight, nine or ten atoms is for example naphthalene, biphenylene or tetraline.

A tricyclic ring system of thirteen or fourteen atoms is for example phenanthrene. The term “phenylene”, as used in the context of the present invention, preferably denotes a 1,2-, 1,3- or 1,4-phenylene group, which is optionally substituted. It is preferred that the phenylene group is either a 1,3- or a 1,4-phenylene group. 1,4-phenylene groups are especially preferred.

The term “halogen” denotes a chloro, fluoro, bromo or iodo substituent, preferably a chloro or fluoro substituent.

The term “polar group”, as used in the context of the present invention primarily denotes a group like a nitro, cyano, or a carboxy group.

The term “heteroatom”, as used in the context of the present invention primarily denotes oxygen, sulphur and nitrogen, preferably oxygen and nitrogen, in the latter case preferably in the form of —NH—.

The term “optionally substituted” as used in the context of the present invention primarily means substituted by lower alkyl, such as C₁-C₆alkyl, lower alkoxy, such as C₁-C₆alkoxy, hydroxy, halogen or by a polar group as defined above.

The term “diamine” or “diamine compound” is to be understood as designating a chemical structure which has at least two amino groups, i.e. which may also have 3 or more amino groups. The at least two amino groups are preferably able to react with e.g. anhydrides as outlined in more detail below.

The term “dinitro” or “dinitro compound” is to be understood as designating a chemical structure which has at least two nitro groups, i.e. which may also have 3 or more nitro groups, and wherein the dinitro group is a precursor compound of the “diamino compound”. The dinitro compound is conventionally converted to the diamino compound by reduction methods known in the art.

With respect to straight chain or branched alkyl, alkylene, alkoxy, alkylcarbonyloxy, acryloyloxyalkoxy, acryloyloxyalkyl, acryloyloxyalkene, alkyloxycarbonyloxy, alkylacryloyloxy, methacryloyloxyalkoxy, methacryloyloxyalkyl, methacryloyloxyalkene, alkylmethacryloyloxy, alkylmethacryloyloxy, alkylvinyl, alkylvinyloxy, alkylallyloxy and alkylene groups it is repeatedly pointed out that some or several of the —CH₂— groups may be replaced e.g. by heteroatoms, but also by other groups, preferably bridging groups. In such cases it is generally preferred that such replacement groups are not directly linked to each other. It is alternatively preferred that heteroatoms, and in particular oxygen atoms are not directly linked to each other.

Preferably, A is unsubstituted or substituted phenanthrylene, naphthylene, biphenylene or phenylene, wherein the preferred subsituent(s) is (are) a halogen atom, a hydroxy group and/or by a polar group, wherein the polar group is preferably nitro, cyano, carboxy; and/or by acryloyloxy, alkylacryl, alkylmethacryl, alkyl(en)acryl, alkyl(en)methacryl, acrylenacryl, methacrylenalkyl, methacryloyloxy, vinyl, vinyloxy, allyl, allyloxy, and/or by a cyclic, straight-chain or branched alkyl, which is unsubstituted, mono- or poly-substituted by fluorine and/or chlorine, having from 1 to 20 carbon atoms, wherein one or more, preferably non-adjacent, —CH₂— groups may independently be replaced by a linking group and or an aromatic or an alicyclic group, preferably the linking group is selected from —O—, —CO—, —CO—O—, —O—CO—.

More preferably A is substituted or unsubstituted naphthylene, biphenylene or phenylene, wherein the preferred subsituent(s) is (are) halogen atom, hydroxy group and/or by acryloyloxy, alkylacryl, alkylmethacryl, acrylenacryl, methacrylenalkyl, methacryloyloxy, straight-chain or branched alkyl, alkoxy, alkylcarbonyloxy, and/or alkyloxycarbonyl groups, wherein the alkyl residue has from 1 to 20 carbon atoms.

Most preferably A is substituted or unsubstituted phenylene, preferably 1,4-phenylen, wherein the preferred subsituent(s) is (are) a halogen atom, and/or by acryloyloxy or methacryloyloxy, and/or by an alkoxy, alkylacryl, alkylmethacryl, acrylenacryl, methacrylenalkyl, alkylcarbonyloxy, and/or alkyloxycarbonyl groups, wherein the alkyl residue has from 1 to 10 carbon atoms.

A preferred embodiment of the present invention concerns a diamine compound of formula (I) as described above, wherein the following compound residue (Ia)

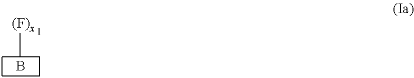

represents a straight-chain or branched C₁-C₁₆alkyl or group C₁-C₁₆fluoralkyl group with terminal units selected from —CF₂H and —CF₃, preferably selected from —CF₂H or —CF₃, —CF₂CF₃, —CF₂CHF₂, —(CF₂)₂CF₃, —(CF₂)₂CHF₂, —(CF₂)₃CHF₂, —(CF₂)₃CF₃, —CF(CF₃)₂ and —CF₂(CHF)CF₃.

Preferably B is a straight-chain or branched C₁-C₁₂alkyl, wherein one or more, preferably non-adjacent, —CH₂— group(s) may independently from each other be replaced by a group selected from

—O—, —CO, —CO—O—, —O—CO—, —NR¹—, —NR¹—CO—, —CO—NR¹—, —NR¹—CO—O—, —O—CO—NR¹—, —NR¹—CO—NR¹—, —CH═CH—, —C≡C—, —O—CO—O—, and —Si(CH₃)₂—O—Si(CH₃)₂—, an aromatic and an alicyclic group; and wherein:
R¹ represents a hydrogen atom or C₁-C₆alkyl;
   with the proviso that oxygen atoms are not directly linked to each other.

More preferably, B is a straight-chain or branched C₁-C₁₂alkyl, wherein one or more, preferably non-adjacent, —CH₂— group(s) may be replaced by a group selected from from —O—, —CO, —CO—O—, —O—CO—, —NR¹, —NR¹—CO—, —CO—NR¹— or —CH═CH— wherein:
R¹ represents a hydrogen atom or C₁-C₆alkyl;
   with the proviso that oxygen atoms are not directly linked to each other.

Most preferably, B is a straight-chain or branched C₁-C₈alkyl, wherein one or more, preferably non-adjacent, —CH₂— group(s) may be replaced by a group selected from from —O—, —CO, —CO—O—, —O—CO—, —NR¹, —NR¹—CO—, —CO—NR¹— or —CH═CH— wherein:
R¹ represents a hydrogen atom or C₁-C₆alkyl;
   with the proviso that oxygen atoms are not directly linked to each other.

Especially most preferably, B is a straight-chain or branched C₁-C₈alkyl, wherein one or more, preferably non-adjacent, the —CH₂— group may be replaced by a group selected from —O—, —CO—, —CO—O—, —O—CO—, and —CH═CH—, with the proviso that oxygen atoms are not directly linked to each other.

Preferably the compound residue (Ia) is:

trifluoromethyl; 2,2,2-trifluoroethyl; difluoromethyl; pentafluoroethyl; 2,2-tetrafluoroethyl; 3,2-tetrafluoroethyl; 3,3,3-trifluoropropyl; 2,2,3,3-tetrafluoropropyl; 2,2,3,3,3-pentafluoropropyl; hexafluoropropyl; heptafluoropropyl; 4,4,4-trifluorobutyl; tetrafluorobutyl; 3,3,4,4,4-pentafluorobutyl; hexafluorobutyl; 2,2,3,3,4,4,4-heptafluorobutyl; 5,5,5-trifluoropentyl; tetrafluoropentyl; 4,4,5,5,5-pentafluoropentyl; hexafluoropentyl; 3,3,4,4,5,5,5-heptafluoropentyl; 6,6,6-trifluorohexyl; tetrafluorohexyl; 5,5,6,6,6-pentafluorohexyl; hexafluorohexyl; 4,4,5,5,6,6,6-heptafluorohexyl; nonafluorohexyl;
1-trifluoro-1,2,2,2-tertafluoroethoxy, 2-trifluoro-2,3,3,3-tertafluoropropoxy, 3-trifluoro-3,4,4,4-tertafluorobutoxy, 4-trifluoro-4,5,5,5-tertafluoropentoxy, 5-trifluoro-5,6,6,6-tertafluorohexoxy, 6-trifluoro-6,7,7,7-tertafluoroheptoxy, 7-trifluoro-7,8,8,8-tertafluorononoxy;
fluoroalkoxy derivatives, such as
trifluoromethoxy; 2,2,2-trifluoroethoxy; difluoromethoxy; pentafluoroethoxy; 1,1,2,2-tetrafluoroethoxy; 2,2,2,1-tetrafluoroethoxy; 3,3,3-trifluoropropoxy; 2,2,3,3-tetrafluoropropoxy; 2,2,3,3,3-pentafluoropropoxy; hexafluoropropoxyl; heptafluoropropoxy; 4,4,4-trifluorobutoxy; tetrafluorobutoxy; 3,3,4,4,4-pentafluorobutoxy; 2,2,3,3,4,4-hexafluorobutoxy; 2,2,3,3,4,4,4-heptafluorobutoxy; 5,5,5-trifluoropentoxy; tetrafluoropentoxy; 4,4,5,5,5-pentafluoropentoxy; hexafluoropentoxy; 3,3,4,4,5,5,5-heptafluoropentoxy; 6,6,6-trifluorohexoxy; tetrafluorohexoxy; 5,5,6,6,6-pentafluorohexoxy; hexafluorohexoxy; 4,4,5,5,6,6,6-heptafluorohexoxy; nonafluorohexoxy;
trifluoromethylen carbamate; 2,2,2-trifluoroethylen carbamate; difluoromethylen carbamate; pentafluoroethylen carbamate; 2,2-tetrafluorethylen carbamate; 3,2-tetrafluorethylen carbamate; 3,3,3-trifluoropropylen carbamate; 2,2,3,3-tetrafluoropropylen carbamate; 2,2,3,3,3-pentafluoropropylen carbamate; hexafluoropropylen carbamate; heptafluoropropylen carbamate; 4,4,4-trifluorobutylen carbamate; tetrafluorobutylen carbamate; 3,3,4,4,4-pentafluorobutylen carbamate; hexafluorobutylen carbamate; 2,2,3,3,4,4,4-heptafluorobutylen carbamate; 5,5,5-trifluoropentylen carbamate; tetrafluoropentylen carbamate; 4,4,5,5,5-pentafluoropentylen carbamate; hexafluoropentylen carbamate; 3,3,4,4,5,5,5-heptafluoropentylen carbamate; 6,6,6-trifluorohexylen carbamate; tetrafluorohexylen carbamate; 5,5,6,6,6-pentafluorohexylen carbamate; hexafluorohexylen carbamate; 4,4,5,5,6,6,6-heptafluorohexylen carbamate; nonafluorohexylen carbamate;
fluoroalkyloyloxy derivatives, such as
trifluoromethyloyloxy; 2,2,2-trifluoroethyloyloxy; pentafluoroethyloyloxy; 1,1,2,2-tetrafluorethyloyloxy; 2,2,2,1-tetrafluorethyloyloxy; 3,3,3-trifluoropropyloyloxy; tetrafluoropropyloyloxy; 2,2,3,3,3-pentafluoropropyloyloxy; hexafluoropropyloyloxy; 1,1,2,2,3,3,3-heptafluoropropyloyloxy; 4,4,4-trifluorobutyloyloxy; tetrafluorobutyloyloxy; 3,3,4,4,4-pentafluorobutyloyloxy; hexafluorobutyloyloxy; 2,2,3,3,4,4,4-heptafluorobutyloyloxy; 5,5,5-trifluoropentyloyloxy; tetrafluoropentyloyloxy; 4,4,5,5,5-pentafluoropentyloyloxy; hexafluoropentyloyloxy; 3,3,4,4,5,5,5-heptafluoropentyloyloxy; 6,6,6-trifluorohexyloyloxy; tetrafluorohexyloyloxy; 5,5,6,6,6-pentafluorohexyloyloxy; hexafluorohexyloyloxy; 4,4,5,5,6,6,6-heptafluorohexyloyloxy; trifluoroacetyl; nonafluorohexyloyloxy;
4,4,4-trifluorobut-2-enyl; 5,5,5-trifluoropent-1-enyl; 6,6,6-trifluorohex-1-enyl; 7,7,7-trifluorohept-1-enyl; trifluoroacetylaminomethoxy; trifluoroacetylaminoethoxy; trifluoroacetylaminopropoxy; trifluoroacetylaminobutoxy; 2-fluoroethyl; 3-fluoropropyl; 4-fluorobutyl; 5-fluoropentyl; 6-fluorohexyl; 2-fluoroethoxy; 3-fluoropropoxy; 4-fluorobutoxy; 5-fluoropentoxy; 6-fluorohexyloxy; 4-fluorobut-1-enyl; 5-fluoropent-1-enyl; 6-fluorohex-1-enyl; 7-fluorohept-1-enyl; 4,4,4-trifluoro-3-(trifluoromethyl)butoxy; 4,5,5-trifluoropent-4-enoxy; 4,5,5-trifluoropent-4-enoyloxy; 5,6,6-trifluorohex-5-enoxy or 5,6,6-trifluoropent-5-enoyloxy;
or
methyl; ethyl; propyl; propyl; butyl; pentyl; hexyl; heptyl, octyl, nonyl, decyl, undecyl, dodecyl, tridecyl, tetradecyl, pentadecyl, hexadecyl, heptadecyl, octadecyl, nondecyl, eicosyl;
methoxy; ethoxy, propoxy, butoxy, pentoxy, hexoxy, heptoxy, octoxy, nonoxy, decoxy, undecoxy, dodecoxy, tridecoxy, teteradecoxy, pentadecoxy, hexadecoxy, heptadecoxy, octadecoxy, nondecoxy, eicosoxy;
methylen carbamate; ethylen carbamate; propylen carbamate; butylen carbamate; pentylen carbamate; hexylen carbamate; heptylen carbamate, octylen carbamate, nonylen carbamate, decylen carbamate, undecylen carbamate, dodecylen carbamateyl, tridecylen carbamate, tetradecylen carbamateyl, pentadecylen carbamate, hexadecylen carbamate, heptadecylen carbamate, octadecylen carbamateyl, nondecylen carbamate, eicosylen carbamate;
methyloyloxy; ethyloyloxy; propyloyloxy; butyloyloxy; pentyloyloxy; hexyloyloxy; heptyloyloxy, octyloyloxy, nonyloyloxy, decyloyloxy, undecyoyloxyl, dodecyloyloxy, tridecyloyloxy, tetradecyloyloxy, pentadecyloyloxy, hexadecyloyloxy, heptadecyloyloxy, octadecyloyloxy, nondecyloyloxy, eicosyloyloxy;
ethenyl, propenyl, but-2-enyl; pent-1-enyl; hex-1-enyl; hept-1-enyl; acetylaminomethoxy; acetylaminoethoxy; acetylaminopropoxy; acetylaminobutoxy;

The diamine groups D are commercial available or accessible by known methods. The second amino group is accessible for example by substitution reaction.

E preferably represents an phenylene, an oxygen atom or a —N(H)— group, more preferred E is oxygen or a —N(H)— group, and most preferred E is oxygen.
Preferably, S¹ represents a single bond or a cyclic, straight-chain or branched, substituted or unsubstituted C₁-C₂₄alkylen, if D is a compound of formulae IIa, IIc, IId, IIe, IIf, IIg, IIh, IIi, IIj, IIk, preferably D is a compound of formulae IIa, IIj and IIj; and if D is a compound of formula IIb, S¹ represents a single bond or a cyclic, straight-chain or branched, substituted or unsubstituted C₂-C₂₄alkylen, preferably C₅-C₂₄alkylen, more preferably C₁₀-C₂₄alkylen;
S² represents a single bond or a C₁-C₂₄alkylen, which is a cyclic, straight-chain or branched, substituted or unsubstituted C₁-C₂₄alkylen,
   and
wherein in S¹ or S² independently from each other one or more, preferably non-adjacent, —CH₂— groups may independently from each other be replaced by a linking group as described above and/or a non-aromatic, aromatic, unsubstituted or substituted carbocyclic or heterocyclic group of formula (IV):

—(Z¹—C¹)ₐ₁—(Z²—C²)ₐ₂—(Z^{1a})ₐ₃— (IV)

wherein:
C¹, C² each independently represents a non-aromatic, aromatic, optionally substituted carbocyclic or heterocyclic group, preferably connected to each other via the bridging groups Z¹ and Z² and/or Z^{1a}, preferably C¹ and C² are connected at the opposite positions via the bridging groups Z¹ and Z² and/or Z^{1a}, so that groups S¹ and/or S² have a long molecular axis, and
Z¹, Z², Z^{1a} each independently represents a bridging group, preferably selected from —CH(OH)—, —CH₂—, —O—, —CO—, —CH₂(CO)—, —SO—, —CH₂(SO)—, —SO₂—, —CH₂(SO₂)—, —COO—, —OCO—, —COCF₂—, —CF₂CO—, —S—CO—, —CO—S—, —SOO—, —OSO—, —SOS—, —CH₂—CH₂—, —OCH₂—, —CH₂O—, —CH═CH—, —C≡C—, —CH═CH—COO—, —OCO—CH═CH—, —CH═N—, —C(CH₃)═N—, —O—CO—O—, —N═N— or a single bond; and
a₁, a₂, a₃ each independently represents an integer from 0 to 3, such that a₁+a₂+a₃≦6, wherein preferably S² is linked to A via Z¹; preferably a₃ is 0 and a₁+a₂≦4.

More preferred S¹ represents a single bond or a cyclic, straight-chain or branched, substituted or unsubstituted C₁-C₂₄alkylen, if D is a compound of formulae IIa, IIc, IId, IIe, IIf, IIg, IIh, IIi, IIj, IIk, preferably D is a compound of formulae IIa, IIj and IIk; and if D is a compound of formula IIb, S¹ represents a single bond or a cyclic, straight-chain or branched, substituted or unsubstituted C₂-C₂₄alkylen, preferably C₅-C₂₄alkylen, more preferably C₁₀-C₂₄alkylen; wherein one or more —CH₂— group may independently be replaced by a linking group or/and a group represented by the formula (IV), wherein:
C¹, C² are selected from a compound of group G¹, wherein group G¹ is:

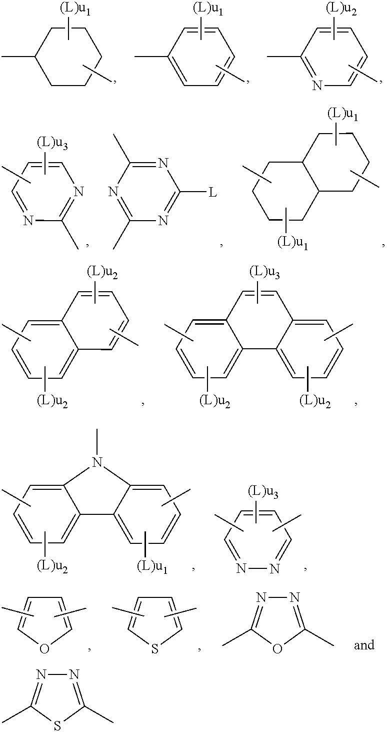

wherein:
“-” denotes the connecting bonds of C¹ and C² to the adjacent groups in formula (IV); and
L is —CH₃, —OCH₃, —COCH₃, nitro, cyano, halogen, CH₂═CH—, CH₂═C(CH₃)—, CH₂═CH—(CO)O—, CH₂═CH—O—, CH₂═C(CH₃)—(CO)O—, or CH₂═C(CH₃)—O—,
u₁ is an integer from 0 to 4; and
u₂ is an integer from 0 to 3; and
u₃ is an integer from 0 to 2; and
Z¹, Z², Z^{1a} each independently represents —O—, —CO—, —COO—, —OCO—, —COCF₂—, —CF₂CO—, —CH₂—CH₂—, —OCH₂—, —CH₂O—, —CH═CH—, —C≡C—, —CH═CH—COO—, —OCO—CH═CH— or a single bond; with the proviso that heteroatoms are not directly linked to each other, and
a₁, a₂, a₃ each independently represents an integer from 0 to 3, such that a₁+a₂+a₃≦6; preferably a₃ is 0 and a¹+a²≦4.
Most preferred S¹ represents a single bond or a spacer unit such as a straight-chain or branched C₁-C₁₄alkylen D is a compound of formulae IIa, IIc, IId, IIe, IIf, IIg, IIh, IIi, IIj, IIk, preferably D is a compound of formulae IIa, IIj and IIk; and if D is a compound of formula IIb, S¹ represents a single bond or a cyclic, straight-chain or branched, substituted or unsubstituted C₂-C₁₄alkylen, preferably C₅-C₁₄alkylen, more preferably C₁₀-C₁₄alkylen;
   wherein one or more, preferably non adjactent, —CH₂— group may independently be replaced by a linking group and/or a group represented by formula (IV), wherein:
C¹, C² each independently represents a 1,4-phenylene, 2-methoxy-1,4-phenylene, 1,4-cyclohexylene or a 4,4′-biphenylene group; and
Z¹, Z², Z^{1a} each independently represents —COO—, —OCO—, —CH₂—CH₂—, —OCH₂—, —CH₂O—, —CH═CH—, —C≡C—, —CH═CH—COO—, —OCO—CH═CH— or a single bond; and
a₁, a₂, a₃ are independently 0 or 1, preferably a₃ is 0.

Especially most preferred S¹ represents a single bond or a cyclic, straight-chain or branched, substituted or unsubstituted C₁-C₁₄alkylen, if D is a compound of formulae IIa, IIc, lid, IIe, IIf, IIg, IIh, IIi, IIj, IIk, preferably D is a compound of formulae IIa, IIj and IIk; and if D is a compound of formula IIb, S¹ represents a single bond or a cyclic, straight-chain or branched, substituted or unsubstituted C₂-C₁₄alkylen, preferably C₅-C₁₄alkylen, more preferably C₁₀-C₁₄alkylen, wherein one or more —CH₂— groups may be replaced by —O—, —O(CO)—, —(O)O—, —NR₁CO—, —CONR₁—, wherein R₁ is hydrogen or C₁-C₆alkyl or a group of formula (IV), wherein:
C¹, C² each independently represents 1,4-phenylene; and
Z¹, Z², Z^{1a} each independently represents —COO—, —OCO—, —CH₂—CH₂, —OCH₂—, —CH₂O—, —CH═CH—, —C≡C—, —CH═CH—COO—, —OCO—CH═CH—, or a single bond; and
a₁, a₂, a₃ are independently 0 or 1, preferably a₃ is 0.

More preferred S² represents a spacer unit such as a straight-chain or branched C₁-C₂₄alkylen, wherein one or more —CH₂— groups is independently replaced by a group represented by the formula (IV), wherein:
C¹, C² are selected from group G¹, with the avove given meaning; and
Z¹, Z², Z^{1a} each independently represents —O—, —CO—, —COO—, —OCO—, —COCF₂—, —CF₂CO—, —CH₂—CH₂—, —OCH₂—, —CH₂O—, —CH═CH—, —C≡C—, —CH═CH—COO—, —OCO—CH═CH— or a single bond; with the proviso that heteroatoms are not directly linked to each other, and
a₁, a₂, a₃ are each independently represents an integer from 0 to 3, such that a₁+a₂+a₃≦6, and preferably a₁+a₂≦4 and a₃ is 0; and wherein preferably S² is linked to A via Z¹.

Most preferred S² represents a straight-chain or branched C₁-C₁₂alkylen, wherein one or more —CH₂— group is independently be replaced by a group represented by the formula (IV), and more most preferred S² represents a group of formula (IV), wherein
C¹, C² each independently represents a 1,4-phenylene which is unsubstituted or mono or poly-substituted by a halogen atom, and/or by an alkoxy, alkylcarbonyloxy or an alkyloxycarbonyl group, having form 1 to 10 carbon atoms, 1,4-cyclohexylene or a 4,4′-biphenylene group; and
Z¹, Z², Z^{1a} each independently represents —COO—, —OCO—, —CH₂—CH₂—, —CH₂O—, —CH═CH—, —C≡C—, —CH═CH—COO—, —OCO—CH═CH— or a single bond; and
a₁, a₂, a₃ are independently 0 or 1, wherein preferably S² is linked to A via Z¹.

Especially most preferred S² represents a group of formula (IVa)

—(Z¹—C¹)ₐ_{₁}—(Z^{1a})ₐ_{₃}— (IVa)

wherein:
C¹ represents a non-aromatic, aromatic, unsubstituted or substituted carbocyclic or heterocyclic group, preferably selected from a compound of group G1, and
Z¹, Z^{1a} each independently from each other represent —COO—, —OCO—, —OCO(C₁-C₆)alkyl, —COOCH₂(C₁-C₈)alkyl-, —CH₂—CH₂—, —OCH₂—, —CH₂O—, —CH═CH—, —C≡C—, —CH═CH—COO—, —OCO—CH═CH—, or a single bond, or a straight-chain or branched, substituted or unsubstituted C₁-C₈alkylen, wherein one or more —CH₂— group may independently from each other be replaced by a linking group, preferably by —O—, as described above;
a₁, a₃ represents independently from each other 1, wherein preferably S² is linked to A via Z¹.

Further, especially most preferred S² represents a group of formula (IVa)

—(Z¹—C¹)ₐ_{₁}—(Z^{1a})ₐ_{₃}— (IVa)

wherein:
C¹ represents a 1,4-phenylene which is unsubstituted or mono or poly-substituted by a halogen atom, and/or by an alkoxy, alkylcarbonyloxy or an alkyloxycarbonyl group, having form 1 to 10 carbon atoms,
Z¹, Z^{1a} each independently from each other represent —COO—, —OCO—, —CH₂—CH₂—, —OCH₂—, —CH₂O—, —CH═CH—, —C≡C—, —CH═CH—COO—, —OCO—CH═CH—, or a single bond, or a straight-chain or branched, substituted or unsubstituted C₁-C₈alkylen, wherein one or more —CH₂— group may independently from each other be replaced by a linking group as described above, preferably by —O—, —COO—, —OCO—,
a₁, a₃ represents independently from each other 1, wherein preferably S² is linked to A via Z¹.
Another preferred embodiment of the present invention relates to a diamine compound (I), referring to any of the preceding definitions comprising these diamine compounds, wherein A represents phenanthrylene, biphenylene, naphthylene, or phenylene, which is unsubstituted or mono- or poly-substituted by a halogen atom, hydroxy group and/or by a polar group, preferably nitro, cyano, carboxy; and/or by acryloyloxy, methacryloyloxy, vinyl, vinyloxy, allyl, allyloxy, and/or by a cyclic, straight-chain or branched C₁-C₁₂alkyl residue, which is unsubstituted, mono- or poly-substituted by fluorine and/or chlorine, wherein one or more —CH₂— group may independently be replaced by a linking group and or an aromatic or an alicyclic group,
   and
   wherein the compound residue (Ia) of compound of formula (I) as described above

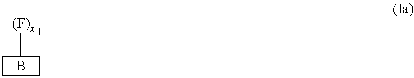

represents a straight-chain or branched C₁-C₁₂fluoralkyl group, wherein
F is fluorine, and
x₁ is an integer from 0 to 10,
B represents a straight-chain or branched C₁-C₁₂alkyl group, which is unsubstituted or in addition to its fluorine substituent(s) substituted by di-(C₁-C₁₆alkyl)amino, C₁-C₆alkyloxy, nitro, cyano and/or chlorine; and wherein one or more —CH₂— group may independently from each other be replaced by a linking group selected from —O—, —CO—, —CO—O—, —O—CO—, —NR¹—, —NR¹—CO—, —CO—NR¹— and —CH═CH—, wherein:
   R¹ represents a hydrogen atom or C₁-C₆alkyl;
   with the proviso that oxygen atoms are not directly linked to each other;
   and
   wherein the C₁-C₁₂fluoralkyl group has terminal units selected from —CF₂H and —CF₃, preferably selected from
   —CF₂H or —CF₃, —CF₂CF₃, —CF₂CHF₂, —(CF₂)₂CF₃, —(CF₂)₂CHF₂, —(CF₂)₃CHF₂, —(CF₂)₃CF₃, —CF(CF₃)₂ and —CF₂(CHF)CF₃,
S¹ has the same meaning and preferences as given above;
D has the same meaning as described above, and signifies preferably a group of formulae IIa, IIc, IId, IIe, IIf, IIg, IIh, IIi, IIj, IIk, if S¹ represents a single bond or a cyclic, straight-chain or branched, substituted or unsubstituted C₁-C₂₄alkylen and if S¹ represents a single bond or a cyclic, straight-chain or branched, substituted or unsubstituted C₂-C₂₄alkylen D is IIb, preferably D is a group of formulae IIa, IIj and IIk if S¹ represents a single bond or a cyclic, straight-chain or branched, substituted or unsubstituted C₁-C₂₄alkylen;
E represents an phenylene, an oxygen atom or a —N(H)— group;
S² represents a non-aromatic, aromatic, unsubstituted or substituted carbocyclic or heterocyclic group of formula (IV):

—(Z¹—C¹)ₐ₁—(Z²—C²)ₐ₂—(Z^{1a})ₐ₃— (IV)
wherein:
C¹, C² each independently represents a non-aromatic, aromatic, unsubstituted or substituted carbocyclic or heterocyclic group, and Z¹, Z², Z^{1a} each independently represents a bridging group, and a¹, a², a³ each independently represents an integer from 0 to 3, such that a¹+a²+a³≦6, preferably a¹+a²≦4 and a³ is 0;
wherein the bridging groups Z¹, Z^{1a} and Z² are as described above,
X, Y are hydrogen atoms, and
n is 1, 2 or 3, and n1 is 1 or 2; preferably n1 is 1;
   with the proviso that if n is 2 or 3 each A, B, x₁, D, E, S¹ and S² may be identical or different, and if n1 is 2 each B, x1 may be identical or different.

A more preferred embodiment of the present invention relates to diamine compounds (I), referring to any of the preceding definitions, and to alignment materials comprising these diamine compounds wherein
A represents a biphenylene, naphthylene or phenylene group, which is unsubstituted or mono- or poly-substituted by a halogen atom, a hydroxy group, and/or by acryloyloxy, and/or methacryloyloxy groups, and/or by straight-chain or branched alkyl, alkoxy, alkylcarbonyloxy, and/or alkyloxycarbonyl groups having from 1 to 20 carbon atoms,
   and
   wherein the compound residue (Ia) of compound of formula (I) as described in claim 1

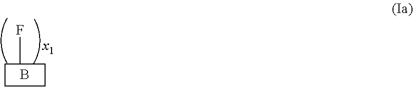

represents a straight-chain or branched C₁-C₈fluoralkyl group, wherein
F is fluorine, and
x₁ is an integer from 0 to 9,
B represents a straight-chain or branched C₁-C₈alkyl group, which is unsubstituted or in addition to its fluorine substituent(s) substituted by di-(C₁-C₁₆alkyl)amino, C₁-C₆alkyloxy, nitro, cyano and/or chlorine; and wherein one or more —CH₂— group may independently from each other be replaced by a linking group selected from —O—, —CO—, —CO—O—, —O—CO—, —NR¹—, —NR¹—CO—, —CO—NR¹— and —CH═CH—, wherein:
   R¹ represents a hydrogen atom or C₁-C₈alkyl;
   with the proviso that oxygen atoms are not directly linked to each other; and
S¹ has the same meaning and preferences as given above;
D has the same meaning as described above, and signifies preferably a group of formulae IIa, IIc, IId, IIe, IIf, IIg, IIh, IIi, IIj, IIk, if S¹ represents a single bond or a cyclic, straight-chain or branched, substituted or unsubstituted C₁-C₂₄alkylen and if S¹ represents a single bond or a cyclic, straight-chain or branched, substituted or unsubstituted C₂-C₂₄alkylen D is IIb, preferably D is a group of formulae IIa, IIj and IIk if S¹ represents a single bond or a cyclic, straight-chain or branched, substituted or unsubstituted C₁-C₂₄alkylen;
E represents an oxygen atom or a —N(H)— group;
S² represents a spacer unit of formula (IV) as defined above, wherein preferably S² is linked to A via Z¹; and wherein
   C¹, C² each independently represents a 1,4-phenylene which is unsubstituted or mono or poly-substituted by a halogen atom, and/or by an alkoxy, alkylcarbonyloxy or an alkyloxycarbonyl group, having form 1 to 10 carbon atoms, 1,4-cyclohexylene or a 4,4′-biphenylene group; and
Z¹, Z², Z^{1a} each independently represents —O—, —COO—, —OCO—, —CH₂—CH₂—, —OCH₂—, —CH₂O—, —CH═CH—, —C≡C—, —CH═CH—COO—, —OCO—CH═CH— or a single bond; and
a¹, a², a³ are independently 0 or 1; preferably a³ is 0,
n is 1 or 2, and n1 is 1, or 2, preferably 1;
   with the proviso that if n is 2 or 3 each A, B, x₁, D, E, S¹, S², X, Y may be identical or different; and if n1 is 2 each B, x₁ is identical or different.

Another preferred embodiment of the present invention relates to a diamine compound represented by one of formula (I), referring to any of the preceding definitions, and preferably to alignment materials comprising this diamine compound wherein
A represents 1,4-phenylene, which is unsubstituted or mono- or poly-substituted by a halogen atom, and/or by acryloyloxy or methacryloyloxy, and/or by an alkoxy, alkylcarbonyloxy or an alkyloxycarbonyl group, having from 1 to 10 carbon atoms, and
   wherein the compound residue (Ia) of compound of formula (I) as described above

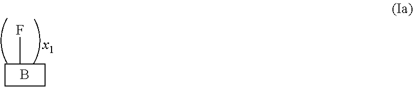

represents a straight-chain or branched C₁-C₈fluoralkyl group, wherein
F is fluorine, and
x₁ is an integer from 0 to 9,
B represents a straight-chain or branched C₁-C₈alkyl group, which is unsubstituted or in addition to its fluorine substituent(s) substituted by di-(C₁-C₁₆alkyl)amino, C₁-C₆alkyloxy, nitro, cyano and/or chlorine; and wherein one or more —CH₂— group may independently from each other be replaced by a linking group selected from —O—, —CO—, —CO—O—, —O—CO—, —NR¹—, —CO—NR¹— and —CH═CH—, wherein:
   R¹ represents a hydrogen atom or C₁-C₆alkyl;
   with the proviso that oxygen atoms are not directly linked to each other;
   and
   wherein the C₁-C₁₂fluoralkyl group has terminal units selected from —CF₂H and —CF₃, preferably selected from
   —CF₂H or —CF₃, —CF₂CF₃, —CF₂CHF₂, —(CF₂)₂CF₃, —(CF₂)₂CHF₂, —(CF₂)₃CHF₂, —(CF₂)₃CF₃, —CF(CF₃)₂ and —CF₂(CHF)CF₃,
D has the same meaning as described above, and signifies preferably a group of formulae IIa, IIc, IId, IIe, IIf, IIg, IIh, IIi, IIj, IIk, if S¹ represents a single bond or a cyclic, straight-chain or branched, substituted or unsubstituted C₁-C₂₄alkylen and if S¹ represents a single bond or a cyclic, straight-chain or branched, substituted or unsubstituted C₂-C₂₄alkylen D is IIb, preferably D is a group of formulae IIa, IIj and IIk if S¹ represents a single bond or a cyclic, straight-chain or branched, substituted or unsubstituted C₁-C₂₄alkylen;
   wherein
   Z⁴ is as defined above;
E represents an oxygen atom;
S¹ represents a single bond or a straight-chain C₁-C₈alkylene, preferably C₂-C₈alkylene group, wherein one —CH₂— group may be may be replaced by —O—, —COO—, —NR¹—CONR¹—, wherein R¹ is hydrogen or C₁-C₆alkyl, if D is a compound of formulae IIa, IIc, IId, IIe, IIf, IIg, IIh, IIi, IIj, IIk; and S¹
   represents a single bond or a cyclic, straight-chain or branched, substituted or unsubstituted C₅-C₂₄alkylen, preferably C₁₀-C₂₄alkylen, wherein one —CH₂— group may be may be replaced by, —COO—, if D is a compound of formula IIb;
represents a single bond or a straight-chain C₁-C₈alkylene group, wherein one —CH₂— group may be may be replaced by —O—, —OCO—, —COO—, —NR₁CO—, —CONR₁—, wherein R₁ is hydrogen or C₁-C₆alkyl,
S² is replaced by a group of formula (IVa) as described above,
   wherein:
   C¹ represents a 1,4-phenylene which is unsubstituted or mono or poly-substituted by a halogen atom, and/or by an alkoxy, alkylcarbonyloxy or an alkyloxycarbonyl group, having form 1 to 10 carbon atoms,
   Z¹, Z^{1a} represents —O—, —COO—, —OCO—, —COO(C₁-C₆)alkylen, —OCO(C₁-C₆)alkylen, —CH₂—CH₂—, —OCH₂—, —CH₂O—, —CH═CH—, —C≡C—, —CH═CH—COO—, —OCO—CH═CH—, or a single bond;
   X, Y are hydrogen atoms, and
   n is 1 or 2, and n1 is 1 or 2 and preferably 1;
with the proviso that if n or n1 is 2 each A, B, x₁, D, E, and S² may be identical or different; and if n1 is 2 each B, x₁ is identical or different.

Most preferred embodiment of the present invention relates to diamine compounds represented by one of the general formula (I), referring to any of the preceding definitions, and to alignment materials comprising these diamine compounds wherein
S² is replaced by a group of formula (IV), wherein:
C¹ represents 1,4-phenylene; and
Z¹, Z^{1a} represent each independently from each other —COO—, —OCO—, —CH₂—CH₂—, —OCH₂—, —CH₂O—, —CH═CH—, —C≡C—, —CH═CH—COO—, —OCO—CH═CH—, or a single bond;
a₁ represents 1, and a₃ represents 0
S² is linked to A via Z¹.
Especially most preferred embodiment of the present invention relates to Diamine compounds of formulae (VI), (VII), (VIII), (IX), (X), (XI), (XIa), (XIb), (XIc) and (XId)

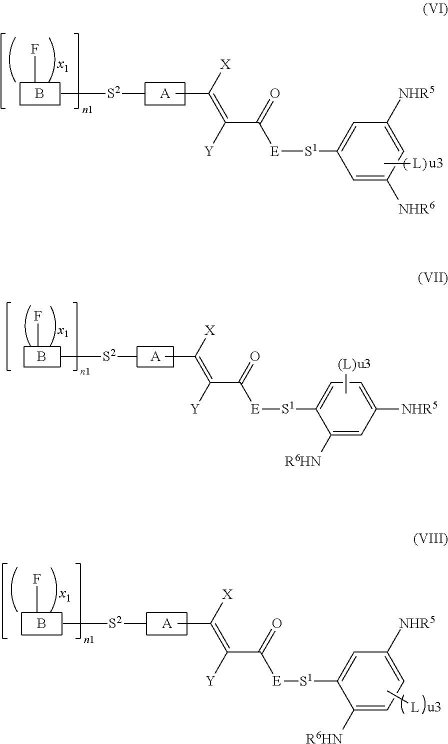

wherein in (VII) and (VIII) S¹ represents a single bond or a cyclic, straight-chain or branched, substituted or unsubstituted C₂-C₂₄alkylen, preferably C₅-C₂₄alkylen, more preferably C₁₀-C₂₄alkylen; the other substituents have the same meaning as given below for (VI), (IX), (X), (XI), (XIa), (XIb), (XIc) and (XId)

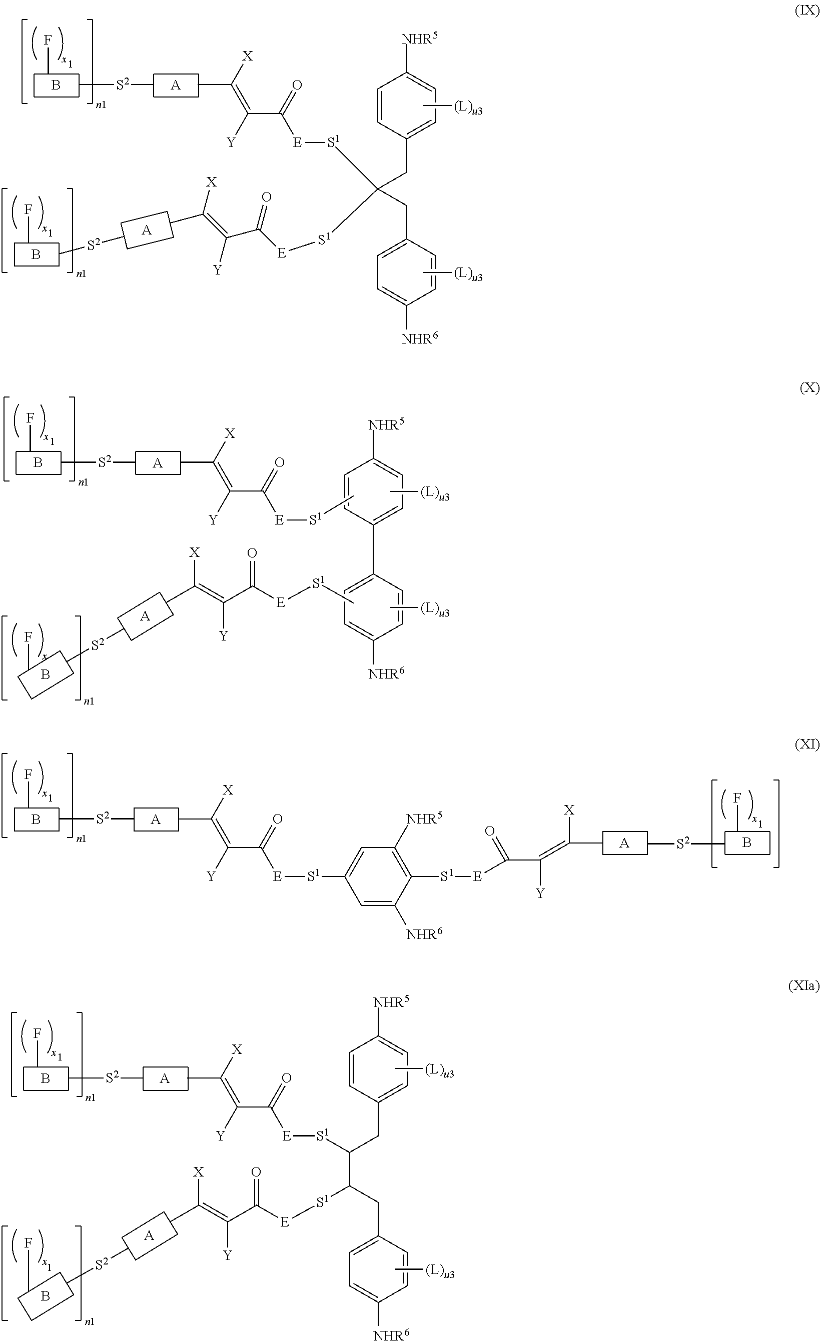

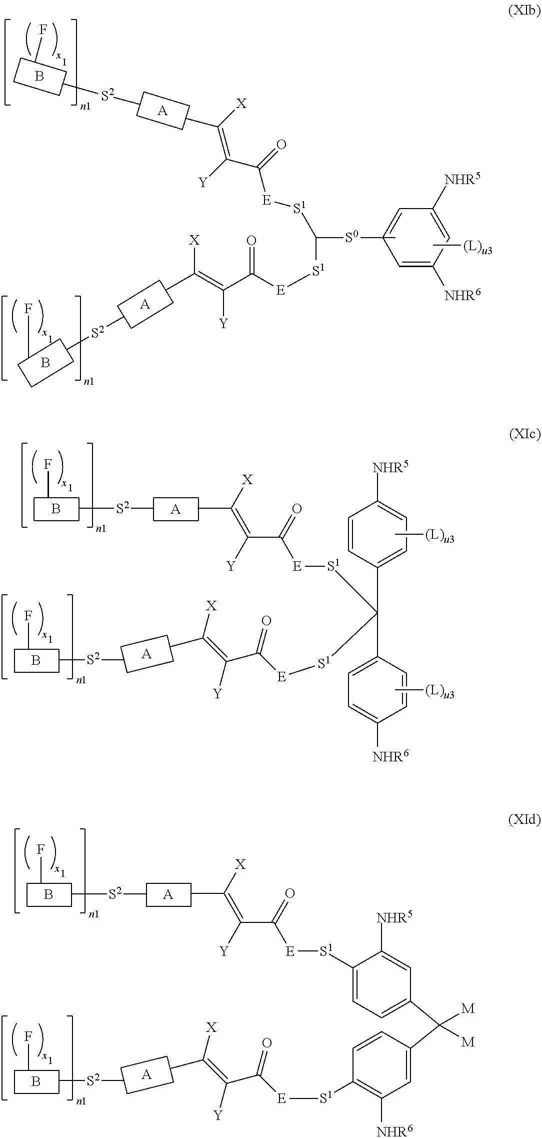

wherein
A, B, x₁, n, n1, D, E, M, S², S¹, S⁰, X and Y, R⁵, R⁶ and Z⁴ have the above given meanings and preferences as given above; preferably n1 is 1;
L is —CH₃, —OCH₃, —COCH₃, nitro, cyano, halogen, CH₂═CH—, CH₂═C(CH₃)—, CH₂═CH—(CO)O—, CH₂═CH—O—, CH₂═C(CH₃)—(CO)O—, or CH₂═C(CH₃)—O—,
u3 is an integer from 0 to 2;
and more especially most preferred diamine is a compound of formulae (VI), (IX) and (XIa) wherein A, B, x₁, n, n1, D, E, S², S¹, X and Y, R⁵, R⁶, Z⁴, L and u3 have the above given meanings and preferences as given above; or (VII), wherein S¹ represents a single bond or a cyclic, straight-chain or branched, substituted or unsubstituted C₂-C₂₄alkylen, preferably C₅-C₂₄alkylen, more preferably C₁₀-C₂₄alkylen;
and most especially most preferred diamine is a compound of formulae (VI), (IX) and (XIa) or (VII), wherein S¹ represents a single bond or a cyclic, straight-chain or branched, substituted or unsubstituted C₂-C₂₄alkylen, preferably C₅-C₂₄alkylen, more preferably C₁₀-C₂₄alkylen; especially that of formula (VI), wherein A, B, x₁, n, n1, D, E, S², X and Y, R⁵, R⁶, Z⁴, L and u₃ have the above given meanings and preferences as given above and S¹ represents a single bond or a straight-chain or branched C₁-C₁₄alkylen, wherein one or more, preferably non adjactent, —CH₂— group may independently be replaced by from —CO, —CO—O—, —CO—NR¹—, preferably by —CO—O—.

Further, especially most preferred embodiment of the present invention relates to diamine compounds of formula (XII)

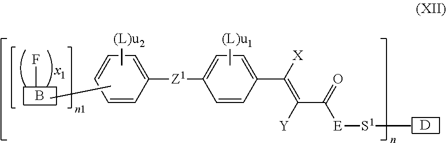

wherein x₁, n, n1, D, E, S¹, X, Y, Z¹, L, u₁ and u₂ have the above given meanings and preferences.

Preferred diamine compounds of formula (XII) are compounds, wherein Z¹ is —COO—, —OCO—, —OCO(C₁-C₆)alkylen or —COO(C₁-C₆)alkylen, or a single bond, or a straight-chain or branched, substituted or unsubstituted C₁-C₈alkylen, wherein one or more —CH₂— group may independently from each other be replaced independently from each other by a linking group, preferably by —O—.

Further, especially most preferred diamine is compound of formula (XIIa)

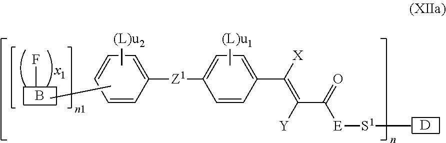

wherein n, n1, D, E, S¹, Z¹, L, u₁ and u₂ X and Y have the above given meanings and preferences as above, and
wherein the following compound residue

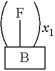

represents a straight-chain or branched C₁-C₈fluoralkyl group, wherein
F is fluorine, and
x₁ is an integer from 0 to 9,
B represents a straight-chain or branched C₁-C₈alkyl group, which is unsubstituted or in addition to its fluorine substituent(s) substituted by di-(C₁-C₁₆alkyl)amino, C₁-C₆alkyloxy, nitro, cyano and/or chlorine; and wherein one or more —CH₂— group may independently be replaced by a linking group selected from —O—, —CO—, —CO—O—, —O—CO—, —NR¹—, —NR¹—CO—, —CO—NR¹— and —CH═CH—, wherein:
   R¹ represents a hydrogen atom or C₁-C₈alkyl;
   with the proviso that oxygen atoms are not directly linked to each other;
   and
   wherein the C₁-C₁₂fluoralkyl group has terminal units selected from —CF₂H and —CF₃, preferably selected from
   —CF₂H or —CF₃, —CF₂CF₃, —CF₂CHF₂, —(CF₂)₂CF₃, —(CF₂)₂CHF₂, —(CF₂)₃CHF₂, —(CF₂)₃CF₃, —CF(CF₃)₂ and —CF₂(CHF)CF₃.

Most preferred diamine is

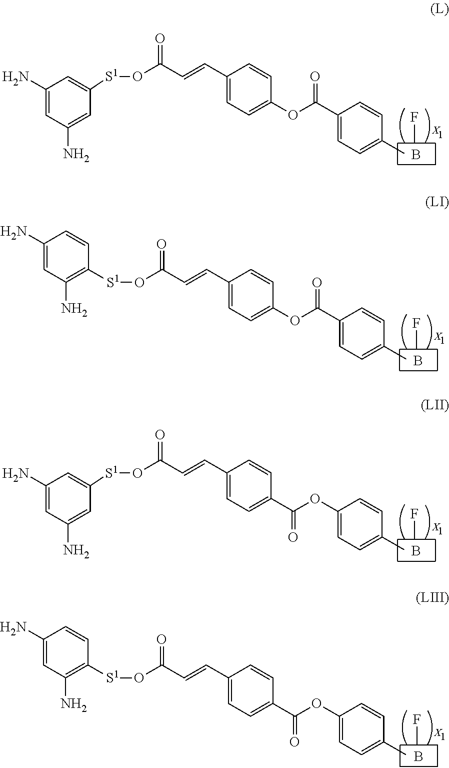

wherein for (L) and (LII) S¹ represents a single bond or a cyclic, straight-chain or branched, substituted or unsubstituted C₁-C₂₄alkylen; and wherein for (LI) and L(III) S¹ represents a single bond or a cyclic, straight-chain or branched, substituted or unsubstituted C₂-C₂₄alkylen, preferably C₅-C₂₄alkylen, more preferably C₁₀-C₂₄alkylen; especially most preferred for (L) and (LII) S¹ represents a straight-chain or branched C₁-C₁₂alkylen and for (LI) and (LIII) S¹ represents a straight-chain or branched C₂-C₁₂alkylen, wherein one or more —CH₂— group may be replaced by —O—, —O(CO)—, —(CO)O—, —NR¹CO—, —CONR₁—, wherein R₁ is hydrogen or C₁-C₆alkyl or a group of formula (IV), wherein:
C¹, C² each independently represents 1,4-phenylene; and
Z¹, Z², Z^{1a} each independently represents —COO—, —OCO—, —CH₂—CH₂—, —OCH₂—, —CH₂O—, —CH═CH—, —C≡C—, —CH═CH—COO, —OCO—CH═CH—, or a single bond; and
a₁, a₂, a₃ are independently 0 or 1, preferably a₃ is 0;
   and
   wherein the following compound residue

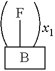

represents a straight-chain or branched C₁-C₈fluoralkyl group, wherein
F is fluorine, and
x₁ is an integer from 0 to 9,
B represents a straight-chain or branched C₁-C₈alkyl group, which is unsubstituted or in addition to its fluorine substituent(s) substituted by di-(C₁-C₁₆alkyl)amino, C₁-C₆alkyloxy, nitro, cyano and/or chlorine; and wherein one or more —CH₂— group may independently be replaced by a linking group selected from —O—, —CO—, —CO—O—, —O—CO—, —NR¹—, —NR¹—CO—, —CO—NR¹— and —CH═CH—, wherein:
   R¹ represents a hydrogen atom or C₁-C₆alkyl;
   with the proviso that oxygen atoms are not directly linked to each other;
   and
   wherein the C₁-C₈fluoralkyl group has terminal units selected from —CF₂H and —CF₃, preferably selected from
      —CF₂H or —CF₃, —CF₂CF₃, —CF₂CHF₂, —(CF₂)₂CF₃, —(CF₂)₂CHF₂, —(CF₂)₃CHF₂, —(CF₂)₃CF₃, —CF(CF₃)₂ and —CF₂(CHF)CF₃.

Another preferred embodiment of the present invention relates to diamine compounds represented by the general formula (I), which may be used in the subsequent manufacturing processes as such or in combination with one or more additional other diamine.

A further embodiment of the present invention is a composition comprising at least one diamine (I) and optionally at least one further diamine, which is different from (I) or/and an additive.

Additives such as silane-containing compounds and epoxy-containing crosslinking agents may be added.

Suitable silane-containing additives are described in Plast. Eng. 36 (1996), (Polyimides, fundamentals and applications), Marcel Dekker, Inc.

Suitable epoxy-containing cross-linking additives include 4,4′-methylene-bis-(N,N-diglycidylaniline), trimethylolpropane triglycidyl ether, benzene-1,2,4,5-tetracarboxylic acid 1,2,4,5-N,N′-diglycidyldiimide, polyethylene glycol diglycidyl ether, N,N-diglycidylcyclohexylamine and the like.

Additional additives are photo-sensitizers, photo-radical generators, cationic photo-initiators.

Suitable photo-active additives include 2,2-dimethoxyphenylethanone, a mixture of diphenylmethanone and N,N-dimethylbenzenamine or ethyl 4-(dimethylamino)-benzoate, xanthone, thioxanthone, Irgacure® 184, 369, 500, 651 and 907 (Ciba), Michler's ketone, triaryl sulfonium salt and the like.

Further the present invention relates to a process for the preparation of a diamine compound (XII) as defined above comprising contacting a compound of formula (XIV)

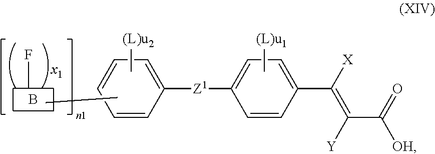

preferably

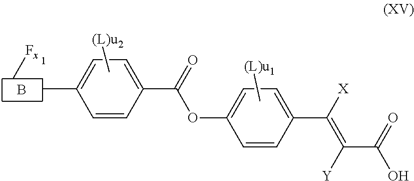

with a dinitro compound of formula (XVI)

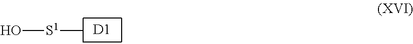

and then converting the obtained dinitro compound of formula (XVIa)

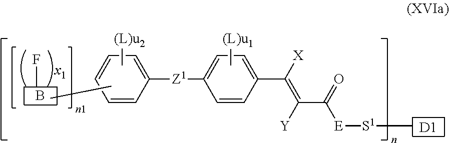

in the corresponding diamino compound of formula (XII)

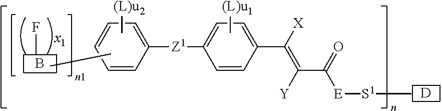

wherein F, x₁, n₁, n, B, D, X, Y, Z¹, L, u₁, u₂ and S¹ have the same meanings and x₁ is 0 or have the same meanings and preferences as given above, and wherein D1 has the same meaning and preferences as D as given above, with the proviso that the two amino groups of D are replaced by two nitro groups.

The reaction between compounds (XIV) and (XVI) can be conducted in many known ways (see J. March, Advanced Organic Chemistry, second edition, pages 363 and 365).

Usually, compounds (XIV) and (XVI) are contacted with a dehydrating agent. Commonly known dehydrating agents can be used. Preferred are EDC, 1-(3-dimethylaminopropyl)-3-ethylcarbodiimide hydrochloride or DCC, dicyclohexylcarbodiimide, trifluoroacetic anhydride, H₃BO₃—H₂SO₄, polymer-protected AlCl₃, pyridinium salts-Bu₃N or N,N-carbonyldiimidazole.

In general, the reaction of compounds (XIV) and (XVI) is conducted in a solvent. Usually organic solvents, such as for example toluene, xylene, pyridine, halogenalkane, such as dichlormethan, trichlorethan, acetone or dimethylformamide are used.

The conversion of nitro compounds to amino compounds is commonly known and for example described J. March, Advanced Organic Chemistry, 1977, pages 1125 and 1126). Further, the conversion can be conducted in analogy to the process described in WO 98/13331 and WO 96/36597.

Further, the present invention relates to compounds of formulae (XIV) and (XVI), and (XVIa) as given above.

In addition, the present invention relates to polymer, copolymer and oligomer comprising diamine (I′) or (I) as one of the basic building blocks.

Preferred polymer, copolymer and oligomer comprise diamine (I′) or (I) and a tetracarboxylic acid anhydride as basic building blocks.

Preferably, the polymer, copolymer or oligomer is comprising diamine (I′) or (I) as one basic building block are in the context of the invention a polyamic acid, polyamic ester, polyimide or a mixture thereof, preferably a mixture of polyamic acid and polyamic ester and/or polyimide. More preferred is a mixture of polyamic acid and polyimide.

In the context of the present invention the term “polyimide” has the meaning of partially or complete imidisated polyamic acid or polyamic ester. In analogy, the term “imidisation” has in the context of the present invention the meaning of partially or complete imidisation.

Preferably, the tetracarboxylic acid anhydride is of formula (V)

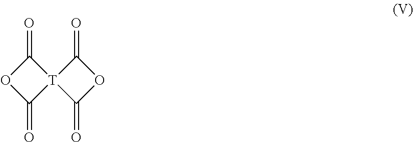

wherein:
T represents a tetravalent organic radical.

The tetravalent organic radical T is preferably derived from an aliphatic, alicyclic or aromatic tetracarboxylic acid dianhydride.

Preferred examples of aliphatic or alicyclic tetracarboxylic acid dianhydrides are:
1,1,4,4-butanetetracarboxylic acid dianhydride,
ethylenemaleic acid dianhydride,
1,2,3,4-cyclobutanetetracarboxylic acid dianhydride,
1,2,3,4-cyclopentanetetracarboxylic acid dianhydride,
2,3,5-tricarboxycyclopentylacetic acid dianhydride,
tetrahydro-4,8-methanofuro[3,4-d]oxepine-1,3,5,7-tetrone,
3-(carboxymethyl)-1,2,4-cyclopentanetricarboxylic acid 1,4:2,3-dianhydride,
hexahydrofuro[3′,4′:4,5]cyclopenta[1,2-c]pyran-1,3,4,6-tetrone,
3,5,6-tricarboxynorbornylacetic acid dianhydride,
2,3,4,5-tetrahydrofurantetracarboxylic acid dianhydride,
rel-[1S,5R,6R]-3-oxabicyclo[3.2.1]octane-2,4-dione-6-spiro-3′-(tetrahydrofuran2′,5′-dione),
4-(2,5-dioxotetrahydrofuran-3-yl)tetrahydronaphthalene-1,2-dicarboxylicacid dianhydride,
5-(2,5-dioxotetrahydrofuran-3-yl)-3-methyl-3-cyclohexene-1,2-dicarboxylic-acid dianhydride,
bicyclo[2.2.2]oct-7-ene-2,3,5,6-tetracarboxylic acid dianhydride,
bicyclo[2.2.2]octane-2,3,5,6-tetracarboxylic acid dianhydride,
1,8-dimethylbicyclo[2.2.2]oct-7-ene-2,3,5,6-tetracarboxylic acid dianhydride, pyromellitic acid dianhydride,
3,3′,4,4′-benzophenonetetracarboxylic acid dianhydride,
4,4′-oxydiphthalic acid dianhydride,
3,3′,4,4′-diphenylsulfonetetracarboxylic acid dianhydride,
1,4,5,8-naphthalenetetracarboxylic acid dianhydride,
2,3,6,7-naphthalenetetracarboxylic acid dianhydride,
3,3′,4,4′-dimethyldiphenylsilanetetracarboxylic acid dianhydride,
3,3′,4,4′-tetraphenylsilanetetracarboxylic acid dianhydride,
1,2,3,4-furantetracarboxylic acid dianhydride,
4,4′-bis(3,4-dicarboxyphenoxy)diphenyl sulfide dianhydride,
4,4′-bis(3,4-dicarboxyphenoxy)diphenyl sulfone dianhydride,
4,4′-bis(3,4-dicarboxyphenoxy)diphenylpropane dianhydride,
3,3′,4,4′-biphenyltetracarboxylic acid dianhydride,
ethylene glycol bis(trimellitic acid) dianhydride,
4,4′-(1,4-phenylene)bis(phthalic acid) dianhydride,
4,4′-(1,3-phenylene)bis(phthalic acid) dianhydride,
4,4′-(hexafluoroisopropylidene)diphthalic acid dianhydride,
4-tert-butyl-6-(2,5-dioxotetrahydro-3-furanyl)-2-benzofuran-1,3-dione,
5-(2,5-dioxotetrahydro-3-furanyl)-3a,4,5,9b-tetrahydronaphtho[1,2-c]furan-1,3-dione,
5-(2,5-dioxotetrahydro-3-furanyl)-5-methyl-3a,4,5,9b-tetrahydronaphtho[1,2-c]furan-1,3-dione,
5-(2,5-dioxotetrahydro-3-furanyl)-6-methylhexahydro-2-benzofuran-1,3-dione,
5-(2,5-dioxotetrahydro-3-furanyl)-7-methyl-3a,4,5,7a-tetrahydro-2-benzofuran-1,3-dione,
6-(2,5-dioxotetrahydro-3-furanyl)-4-methylhexahydro-2-benzofuran-1,3-dione,
9-isopropyloctahydro-4,8-ethenofuro[3′,′:3,4]cyclobuta[1,2-f][2]benzofuran-1,3,5,7-tetrone,
1,2,5,6-cyclooctanetetracarboxylic acid dianhydride,
octahydro-4,8-ethenofuro[3′,′:3,4]cyclobuta[1,2-f][2]benzofuran-1,3,5,7-tetrone,
octahydrofuro[3′,4′:3,4]cyclobuta[1,2-f][2]benzofuran-1,3,5,7-tetrone,
tetrahydro-3,3′-bifuran-2,2′,5,5′-tetrone,
4,4′-oxydi(1,4-phenylene)bis(phthalic acid) dianhydride, and
4,4′-methylenedi(1,4-phenylene)bis(phthalic acid) dianhydride.

Preferred examples of aromatic tetracarboxylic acid dianhydrides are:
pyromellitic acid dianhydride,
3,3′,4,4′-benzophenonetetracarboxylic acid dianhydride,
4,4′-oxydiphthalic acid dianhydride,
3,3′,4,4′-diphenylsulfonetetracarboxylic acid dianhydride,
1,4,5,8-naphthalenetetracarboxylic acid dianhydride,
2,3,6,7-naphthalenetetracarboxylic acid dianhydride,
3,3′,4,4′-dimethyldiphenylsilanetetracarboxylic acid dianhydride,
3,3′,4,4′-tetraphenylsilanetetracarboxylic acid dianhydride,
1,2,3,4-furantetracarboxylic acid dianhydride,
4,4′-bis(3,4-dicarboxyphenoxy)diphenyl sulfide dianhydride,
4,4′-bis(3,4-dicarboxyphenoxy)diphenyl sulfone dianhydride,
4,4′-bis(3,4-dicarboxyphenoxy)diphenylpropane dianhydride,
3,3′,4,4′-biphenyltetracarboxylic acid dianhydride,
ethylene glycol bis(trimellitic acid) dianhydride,
4,4′-(1,4-phenylene)bis(phthalic acid) dianhydride,
4,4′-(1,3-phenylene)bis(phthalic acid) dianhydride,
4,4′-(hexafluoroisopropylidene)diphthalic acid dianhydride,
4,4′-oxydi(1,4-phenylene)bis(phthalic acid) dianhydride,
4,4′-methylenedi(1,4-phenylene)bis(phthalic acid) dianhydride,
4-tert-butyl-6-(2,5-dioxotetrahydro-3-furanyl)-2-benzofuran-1,3-dione, and the like.

More preferably the tetracarboxylic acid dianhydrides used to form the tetravalent organic radical T are selected from:
1,2,3,4-cyclobutanetetracarboxylic acid dianhydride,
1,2,3,4-cyclopentanetetracarboxylic acid dianhydride,
2,3,5-tricarboxycyclopentylacetic acid dianhydride,
tetrahydro-4,8-methanofuro[3,4-d]oxepine-1,3,5,7-tetrone,
3-(carboxymethyl)-1,2,4-cyclopentanetricarboxylic acid 1,4:2,3-dianhydride,
hexahydrofuro[3′,4′:4,5]cyclopenta[1,2-c]pyran-1,3,4,6-tetrone,
5-(2,5-dioxotetrahydrofuran-3-yl)-3-methyl-3-cyclohexene-1,2-dicarboxylic acid dianhydride,
pyromellitic acid dianhydride,
4-(2,5-dioxotetrahydrofuran-3-yl)tetrahydronaphthalene-1,2-dicarboxylic acid dianhydride,
5-(2,5-dioxotetrahydro-3-furanyl)-5-methyl-3a,4,5,9b-tetrahydronaphtho[1,2-c]furan-1,3-dione,
5-(2,5-dioxotetrahydro-3-furanyl)-3a,4,5,9b-tetrahydronaphtho[1,2-c]furan-1,3-dione,
5-(2,5-dioxotetrahydro-3-furanyl)-7-methyl-3a,4,5,7a-tetrahydro-2-benzofuran-1,3-dione,
4-tert-butyl-6-(2,5-dioxotetrahydro-3-furanyl)-2-benzofuran-1,3-dione,
4,4′-(hexafluoroisopropylidene)diphthalic acid dianhydride and bicyclo[2.2.2]oct-7-ene-2,3,5,6-tetracarboxylic acid dianhydride.

The polymer, copolymer or oligomer, especially the polyamic acid, polyamic acid ester and polyimide and mixtures thereof may be prepared in line with known methods, such as those described in Plast. Eng. 36 (1996), (Polyimides, fundamentals and applications), Marcel Dekker, Inc.

For example, the amidisation, poly-condensation reaction for the preparation of the polyamic acids is carried out in solution in a polar aprotic organic solvent, such as γ-butyrolactone, N,N-dimethylacetamide, N-methylpyrrolidone or N,N-dimethyl-formamide. In most cases equimolar amounts of the anhydride and the diamine are used, i.e. one amino group per anhydride group. If it is desired to stabilize the molecular weight of the polymer, copolymer or oligomer, it is possible for that purpose to either add an excess or a less-than-stoichiometric amount of one of the two components or to add a mono-functional compound in the form of a dicarboxylic acid monoanhydride or in the form of a monoamine. Examples of such mono-functional compounds are maleic acid anhydride, phthalic acid anhydride, aniline and the like. Preferably the reaction is carried out at temperatures of less than 100° C.

The imidisation, cyclisation of the polyamic acids to form the polyimides can be carried out by heating, i.e. by condensation with removal of water or by other imidisation reactions using appropriate reagents.

Partially imidisation is achieved for example, if the imidisation is carried out purely thermally, the imidisation of the polyamic acids may not always be complete, i.e. the resulting polyimides may still contain proportions of polyamic acid.

Complete imidisation reactions are carried out at temperatures between 60 and 250° C., preferably at temperatures of less than 200° C.

In order to achieve imidisation at lower temperatures additional reagents that facilitate the removal of water are added to the reaction mixture. Such reagents are, for example, mixtures consisting of acid anhydrides, such as acetic acid anhydride, propionic acid anhydride, phthalic acid anhydride, trifluoroacetic acid anhydride or tertiary amines, such as triethylamine, trimethylamine, tributylamine, pyridine, N,N-dimethylaniline, lutidine, collidine etc. The amount of aforementioned additional reagents that facilitate the removal of water is preferably at least four equivalents of acid anhydride and two equivalents of amine per equivalent of polyamic acid to be condensed.

The imidization degree of each polymer used in the liquid crystal alignment agent of the invention can be arbitrarily adjusted by controlling the catalyst amount, reaction time and reaction temperature employed in production of the polymer. In the present description, “imidization degree” of polymer refers to a proportion (expressed in %) of the number of recurring units of polymer forming an imide ring or an isoimide ring to the number of total recurring units of polymer. In the present description, the imidization degree of a polyamic acid not subjected to dehydration and ring closure is 0%. The imidization degree of each polymer is determined by dissolving the polymer in deuterated dimethyl sulfoxide, subjecting the resulting solution to ¹H-NMR measurement at a room temperature using tetramethylsilane as a standard substance, and calculating from the following formula.

Imidization degree (%)=1−(A¹/A2×B)×100

A¹: Peak area based on protons of NH groups (in the vicinity of 10 ppm)
A²: Peak area based of one proton of acrylate double bond (in the vicinity of 6.5 ppm).
B: Proportion of the number of acrylate protons to one proton of NH group in the polymer precursor

The imidization degree is usually in the range of 1 to 99%, preferably 5 to 50%, more preferably 10 to 40%.

The present invention concerns a process for the preparation of a polymer, copolymer or oligomer comprising polymerisation of a diamine (I′) or (I).

Preferably the polymerisation of a diamine (I′) or (I) comprises
a) amidisation of at least one diamine (I′) or (I) to polyamic acid or a polyamic ester, and
b) imidisation of the obtained polyamic acid or ester, to a polyimide, or
c) imidisation of the diamine (I′) or (I) to polyimide.

In a more preferred embodiment of the invention, the polymersiation of the diamine comprises the amidsation of at least one diamine (I′) or (I) with tetracarboxylic acid anhydride, preferably tetracarboxylic acid anhydride (V), and/or the imidisation, preferably by elevated temperature.

In a further more preferred embodiment of the invention, the polymersiation of the diamine comprises the amidsation of a diamine (I′) or (I) with tetracarboxylic acid anhydride, preferably tetracarboxylic acid anhydride (V), and/or the imidisation, preferably by elevated temperature, and wherein the amidisation and/or imidisation is optionally conducted
in the presence of additives as given above, and/or
in the presence of a further diamine, which is different from that of formula (I′) or (I), preferably in the presence of at least one diamine (L) and/or
in the presence of a further polymer, copolymer or oligomer comprising as one basic building block a diamine (L), or a further polymer, copolymer or oligomer, which is different from a polyamic acid, polyamic ester or a polyimide, more preferably a further polymer, copolymer or oligomer, which is selected from the group of which is selected from the group of polymers include polyacrylates, polymethacrylates, polyacrylamides, polymethacrylamides, polyvinylether and polyvinylester, polyallylether and ester, polystyrenes, polysiloxanes, polyimides, polyamic acids and their esters, polyamidimides, polymaleic acids, polyfumaric acids polyurethanes and derivatives thereof.

Preferably, the further polymer, copolymer or oligomer comprises as basic building block a diamine (L) and a tetracarboxylic acid anhydride, preferably a tetracarboxylic acid anhydride of formula (V).

This polymer, copolymer or oligomer is prepared in analogy to the polymer, copolymer or oligomer of the invention comprising diamine (I′) or (I).

The imididation is conducted after or during amidisation. In general, the imidisation is conducted after amidisation.

Preferred is the partially imidisation of polyamic acid or polyamic ester.

If the polymer is prepared only by imidisation, diamine (I′) or (I) will be contacted with an imidisation compound, with at least two polymerisable functional groups, such as for example, carbonyl groups or halogen groups.

More preferably, the present invention concerns a process for the preparation of a polymer, copolymer or oligomer comprising polymerisation of a diamine (I′) or (I) and tetracarboxylic acid anhydride, preferably tetracarboxylic acid anhydride (V).

Another embodiment of the present invention relates to a copolymer comprising diamine (I). Preferred is a copolymer, comprising at least two diamines (I).

A further embodiment of the present invention relates to a polymer, copolymer or oligomer, or to blends obtainable according to the processes and preferred processes of the invention.

Preferably, blends are obtainable by reaction of at least two different diamine (I′) or (I), or by reaction of at least one diamine (I′) or (I) with a polymer, copolymer or oligomer comprising as basic building block at least one diamine (L).

Preferably, the present invention concerns polymer, copolymer or oligomer, comprising in their polymer-, copolymer- or oligomer-side-chains at least one photo-reactive group. Preferably, the photo-reactive group of the side chains are photo-isomerized and/or crosslinked, more preferably photo-dimerized, by exposure to aligning light.

The term photoreactive groups have the meaning of groups, which are able to react by interaction with light.

The treatment with aligning light may be conducted in a single step or in several seperate steps. In a preferred embodiment of the invention the treatment with aligning light is conducted in a single step.

In the context of the present invention photo-reactive group has preferably the meaning of a dimerizable, isomerizable, polymerizable and/or cross-linkable group.

In the context of the present invention, aligning light is light of wavelengths, which can initiate photoalignment. Preferably, the wavelengths are in the UV-A, UVB and/or UV/C-range, or in the visible range. It depends on the photoalignment compound, which wavelengths are appropriate. Preferably, the photo-reactive groups are sensitive to visible and/or UV light. A further embodiment of the invention concerns the generating of aligning light by laser light.

The instant direction of the aligning light may be normal to the substrate or at any oblique angle.

For generating tilt angles, preferably the aligning light is exposed from oblique angles.

More preferably, aligning light is at least partially linearly polarized, elliptically polarized, such as for example circulary polarized, or non-polarized; most preferably at least circulary or partially linearly polarized light, or non-polarized light exposed obliquely. Especially, most preferred aligning light denotes substantially polarised light, especially linearly polarised light; or aligning light denotes non-polarised light, which is applied by an oblique irradiation.

In a more preferred embodiment of the invention the polymer, copolymer or oligomer is treated with polarised light, especially linearly polarised light, or by oblique radiation with non-polarised light.

Further preferred are polymers, copolymers or oligomers of the present invention,
wherein at least 30%, preferably at least 75% of the repeating units include a side chain with a photo-reactive group; and/or
wherein, the photo-reactive groups are able to undergo photo-dimerisation, preferably photo-cyclisation, in particular [2+2]-photo-cyclisation; and/or
wherein the polymer or oligomer is a polymer gel or a polymer network, or an oligomer gel or an oligomer network, respectively; and/or
wherein the polymer, copolymer or oligomer has an intrinsic viscosity in the range of 0.05 to 10 dL/g, preferably in the range of 0.05 to 5 dL/g; and/or
wherein the polymer, copolymer or oligomer contains from 2 to 2000 repeating units, especially from 3 to 200 repeating units; and/or
wherein the polymer, copolymer or oligomer is in the form of a homopolymer or of a copolymer, preferably of a statistical copolymer; and/or
wherein the polymer, copolymer or oligomer is cross-linkable or cross-linked;

A further preferred embodiment of the present invention concerns polymers, copolymers or oligomers, having an intrinsic viscosity preferably in the range of 0.05 to 10 dL/g, more preferably in the range of 0.05 to 5 dL/g. Herein, the intrinsic viscosity (ηᵢₙₕ=ln ηᵣₑₗ/C) is determined by measuring a solution containing a polymer or an oligomer in a concentration of 0.5 g/100 ml solution for the evaluation of its viscosity at 30° C. using N-methyl-2-pyrrolidone as solvent.

In addition, a preferred embodiment of the present invention concerns polymers, copolymers or oligomers, containing from 2 to 2000 repeating units, especially from 3 to 200 repeating units.

The side-chain polymers or oligomers according the invention can be present in the form of homopolymers as well as in the form of copolymers. The term “copolymers” is to be understood as meaning especially statistical copolymers.

Further, the present invention concerns a composition, especially a blend, comprising
a polymer, copolymer or oligomer according to definition and preferences of the invention, comprising at least a diamine (I′) or (I) as basic building block, or
a polymer, copolymer or oligomer according to definition and preferences of the invention, obtainable by the processes of the invention, and
preferably in addition comprising and a further diamine, which is different from diamine (I′) or (I), preferably a diamine (L).

The further polymer, copolymer or oligomer comprising as one basic building block a diamine (L) has the same preferences as given above.

Preferably, the present invention concerns a composition, especially a blend, comprising
a polymer, copolymer or oligomer according to definition and preferences of the invention, comprising at least a diamine (I′) or (I) as basic building block, or
a polymer, copolymer or oligomer according to definition and preferences of the invention, obtainable by the processes of the invention,
and/or a further polymer, copolymer or oligomer comprising as one basic building block a further diamine, which is different from diamine (I′) or (I), preferably a diamine (L), or a further polymer, copolymer or oligomer, which is different from a polyamic acid, polyamic ester or a polyimide, more preferably a further polymer, copolymer or oligomer, which is selected from the group of polyacrylate, polystyrol, polyester, polyurethane, polyethylene, poylpopylen, polyvinylchloride, polytetrafluoroethylen, polycabonate, polyterephthalate and dendrimere.

Further preferably, the present invention concerns a composition, especially a blend, comprising
a polymer, copolymer or oligomer according to definition and preferences of the invention, comprising at least a diamine (I′) or (I) as basic building block, or
a polymer, copolymer or oligomer according to definition and preferences of the invention, obtainable by the processes of the invention,
and optionally a further diamine, which is different from diamine (I′) or (I), preferably a diamine (L),
and an additive, preferably silane-containing compounds,
and/or a further polymer, copolymer or oligomer comprising as one basic building block a further diamine, which is different from diamine (I), preferably at least one diamine (L),
and/or a further polymer, copolymer or oligomer, which is different from a polyamic acid, polyamic ester or a polyimide, more preferably a further polymer, copolymer or oligomer, which is selected from the group of polymers include polyacrylates, polymethacrylates, polyacrylamides, polymethacrylamides, polyvinylether and polyvinylester, polyallylether and ester, polystyrenes, polysiloxanes, polyimides, polyamic acids and their esters, polyamidimides, polymaleic acids, polyfumaric acids polyurethanes and derivatives thereof,
and/or photo-active polymers, photo-active oligomers and/or photo-active monomers,
and/or cross-linking agents, preferably epoxy-containing cross-linking agents, most preferably selected from the group:
4,4′-methylene-bis-(N,N-diglycidylaniline), trimethylolpropane triglycidyl ether, benzene-1,2,4,5-tetracarboxylic acid 1,2,4,5-N,N′-diglycidyldiimide, polyethylene glycol diglycidyl ether, N,N-diglycidylcyclohexylamine.

The polymers or oligomers according to the invention may be used in form of polymer layers or oligomer layers alone or in combination with other polymers, oligomers, monomers, photo-active polymers, photo-active oligomers and/or photo-active monomers, depending upon the application to which the polymer or oligomer layer is to be added. Therefore it is understood that by varying the composition of the polymer or oligomer layer it is possible to control specific and desired properties, such as an induced pre-tilt angle, good surface wetting, a high voltage holding ratio, a specific anchoring energy, etc.

Polymer or oligomer layers may readily be prepared from the polymers or oligomers of the present invention and a further embodiment of the invention relates to a polymer or oligomer layer comprising a polymer or oligomer according to the present invention, which is preferably prepared by treatment with aligning light. Preferably, the invention relates to a polymer or oligomer layer comprising a polymer or oligomer according to the present invention in a cross-linked and/or isomerized form.

The polymer or oligomer layer is preferably prepared by applying one or more polymers or oligomers according to the invention to a support and, after imidisation or without imidisation, treating, preferably cross-linking and for isomerising, the polymer or oligomer or polymer mixture or oligomer mixture by irradiation with aligning light.

In general, Si wafer or a transparent support such as glass or plastic substrates, optionally coated with indium tin oxide (ITO) are used.

A further preferred embodiment of the present invention relates to a polymer, copolymer or oligomer layer, comprising at least one polymer, copolymer or oligomer according to the present invention and preferably on a Si wafer.

Further, it is possible to vary the direction of orientation and the tilt angle within the polymer or oligomer layer by controlling the direction of the irradiation of the aligning light. It is understood that by selectively irradiating specific regions of the polymer or oligomer layer very specific regions of the layer can be aligned. In this way, layers with a defined tilt angle can be provided. The induced orientation and tilt angle are retained in the polymer or oligomer layer by the process, especially by the process of cross-linking.

Method for the preparation of a polymer, copolymer or oligomer according to the invention, wherein in a polycondensation reaction a diamine (I′) or (I) is reacted with one or more tetracarboxylic acid anhydrides of the general formula (V), optionally in the presence of one or more additional other diamines.

Further, the present invention preferably concerns a method, wherein a poly-condensation reaction for the preparation of the polyamic acids is carried out in solution in a polar aprotic organic solvent, preferably selected from γ-butyrolactone, N,N-dimethylacetamide, N-methylpyrrolidone or N,N-dimethylformamide.

Preferably, the present invention concerns a method, wherein subsequent to the poly-condensation cyclisation with removal of water is carried out thermally under formation of a polyimide.

More preferably, the present invention concerns a method, wherein imidisation is carried out prior or after the application of the polymer, copolymer or oligomer to a support.

Further preferred methods of the invention relates to
a method for the preparation of a polymer layer or oligomer layer, which are vertically aligned;
a method for the preparation of multi-domain vertical alignment of a polymer layer or oligomer layer;
a method for the preparation of a polymer layer or oligomer layer with tilted optical axis.

A further embodiment of the present invention concerns a polymer, copolymer or oligomer layer, in particular orientation layer, comprising at least one polymer, copolymer or oligomer according to the present invention.

It is understood that the polymer or oligomer layers of the present invention (in form of a polymer gel, a polymer network, a polymer film, etc.) can also be used as orientation layers for liquid crystals. A further preferred embodiment of the invention relates to an orientation layer comprising one or more polymers or oligomers according to the invention, preferably in a cross-linked form. Such orientation layers can be used in the manufacture of unstructured or structured optical- or electro-optical elements, preferably in the production of hybrid layer elements.

In addition, the present invention concerns a method for the preparation of a polymer layer or oligomer layer, wherein one or more polymers, copolymers or oligomers according to the present invention is applied to a support, preferably from a solution of the polymer or oligomer material and subsequent evaporation of the solvent, and wherein, after any imidisation step which may be necessary, the polymer or oligomer or polymer mixture or oligomer mixture treated with aligning light, and preferably isomerized and/or cross-linked by irradiation with aligning light.

A preferred method of the present invention concerns a method, wherein the direction of orientation and the tilt angle within the polymer layer or oligomer layer is varied by controlling the direction of the irradiation with aligning light, and/or wherein by selectively irradiating specific regions of the polymer layer or oligomer layer specific regions of the layer are aligned.

The orientation layers are suitably prepared from a solution of the polymer or oligomer material. The polymer or oligomer solution is applied to a support optionally coated with an electrode [for example a glass plate coated with indium-tin oxide (ITO)] so that homogeneous layers of 0.05 to 50 μm thickness are produced. In this process different coating techniques like spin-coating, meniscus-coating, wire-coating, slot-coating, offset-printing, flexo-printing, gravur-printing may be used. Then, or optionally after a prior imidisation step, the regions to be oriented are irradiated, for example, with a high-pressure mercury vapour lamp, a xenon lamp or a pulsed UV laser, using a polarizer and optionally a mask for creating images of structures.

Further, the present invention concerns the use of a polymer layer, copolymer or oligomer layer according to the present invention, preferably in cross-linked form, as an orientation layer for liquid crystals.

Further, the present invention concerns preferably the use of a polymer layer, copolymer or oligomer layer for the induction of vertical alignment of adjacent liquid crystalline layers, in particular for operating a cell in MVA mode.

The irradiation time is dependent upon the output of the individual lamps and can vary from a few seconds to several hours. The photo-reaction (dimerisation, polymersiation, cross-linking) can also be carried out, however, by irradiation of the homogeneous layer using filters that, for example, allow only the radiation suitable for the cross-linking reaction to pass through.

It is understood that the polymer or oligomer layers of the invention may be used in the production of optical or electro-optical devices having at least one orientation layer as well as unstructured and structured optical elements and multi-layer systems.

The present invention concerns the use of a polymer layer, copolymer or oligomer layer as an orientation layer for liquid crystals.

Preferred is the use for the induction of vertical alignment of adjacent liquid crystalline layers.

A further embodiment of the invention relates to an optical or electro-optical device comprising one or more polymers or oligomers according to the present invention in cross-linked form. The electro-optical devices may comprise more than one layer. The layer, or each of the layers may contain one or more regions of different spatial orientation.

Preferably, the present invention concerns an optical and electro-optical unstructured or structured constructional elements, preferably liquid crystal display cells, multi-layer and hybrid layer elements, comprising at least one polymer layer, copolymer or oligomer layer according to the present invention.

More preferably, the present invention concerns an orientation layer, comprising at least one polymer layer, copolymer or oligomer layer according to the present invention.

It was also found that with the diamines (I′) and (I) and their polymers good values of Voltage Holding Ratio (VHR), Residual DC (RDC) or AC Memory (ACM) are accessible.

VHR, ACM and RDC are commonly known values in the technical field of liquid crystal displays and will be described as following:

### VHR:

In the case of liquid crystal displays of thin-film transistor type a certain amount of charge is applied over the course of a very short period of time to the electrodes of a pixel and must not subsequently drain away by means of the resistance of the liquid crystal. The ability to hold that charge and thus to hold the voltage drop over the liquid crystal is quantified by what is known as the “voltage holding ratio” (VHR). It is the ratio of the RMS-voltage (root mean square voltage) at a pixel within one frame period and the initial value of the voltage applied.

ACM (AlternativeCurrentMemory): An AC (AlternativeCurrent) voltage of 7 Volts (1 kHz) is applied to the cell for 700 hours. The pre-tilt angle of the cell is measured before and after the application of the AC stress. The ACM performance is expressed in terms of a pretilt angle difference, Δθ.

RDC (ResidualDirectCurrent): An adjustable DC (DirectCurrent)-component of V=2VDC is added to the symmetric square wave signal of V=2.8V (30 Hz), the fluctuations of light transmitted by the test cell can be eliminated or at least minimized by adequate selection of the external DC component. The external DC-voltage for which the flicker is eliminated or minimized by compensation of the internal residual DC-voltage is taken to be equivalent to the internal residual DC-voltage.

The advantages of the present invention could not be foreseen by a skilled person. It has surprisingly been found, that in addition to the polyamic/polyimide backbone, the introduction of an organofluorine group into a peripheral position of the polymer side groups having specific molecular architecture plays a predominant role in obtaining MVA materials having optimised properties, such as the required high voltage holding ratios, the adjustable pre-tilt angles required for the MVA mode and their stability to light and heat.

### EXAMPLE 1

### Synthesis

### Preparation of (2E)-3-(4-{[4-(4,4,4-trifluorobutoxy)benzoyl]oxy}phenyl)acrylic acid

### 1.1 Preparation of 4-(4,4,4-trifluorobutoxy)benzoic acid

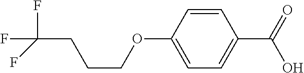

55.00 g (0.408 Mol) 4,4,4-trifluorobutan-1-ol are dissolved in 550 ml tetrahydrofurane, 142 ml (0.102 Mol) triethylamine are added at room temperature. 38 ml (0.490 Mol) methanesulfonyl chloride were added dropwise under nitrogen. The mixture is stirred for 1 h at 0-5° C. The beige suspension is Hyflo-filtrated and washed with tetrahydrofurane. The filtrate is concentrated. The residue is dissolved in 1.4 l 1-methyl-2-pyrrolidone 62.70 g (0.408 Mol) of methyl 4-hydroxybenzoate and 226.00 g (1.43 Mol) of potassium carbonate are added to the lightly brown solution. The reaction suspension is allowed to react at 80° C. for 14 h. 1 l (1.0 Mol) of a 1N NaOH solution is added to the above mixture. The suspension is heated at reflux temperature for 30 min until the reaction is completed. The reaction mixture is allowed to cool at room temperature and thrown in cold water. The solution is carefully acidified with a 25% HCl solution and is stirred for 15 min. The product is filtrated off, washed with water and dried overnight at room temperature under vacuum to give 99.00 g (98%) of 4-(4,4,4-trifluorobutoxy)benzoic acid as a white solid.

### 1.2 Preparation of 4-Formylphenyl-4-(4,4,4-trifluorobutoxy)benzoate

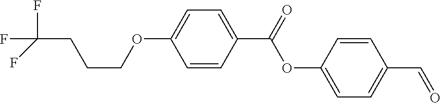

6.89 g (56.4 mmol) of 4-hydroxybenzaldehyd, 14.0 g (56.4 mmol) of 4-(4,4,4-trifluorobutoxy)benzoic acid, 0.69 g (5.6 mmol) of 4-Dimethylaminopyridine are dissolved in 100 ml of dichloromethane. 11.89 g (62.0 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture was partitioned between dichloromethane and water; the organic phase is washed repeatedly with water, dried over sodium sulphate, filtered and concentrated by rotary evaporation. Crystallization form 2-propanol at 0° C. give 17.1 g 4-formylphenyl-4-(4,4,4-trifluorobutoxy)benzoate as colourless crystals.

### 1.3 Preparation of (2E)-3-(4-{[4-(4,4,4-trifluorobutoxy)benzoyl]oxy}phenyl)acrylic acid

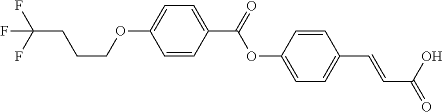

5.00 g (14.2 mMol) of 4-formylphenyl 4-(4,4,4-trifluorobutoxy)benzoate and 3.00 g (28.4 mMol) of Malonic acid are dissolved in 18 ml (227.1 mMol) of Pyridin. 1.21 g (14.2 mMol) of Piperidin are added to the suspension which is allowed to react at 100° C. under argon for 1.5 h. The yellow solution is then thrown on ice. The solution is carefully acidified to pH=1-2 with a 25% HCl solution and is stirred for 15 min. The product is filtrated off and dried at room temperature under vacuum for 10 h to give 5.2 g of (2E)-3-(4-{[4-(4,4,4-trifluorobutoxy)benzoyl]oxy}phenyl)acrylic acid as white powder.

(2E) 3-{4-[(4-(4,4,5,5,5-pentafluoropentyloxy)benzoyl)oxy]phenyl}acrylic acid is prepared analogous to example 1 using 4,4,5,5,5-pentafluoropentan-1-ol instead of 4,4,4-trifluorobutan-1-ol.

The following acrylic acid are synthesized in an analogous manner:
(2E) 3-{4-[(4-trifluoromethoxybenzoyl)oxy]phenyl}acrylic acid
(2E) 3-{4-[(4-(2,2,2-trifluoroethoxy)benzoyl)oxy]phenyl}acrylic acid
(2E) 3-{4-[(4-(2,2,2-trifluoropropoxy)benzoyl)oxy]phenyl}acrylic acid
(2E) 3-{4-[(4-(5,5,5-trifluoropentyloxy)benzoyl)oxy]phenyl}acrylic acid
(2E) 3-{4-[(4-(1,1,2,2-tetrafluoroethoxy)benzoyl)oxy]phenyl}acrylic acid
(2E) 3-{4-[(4-(1,1,2,2-tetrafluoropropoxy)benzoyl)oxy]phenyl}acrylic acid
(2E) 3-{4-[(4-(4,4,5,5,6,6,6-heptafluorohexyloxy)benzoyl)oxy]phenyl}acrylic acid

### EXAMPLE 2

### Synthesis

### Preparation of 6-{[((2E)-3-{4-[(4-(4,4,4-trifluorobutoxy)benzoyl)oxy]phenyl}prop-2-enoyl)oxy]}hexyl 3,5-diaminobenzoate

### 2.1 Preparation of (2E)-3-{4-[(ethoxycarbonyl)oxy]phenyl}acrylic acid

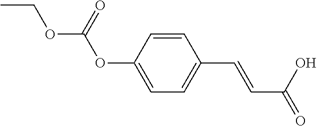

67 g (0.41 mol) p-cumaric acid are added to a mixture of 50.4 g (0.90 mol) potassium hydroxide and 600 ml water. 53.1 g (0.50 mol) ethyl chloroformate are added dropwise at 0° C. The reaction temperature rises to 10° C. The reaction mixture is subsequently allowed to react for 2 hours at 25° C. and acidified to pH=1 with 200 ml hydrochloric acid 7N. The product is filtered off, washed with water and dried under vacuum to give 95.3 g of (2E)-3-{4-[(ethoxycarbonyl)oxy]phenyl}acrylic acid as white powder.

### 2.2 Preparation of 6-hydroxyhexyl 3,5-dinitrobenzoate

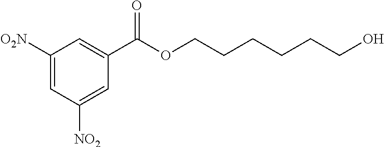

357.70 g (1.686 Mol) of 3,5-dinitrobenzoic acid are suspended in 750 ml of 1-methyl-2-pyrrolidone. The suspension is stirred up to 50° C. 386.36 g (4.599 Mol) of sodium hydrogen carbonate are added and the mixture was heated up to 90° C. 22.50 g (0.150 Mol) of sodium iodide and 204.0 ml (1.533 Mol) of 6-chlorohexanol are added to the reaction mixture which is heated to 100° C. for 1 h. After 1 h of reaction, the reaction is complete and the orange suspension is thrown on 2 l of ice and 1 l of water. The product is filtrated, washed water and dried at 50° C. under vacuum for 24 h to give 425.0 g (91%) of 6-hydroxyhexyl 3,5-dinitrobenzoate as a rose powder.

### 2.3 Preparation of 6-[((2E)-{4-[(ethoxycarbonyl)oxy]phenyl}prop-2-enoyl)oxy]hexyl 3,5-dinitrobenzoate

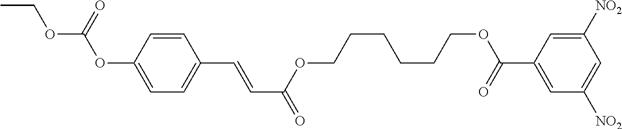

4.53 g (0.0145 Mol) of 6-hydroxyhexyl 3,5-dinitrobenzoate, 3.44 g (0.0145 Mol) of 4-ethylcarbonatecinnamic acid, 0.177 g (0.0015 Mol) of 4-Dimethylaminopyridine are dissolved in 40 ml of dichloromethane. 3.04 g (0.0159 Mol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water; the organic phase is washed repeatedly with water, dried over sodium sulphate, filtered and concentrated by rotary evaporation. The residue is dissolved ethyl acetate. The product is precipitated with Hexane at 0° C. The precipitated is filtrated and dried under vacuum overnight to give 4.2 g (55%) of 6-[((2E)-{4[(ethoxycarbonyl)oxy]phenyl}prop-2-enoyl)oxy]hexyl 3,5-dinitrobenzoate as a light-yellow powder.

### 2.4 Preparation of 6-[((2E)-{4-hydroxyphenyl}prop-2-enoyl)oxy]hexyl 3,5-dinitrobenzoate

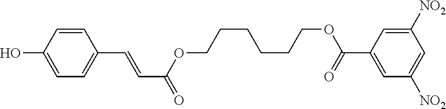

43.20 g (0.081 Mol) of 6-[((2E)-{4[(ethoxycarbonyl)oxy]phenyl}prop-2-enoyl)oxy]hexyl 3,5-dinitrobenzoate are dissolved in 66 ml (0.815 Mol) of pyridine and 400 ml of acetone at room temperature. 61 ml (0.815 Mol) of ammonium hydroxide solution 25% are added dropwise to the solution at room temperature. After 12 h reaction, the mixture is thrown on water and acidified by the addition of HCl 25% (up to pH=3-4). A paste is obtained which is filtrated and dissolved in ethyl acetate and extracted with water. The organic phase is dried with sodium sulfate, filtrated, concentrated by rotary evaporation. Filtration of the residue over silica gel with tert-Butyl methyl ether as eluant and crystallization of the residue in 200 ml of ethyl acetate and 1200 ml of hexane at 0° C. give 15.84 g of 6-[((2E)-{4-hydroxyphenyl}prop-2-enoyl)oxy]hexyl 3,5-dinitrobenzoate as yellow crystals.

### 2.5 Preparation of 6-{[((2E)-3-{4-[(4-(4,4,4-trifluorobutoxy)benzoyl)oxy]phenyl}prop-2-enoyl)oxy]}hexyl 3,5-dinitrobenzoate

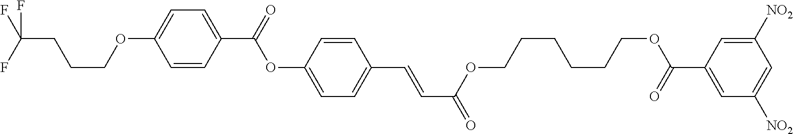

8.61 g (0.0347 Mol) of 4-(4,4,4-trifluorobutoxy)benzoic acid are suspended in 100 ml of dichloromethane. 0.42 g (0.0035 Mol) of 4-Dimethylaminopyridine are added at room temperature. 7.98 g (0.04163 Mol) of N-(3-Dimethylaminopropyl)-N′-ethylcarbodiimide hydrochloride (EDC hydrochloride) are added at 0° C. The solution is stirred for 1 h at 0° C. 15.90 g (0.0347 Mol) of 6-[((2E)-{4-hydroxyphenyl}prop-2-enoyl)oxy]hexyl 3,5-dinitrobenzoate dissolved in 50 ml of dichloromethane are added dropwise to the solution at 0° C. and allowed to stir at room temperature overnight. After 22 hours at room temperature the reaction mixture is partitioned between dichloromethane and water. The mixture is acidified with HCl 25%. The organic phase is washed repeatedly with water, dried over sodium sulphate, filtered and concentrated by rotary evaporation. Chromatography of the residue on 600 g silica gel using toluene:ethyl acetate (99:1) as eluant and crystallization from ethyl acetate/hexane (1:2) yielded 18.82 g (79%) of 6-{[((2E)-3-{4-[(4-(4,4,4-trifluorobutoxy)benzoyl)oxy]phenyl}prop-2-enoyl)oxy]}hexyl 3,5-dinitrobenzoate as white crystals.

### 2.6 Preparation of 6-{[((2E)-3-{4-[(4-(4,4,4-trifluorobutoxy)benzoyl)oxy]phenyl}prop-2-enoyl)oxy]}hexyl 3,5-diaminobenzoate

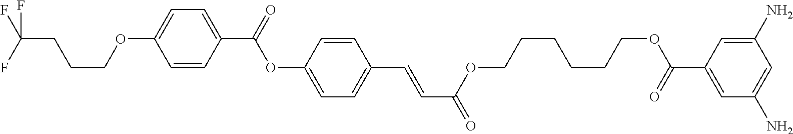

18.80 g (0.027 Mol) of 6-{[((2E)-3-{4-[(4-(4,4,4-trifluorobutoxy)benzoyl)oxy]phenyl}prop-2-enoyl)oxy]}hexyl 3,5-dinitrobenzoate are dissolved in a mixture of 350 ml of N,N-dimethylformamide and 25 ml water. 44.28 g (0.164 Mol) ferric chloride hexahydrate are added. 17.85 g (0.273 Mol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 400 g silica gel using toluene:ethyl acetate (2:1) as eluant yielded 15.39 g (91%) of 6-{[((2E)-3-{4-[(4-(4,4,4-trifluorobutoxy)benzoyl)oxy]phenyl}prop-2-enoyl)oxy]}hexyl 3,5-diaminobenzoate as yellowish crystals.

The following diamines are synthesized in an analogous manner:
2-{[((2E)-3-{4-[(4-(trifluoroethoxy)benzoyl)oxy]phenyl}prop-2-enoyl)oxy]}ethyl 3,5-diaminobenzoate.
3-{[((2E)-3-{4-[(4-(trifluoroethoxy)benzoyl)oxy]phenyl}prop-2-enoyl)oxy]}propyl 3,5-diaminobenzoate.
4-{[((2E)-3-{4-[(4-(trifluoroethoxy)benzoyl)oxy]phenyl}prop-2-enoyl)oxy]}butyl 3,5-diaminobenzoate.
5-{[((2E)-3-{4-[(4-(trifluoroethoxy)benzoyl)oxy]phenyl}prop-2-enoyl)oxy]}pentyl 3,5-diaminobenzoate
7-{[((2E)-3-{4-[(4-(trifluoroethoxy)benzoyl)oxy]phenyl}prop-2-enoyl)oxy]}heptyl 3,5-diaminobenzoate.
8-{[((2E)-3-{4-[(4-(trifluoroethoxy)benzoyl)oxy]phenyl}prop-2-enoyl)oxy]}octyl 3,5-diaminobenzoate.
11-{[((2E)-3-{4-[(4-(trifluoroethoxy)benzoyl)oxy]phenyl}prop-2-enoyl)oxy]}undecyl 3,5-diaminobenzoate.
2-{[((2E)-3-{4-[(4-(trifluoromethoxy)benzoyl)oxy]phenyl}prop-2-enoyl)oxy]}ethyl 3,5-diaminobenzoate.
3-{[((2E)-3-{4-[(4-(trifluoromethoxy)benzoyl)oxy]phenyl}prop-2-enoyl)oxy]}propyl 3,5-diaminobenzoate.
4-{[((2E)-3-{4-[(4-(trifluoromethoxy)benzoyl)oxy]phenyl}prop-2-enoyl)oxy]}butyl 3,5-diaminobenzoate.
5-{[((2E)-3-{4-[(4-(trifluoromethoxy)benzoyl)oxy]phenyl}prop-2-enoyl)oxy]}pentyl 3,5-diaminobenzoate
6-{[((2E)-3-{4-[(4-(trifluoromethoxy)benzoyl)oxy]phenyl}prop-2-enoyl)oxy]}hexyl 3,5-diaminobenzoate
7-{[((2E)-3-{4-[(4-(trifluoromethoxy)benzoyl)oxy]phenyl}prop-2-enoyl)oxy]}heptyl 3,5-diaminobenzoate.
8-{[((2E)-3-{4-[(4-(trifluoromethoxy)benzoyl)oxy]phenyl}prop-2-enoyl)oxy]}octyl 3,5-diaminobenzoate.
2-{[(((2E)-3-{4-[(4-(trifluoromethyl)benzoyl)oxy]phenyl}prop-2-enoyl)oxy]}ethyl 3,5-diaminobenzoate.
3-{[((2E)-3-{4-[(4-(trifluoromethyl)benzoyl)oxy]phenyl}prop-2-enoyl)oxy]}propyl 3,5-diaminobenzoate.
4-{[((2E)-3-{4-[(4-(trifluoromethyl)benzoyl)oxy]phenyl}prop-2-enoyl)oxy]}butyl 3,5-diaminobenzoate.
5-{[((2E)-3-{4-[(4-(trifluoromethyl)benzoyl)oxy]phenyl}prop-2-enoyl)oxy]}pentyl 3,5-diaminobenzoate
6-{[((2E)-3-{4-[(4-(trifluoromethyl)benzoyl)oxy]phenyl}prop-2-enoyl)oxy]}hexyll 3,5-diaminobenzoate
8-{[((2E)-3-{4-[(4-(trifluoromethyl)benzoyl)oxy]phenyl}prop-2-enoyl)oxy]}octyl 3,5-diaminobenzoate
11-{[((2E)-3-{4-[(4-(trifluoromethyl)benzoyl)oxy]phenyl}prop-2-enoyl)oxy]}undecyll 3,5-diaminobenzoate
2-[2-{[((2E)-3-{4-[(4-(trifluoroethoxy)benzoyl)oxy]phenyl}prop-2-enoyl)oxy]}ethoxy]ethyl 3,5-diaminobenzoate
2{2-[2-{[((2E)-3-{4-[(4-(trifluoroethoxy)benzoyl)oxy]phenyl}prop-2-enoyl)oxy]}ethoxy]ethoxy}ethyl 3,5-diaminobenzoate
2,2-dimethyl-3-{[((2E)-3-{4-[(4-(trifluoroethoxy)benzoyl)oxy]phenyl}prop-2-enoyl)oxy]}propyl 3,5-diaminobenzoate
2-{[((2E)-3-{4-[(4-(3,3,3-trifluoropropoxy)benzoyl)oxy]phenyl}prop-2-enoyl)oxy]}ethyl 3,5-diaminobenzoate
3-{[((2E)-3-{4-[(4-(3,3,3-trifluoropropoxy)benzoyl)oxy]phenyl}prop-2-enoyl)oxy]}propyl 3,5-diaminobenzoate
4-{[((2E)-3-{4-[(4-(3,3,3-trifluoropropoxy)benzoyl)oxy]phenyl}prop-2-enoyl)oxy]}butyl 3,5-diaminobenzoate.
6-{[((2E)-3-{4-[(4-(3,3,3-trifluoropropoxy)benzoyl)oxy]phenyl}prop-2-enoyl)oxy]}hexyl 3,5-diaminobenzoate
7-{[((2E)-3-{4-[(4-(3,3,3-trifluoropropoxy)benzoyl)oxy]phenyl}prop-2-enoyl)oxy]}heptyl 3,5-diaminobenzoate
8-{[((2E)-3-{4-[(4-(3,3,3-trifluoropropoxy)benzoyl)oxy]phenyl}prop-2-enoyl)oxy]}octyl 3,5-diaminobenzoate
11-{[((2E)-3-{4-[(4-(3,3,3-trifluoropropoxy)benzoyl)oxy]phenyl}prop-2-enoyl)oxy]}undecyl 3,5-diaminobenzoate
2-[2-{[((2E)-3-{4-[(4-(3,3,3-trifluoropropoxy)benzoyl)oxy]phenyl}prop-2-enoyl)oxy]}ethoxy]ethyl 3,5-diaminobenzoate
2{2-[2-{[((2E)-3-{4-[(4-(3,3,3-trifluoropropoxy)benzoyl)oxy]phenyl}prop-2-enoyl)oxy]}ethoxy]ethoxy}ethyl 3,5-diaminobenzoate
2,2-dimethyl-3-{[((2E)-3-{4-[(4-(3,3,3-trifluoropropoxy)benzoyl)oxy]phenyl}prop-2-enoyl)oxy]}propyl 3,5-diaminobenzoate
2-{[((2E)-3-{4-[(4-(4,4,4-trifluorobutoxy)benzoyl)oxy]phenyl}prop-2-enoyl)oxy]}ethyl 3,5-diaminobenzoate
3-{[((2E)-3-{4-[(4-(4,4,4-trifluorobutoxy)benzoyl)oxy]phenyl}prop-2-enoyl)oxy]}propyl 3,5-diaminobenzoate.
4-{[((2E)-3-{4-[(4-(4,4,4-trifluorobutoxy)benzoyl)oxy]phenyl}prop-2-enoyl)oxy]}butyl 3,5-diaminobenzoate.
5-{[((2E)-3-{4-[(4-(4,4,4-trifluorobutoxy)benzoyl)oxy]phenyl}prop-2-enoyl)oxy]}pentyl 3,5-diaminobenzoate
7-{[((2E)-3-{4-[(4-(4,4,4-trifluorobutoxy)benzoyl)oxy]phenyl}prop-2-enoyl)oxy]}heptyl 3,5-diaminobenzoate.
8-{[((2E)-3-{4-[(4-(4,4,4-trifluorobutoxy)benzoyl)oxy]phenyl}prop-2-enoyl)oxy]}octyl 3,5-diaminobenzoate.
11-{[((2E)-3-{4-[(4-(4,4,4-trifluorobutoxy)benzoyl)oxy]phenyl}prop-2-enoyl)oxy]}undecyl 3,5-diaminobenzoate.
2-[2-{[((2E)-3-{4-[(4-(4,4,4-trifluorobutoxy)benzoyl)oxy]phenyl}prop-2-enoyl)oxy]}ethoxy]ethyl 3,5-diaminobenzoate
2{2-[2-{[((2E)-3-{4-[(4-(4,4,4-trifluorobutoxy)benzoyl)oxy]phenyl}prop-2-enoyl)oxy]}ethoxy]ethoxy}ethyl 3,5-diaminobenzoate
2,2-dimethyl-3-{[((2E)-3-{4-[(4-(4,4,4-trifluorobutoxy)benzoyl)oxy]phenyl}prop-2-enoyl)oxy]}propyl 3,5-diaminobenzoate
2-{[((2E)-3-{4-[(4-(5,5,5-trifluoropentoxy)benzoyl)oxy]phenyl}prop-2-enoyl)oxy]}ethyl 3,5-diaminobenzoate
3-{[((2E)-3-{4-[(4-(5,5,5-trifluoropentoxy)benzoyl)oxy]phenyl}prop-2-enoyl)oxy]}propyl 3,5-diaminobenzoate
4-{[((2E)-3-{4-[(4-(5,5,5-trifluoropentoxy)benzoyl)oxy]phenyl}prop-2-enoyl)oxy]}butyl 3,5-diaminobenzoate
5-{[((2E)-3-{4-[(4-(5,5,5-trifluoropentoxy)benzoyl)oxy]phenyl}prop-2-enoyl)oxy]}pentyl 3,5-diaminobenzoate
6-{[((2E)-3-{4-[(4-(5,5,5-trifluoropentoxy)benzoyl)oxy]phenyl}prop-2-enoyl)oxy]}hexyl 3,5-diaminobenzoate
8-{[((2E)-3-{4-[(4-(5,5,5-trifluoropentoxy)benzoyl)oxy]phenyl}prop-2-enoyl)oxy]}octyl 3,5-diaminobenzoate
11-{[((2E)-3-{4-[(4-(5,5,5-trifluoropentoxy)benzoyl)oxy]phenyl}prop-2-enoyl)oxy]}undecyl 3,5-diaminobenzoate
2-[2-{[((2E)-3-{4-[(4-(5,5,5-trifluoropentoxy)benzoyl)oxy]phenyl}prop-2-enoyl)oxy]}ethoxy]ethyl 3,5-diaminobenzoate
2{2-[2-{[((2E)-3-{4-[(4-(5,5,5-trifluoropentoxy)benzoyl)oxy]phenyl}prop-2-enoyl)oxy]}ethoxy]ethoxy}ethyl 3,5-diaminobenzoate
2,2-dimethyl-3-{[((2E)-3-{4-[(4-(5,5,5-trifluoropentoxy)benzoyl)oxy]phenyl}prop-2-enoyl)oxy]}propyl 3,5-diaminobenzoate
2-{[((2E)-3-{4-[(4-(6,6,6-trifluorohexyloxy)benzoyl)oxy]phenyl}prop-2-enoyl)oxy]}ethyl 3,5-diaminobenzoate
3-{[((2E)-3-{4-[(4-(6,6,6-trifluorohexyloxy)benzoyl)oxy]phenyl}prop-2-enoyl)oxy]}propyl 3,5-diaminobenzoate
4-{[((2E)-3-{4-[(4-(6,6,6-trifluorohexyloxy)benzoyl)oxy]phenyl}prop-2-enoyl)oxy]}butyl 3,5-diaminobenzoate
5-{[((2E)-3-{4-[(4-(6,6,6-trifluorohexyloxy)benzoyl)oxy]phenyl}prop-2-enoyl)oxy]}pentyl 3,5-diaminobenzoate
7-{[((2E)-3-{4-[(4-(6,6,6-trifluorohexyloxy)benzoyl)oxy]phenyl}prop-2-enoyl)oxy]}heptyl 3,5-diaminobenzoate
8-{[((2E)-3-{4-[(4-(6,6,6-trifluorohexyloxy)benzoyl)oxy]phenyl}prop-2-enoyl)oxy]}octyl 3,5-diaminobenzoate
11-{[((2E)-3-{4-[(4-(6,6,6-trifluorohexyloxy)benzoyl)oxy]phenyl}prop-2-enoyl)oxy]}undecyl 3,5-diaminobenzoate
2-[2-{[((2E)-3-{4-[(4-(6,6,6-trifluorohexyloxy)benzoyl)oxy]phenyl}prop-2-enoyl)oxy]}ethoxy]ethyl 3,5-diaminobenzoate
2{2-[2-{[((2E)-3-{4-[(4-(6,6,6-trifluorohexyloxy)benzoyl)oxy]phenyl}prop-2-enoyl)oxy]}ethoxy]ethoxy}ethyl 3,5-diaminobenzoate
2,2-dimethyl-3-{[((2E)-3-{4-[(4-(6,6,6-trifluorohexyloxy)benzoyl)oxy]phenyl}prop-2-enoyl)oxy]}propyl 3,5-diaminobenzoate
3-{[((2E)-3-{4-[(3-methoxy 4-(3,3,3-trifluoropropoxy)benzoyl)oxy]phenyl}prop-2-enoyl)oxy]}propyl 3,5-diaminobenzoate
8-{[((2E)-3-{4-[(3-methoxy 4-(3,3,3-trifluoropropoxy)benzoyl)oxy]phenyl}prop-2-enoyl)oxy]}octyl 3,5-diaminobenzoate
11-{[((2E)-3-{4-[(3-methoxy 4-(3,3,3-trifluoropropoxy)benzoyl)oxy]phenyl}prop-2-enoyl)oxy]}undecyl 3,5-diaminobenzoate
6-{[((2E)-3-{4-[(3-methoxy 4-(4,4,4-trifluorobutoxy)benzoyl)oxy]phenyl}prop-2-enoyl)oxy]}hexyll 3,5-diaminobenzoate
8-{[((2E)-3-{4-[(3-methoxy 4-(4,4,4-trifluorobutoxy)benzoyl)oxy]phenyl}prop-2-enoyl)oxy]}octyl 3,5-diaminobenzoate
11-{[((2E)-3-{4-[(3-methoxy 4-(4,4,4-trifluorobutoxy)benzoyl)oxy]phenyl}prop-2-enoyl)oxy]}undecyl 3,5-diaminobenzoate
4-{[((2E)-3-{4-[(3-methoxy 4-(5,5,5-trifluoropentoxy)benzoyl)oxy]phenyl}prop-2-enoyl)oxy]}butyl 3,5-diaminobenzoate
6-{[((2E)-3-{4-[(3-methoxy 4-(5,5,5-trifluoropentoxy)benzoyl)oxy]phenyl}prop-2-enoyl)oxy]}hexyl3,5-diaminobenzoate
4-{[((2E)-3-{4-[(3-methoxy 4-(6,6,6-trifluorohexyloxy)benzoyl)oxy]phenyl}prop-2-enoyl)oxy]}butyl 3,5-diaminobenzoate
6-{[((2E)-3-{4-[(3-methoxy 4-(6,6,6-trifluorohexyloxy)benzoyl)oxy]phenyl}prop-2-enoyl)oxy]}hexyl 3,5-diaminobenzoate
2-{[((2E)-3-{4-[(4-(4, 4, 5, 5,5-pentafluoropentoxy)benzoyl)oxy]phenyl}prop-2-enoyl)oxy]}ethyl 3,5-diaminobenzoate
3-{[((2E)-3-{4-[(4-(4,4,5,5,5-pentafluoropentoxy)benzoyl)oxy]phenyl}prop-2-enoyl)oxy]}propyl 3,5-diaminobenzoate
4-{[((2E)-3-{4-[(4-(4,4,5,5,5-pentafluoropentoxy)benzoyl)oxy]phenyl}prop-2-enoyl)oxy]}butyl 3,5-diaminobenzoate
6-{[((2E)-3-{4-[(4-(4,4,5,5,5-pentafluoropentoxy)benzoyl)oxy]phenyl}prop-2-enoyl)oxy]}hexyl 3,5-diaminobenzoate
7-{[((2E)-3-{4-[(4-(4,4,5,5,5-pentafluoropentoxy)benzoyl)oxy]phenyl}prop-2-enoyl)oxy]}heptyl 3,5-diaminobenzoate
8-{[((2E)-3-{4-[(4-(4,4,5,5,5-pentafluoropentoxy)benzoyl)oxy]phenyl}prop-2-enoyl)oxy]}octyl 3,5-diaminobenzoate
11-{[((2E)-3-{4-[(4-(4,4,5,5,5-pentafluoropentoxy)benzoyl)oxy]phenyl}prop-2-enoyl)oxy]}undecyl 3,5-diaminobenzoate
2-[2-{[((2E)-3-{4-[(4-(4,4,5,5,5-pentafluoropentoxy)benzoyl)oxy]phenyl}prop-2-enoyl)oxy]}ethoxy]ethyl 3,5-diaminobenzoate
2{2-[2-{[((2E)-3-{4-[(4-(4,4,5,5,5-pentafluoropentoxy)benzoyl)oxy]phenyl}prop-2-enoyl)oxy]}ethoxy]ethoxy}ethyl 3,5-diaminobenzoate
2,2-Dimethyl-3-{[((2E)-3-{4-[(4-(4,4,5,5,5-pentafluoropentoxy)benzoyl)oxy]phenyl}prop-2-enoyl)oxy]}propyl 3,5-diaminobenzoate
2-{[((2E)-3-{4-[(4-(1,1,2,2-tetrafluoroethoxy)benzoyl)oxy]phenyl}prop-2-enoyl)oxy]}ethyl 3,5-diaminobenzoate
3-{[((2E)-3-{4-[(4-(1,1,2,2-tetrafluoroethoxy)benzoyl)oxy]phenyl}prop-2-enoyl)oxy]}propyl 3,5-diaminobenzoate
4-{[((2E)-3-{4-[(4-(1,1,2,2-tetrafluoroethoxy)benzoyl)oxy]phenyl}prop-2-enoyl)oxy]}butyl 3,5-diaminobenzoate
5-{[((2E)-3-{4-[(4-(1,1,2,2-tetrafluoroethoxy)benzoyl)oxy]phenyl}prop-2-enoyl)oxy]}pentyl 3,5-diaminobenzoate
6-{[((2E)-3-{4-[(4-(1,1,2,2-tetrafluoroethoxy)benzoyl)oxy]phenyl}prop-2-enoyl)oxy]}hexyll 3,5-diaminobenzoate
7-{[((2E)-3-{4-[(4-(1,1,2,2-tetrafluoroethoxy)benzoyl)oxy]phenyl}prop-2-enoyl)oxy]}heptyl 3,5-diaminobenzoate.
8-{[((2E)-3-{4-[(4-(1,1,2,2-tetrafluoroethoxy)benzoyl)oxy]phenyl}prop-2-enoyl)oxy]}octyl 3,5-diaminobenzoate
11-{[((2E)-3-{4-[(4-(1,1,2,2-tetrafluoroethoxy)benzoyl)oxy]phenyl}prop-2-enoyl)oxy]}undecyl 3,5-diaminobenzoate
6-{[((2E)-3-{4-[(4-{[(4,4,4-trifluorobutoxy)carbonyl]amino}benzoyl)oxy]phenyl}prop-2-enoyl)oxy]}hexyll 3,5-diaminobenzoate
6-{[((2E)-3-{4-[(4-{[(4,4,5,5,5-pentafluoropentoxy)carbonyl]amino}benzoyl)oxy]phenyl}prop-2-enoyl)oxy]}hexyll 3,5-diaminobenzoate
6-{[((2E)-3-{4-[(4-({[(4,4,5,5,6,6,6-heptafluorohexyloxy)carbonyl]amino})benzoyl)oxy]phenyl}prop-2-enoyl)oxy]}hexyll 3,5-diaminobenzoate

### EXAMPLE 3

### Synthesis

### Preparation of 3,5-Diaminobenzyl (2E) 3-{4-[(4-(4,4,4-trifluorobutoxy)benzoyl)oxy]phenyl}acrylate

### 3.1 Preparation of 3,5-dinitrobenzyl (2E)3-{4-[(4-(4,4,4-trifluorobutoxy)benzoyl)oxy]phenyl}acrylate

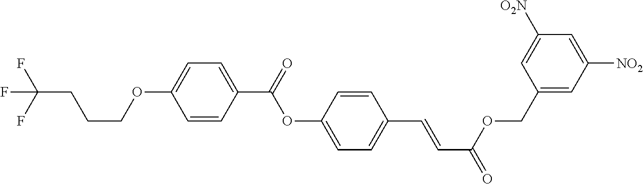

1.00 g (51.0 mmol) of 3,5-dinitrobenzylalcohol, 2.00 g (51.0 mmol) of (2E)-3-(4-{[4-(4,4,4-trifluorobutoxy)benzoyl]oxy}phenyl)acrylic acid, 62 mg (0.51 mmol) of 4-Dimethylaminopyridine are dissolved in 10 ml of dichloromethane. 1.07 g (56.0 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation to yield 3,5-dinitrobenzyl (2E)3-{4-[(4-(4,4,4-trifluorobutoxy)benzoyl)oxy]phenyl}acrylate 2.1 g as colorless crystals.

### 3.2 Preparation of 3,5-Diaminobenzyl (2E) 3-{4-[(4-(4,4,4-trifluorobutoxy)benzoyl)oxy]phenyl}acrylate

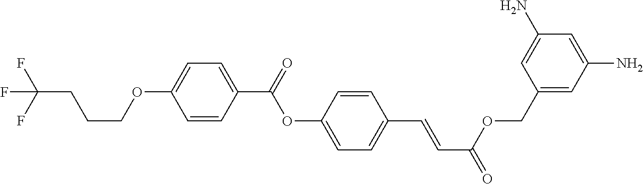

5.30 g (9.22 mmol) of (2E)3-{4-[(4-(4,4,4-trifluorobutoxy)benzoyl)oxy]phenyl}acrylate are dissolved in a mixture of 55 ml of N,N-dimethylformamide and 6 ml water. 14.98 g (55.3 mmol) ferric chloride hexahydrate are added. 6.03 g (91.8 mmol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 200 g silica gel using toluene:ethyl acetate (1:1) as eluant and crystallization form ethylacetate:hexane mixture yielded 3.8 g 3,5-Diaminobenzyl (2E) 3-{4-[(4-(4,4,4-trifluorobutoxy)benzoyl)oxy]phenyl}acrylate as yellowish crystals

The following diamines are synthesized in an analogous manner:
3,5-Diaminobenzyl (2E) 3-{4-[(4-trifluoromethoxybenzoyl)oxy]phenyl}acrylate
3,5-Diaminobenzyl (2E) 3-{4-[(4-(2,2,2-trifluoroethoxy)benzoyl)oxy]phenyl}acrylate
3,5-Diaminobenzyl (2E) 3-{4-[(4-(5,5,5-trifluoropentyloxy)benzoyl)oxy]phenyl}acrylate
3,5-Diaminobenzyl (2E) 3-{4-[(4-(4,4,5,5,5-pentafluoropentyloxy)benzoyl)oxy]phenyl}acrylate
3,5-Diaminobenzyl (2E) 3-{4-[(4-(3,3,4,4,5,5,6,6,6-nonafluorohexyloxy)benzoyl)oxy]phenyl}acrylate
3,5-Diaminobenzyl (2E) 3-{4-[(4-(2,2,3,3,3-pentafluoropropyloxy)benzoyl)oxy]phenyl}acrylate
3,5-Diaminobenzyl (2E) 3-{4-[(4-(2,2,3,4,4,4-hexafluorobutoxy)benzoyl)oxy]phenyl}acrylate
3,5-Diaminobenzyl (2E) 3-{4-[(4-(1,1,2,2-tetrafluoroethoxy)benzoyl)oxy]phenyl}acrylate
3,5-Diaminobenzyl (2E) 3-{4-[(4-(1,1,2,2-tetrafluoropropoxy)benzoyl)oxy]phenyl}acrylate
3,5-Diaminobenzyl (2E) 3-{4-[(4-(4,4,5,5,6,6,6-heptafluorohexyloxy)benzoyl)oxy]phenyl}acrylate
3,5-Diaminobenzyl (2E) 3-{4-[(4-(4,5,5,5-tetrafluoro-4-(trifluoromethyl)pentyloxy)benzoyl)oxy]phenyl}acrylate
3,5-Diaminobenzyl (2E) 3-{4-{[(4-(4,4,5,5,6,6,6-heptafluorohexyloyl)oxy)benzoyl oxy]phenyl}acrylate
3,5-Diaminobenzyl (2E) 3-{4-[(4-[(4,4,4-trifluorobutoxy)carbonyl]amino)benzoyl)oxy]phenyl}acrylate
3,5-Diaminobenzyl (2E) 3-{4-[(4-[(4,4,4-trifluoropentyloxy)carbonyl]amino) benzoyl]oxy}phenyl}acrylate
3,5-Diaminobenzyl (2E) 3-{4-[4-(4,4,5,5,5,-pentafluoropentyloyloxy)benzoyl oxy]phenyl}acrylate
3,5-Diaminobenzyl (2E) 3-{4-[4-(4,4,5,5,6,6,6-heptafluorohexyloyloxy)benzoyl oxy]phenyl}acrylate
3,5-Diaminobenzyl (2E) 3-{4-[(3-fluoro-4-(4,4,4-trifluorobutoxy)benzoyl)oxy]phenyl}acrylate
2,5-Diaminobenzyl (2E) 3-{4-[(4-trifluoromethoxybenzoyl)oxy]phenyl}acrylate
2,5-Diaminobenzyl (2E) 3-{4-[(4-(2,2,2-trifluoroethoxy)benzoyl)oxy]phenyl}acrylate
2,5-Diaminobenzyl (2E) 3-{4-[(4-(5,5,5-trifluorobutoxy)benzoyl)oxy]phenyl}acrylate
2,5-Diaminobenzyl (2E) 3-{4-[(4-(5,5,5-trifluoropentyloxy)benzoyl)oxy]phenyl}acrylate
2,5-Diaminobenzyl (2E) 3-{4-[(4-(4,4,5,5,5-pentafluoropentyloxy)benzoyl)oxy]phenyl}acrylate
2,5-Diaminobenzyl (2E) 3-{4-[(4-(1,1,2,2-tetrafluoroethoxy)benzoyl)oxy]phenyl}acrylate
2,5-Diaminobenzyl (2E) 3-{4-[(4-(1,1,2,2-tetrafluoropropoxy)benzoyl)oxy]phenyl}acrylate
2,5-Diaminobenzyl (2E) 3-{4-[(4-(4,4,5,5,6,6,6-heptafluorohexyloxy)benzoyl)oxy]phenyl}acrylate
2,5-Diaminobenzyl (2E) 3-{4-{[(4-(4,4,5,5,6,6,6-heptafluorohexyloyl)oxy)benzoyl oxy]phenyl}acrylate
2,5-Diaminobenzyl (2E) 3-{4-[(4-[(4,4,4-trifluorobutoxy)carbonyl]amino)benzoyl)oxy]phenyl}acrylate
2,5-Diaminobenzyl (2E) 3-{4-[(4-[(4,4,4-trifluoropentyloxy)carbonyl]amino) benzoyl]oxy}phenyl}acrylate
2,5-Diaminobenzyl (2E) 3-{4-[4-(4,4,5,5,5,-pentafluoropentyloyloxy)benzoyl oxy]phenyl}acrylate
2,5-Diaminobenzyl (2E) 3-{4-[4-(4,4,5,5,6,6,6-heptafluorohexyloyloxy)benzoyl oxy]phenyl}acrylate
2,5-Diaminobenzyl (2E) 3-{4-[(2-fluoro-4-(4,4,4-trifluorobutoxy)benzoyl)oxy]phenyl}acrylate
2,4-Diaminobenzyl (2E) 3-{4-[(4-trifluoromethoxybenzoyl)oxy]phenyl}acrylate
2,4-Diaminobenzyl (2E) 3-{4-[(4-(2,2,2-trifluoroethoxy)benzoyl)oxy]phenyl}acrylate
2,4-Diaminobenzyl (2E) 3-{4-[(4-(5,5,5-trifluoropentyloxy)benzoyl)oxy]phenyl}acrylate
2,4-Diaminobenzyl (2E) 3-{4-[(4-(4,4,4-trifluorobutoxy)benzoyl)oxy]phenyl}acrylate
2,4-Diaminobenzyl (2E) 3-{4-[(4-(4,4,5,5,5-pentafluoropentyloxy)benzoyl)oxy]phenyl}acrylate
2,4-Diaminobenzyl (2E) 3-{4-[(4-(1,1,2,2-tetrafluoroethoxy)benzoyl)oxy]phenyl}acrylate
2,4-Diaminobenzyl (2E) 3-{4-[(4-(1,1,2,2-tetrafluoropropoxy)benzoyl)oxy]phenyl}acrylate
2,4-Diaminobenzyl (2E) 3-{4-[(4-(4,4,5,5,6,6,6-heptafluorohexyloxy)benzoyl)oxy]phenyl}acrylate
2,4-Diaminobenzyl (2E) 3-{4-{[(4-(4,4,5,5,6,6,6-heptafluorohexyloyl)oxy)benzoyl oxy]phenyl}acrylate
2,4-Diaminobenzyl (2E) 3-{4-[(4-[(4,4,4-trifluorobutoxy)carbonyl]amino)benzoyl)oxy]phenyl}acrylate
2,4-Diaminobenzyl (2E) 3-{4-[(4-[(4,4,4-trifluoropentyloxy)carbonyl]amino) benzoyl]oxy}phenyl}acrylate
2,4-Diaminobenzyl (2E) 3-{4-[4-(4,4,5,5,5,-pentafluoropentyloyloxy)benzoyl oxy]phenyl}acrylate
2,4-Diaminobenzyl (2E) 3-{4-[4-(4,4,5,5,6,6,6-heptafluorohexyloyloxy)benzoyl oxy]phenyl}acrylate
2,4-Diaminobenzyl (2E) 3-{4-[(3-fluoro-4-(4,4,4-trifluorobutoxy)benzoyl)oxy]phenyl}acrylate

### EXAMPLE 4

### Synthesis

### Preparation of 2-(2,4-Diaminophenyl)ethyl (2E) 3-{4-[(4-(4,4,5,5,5-pentafluoropentoxy)benzoyl)oxy]phenyl}acrylate

### 4.1 Preparation of 2-(2,4-dinitrophenyl)ethanol

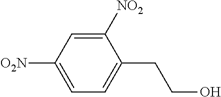

22.6 g (100 mmol) 2,4-dinitrophenylacetic acid are dissolved in 150 ml tetrahydrofuran and added dropwise in a the course of 2 hours to 300 ml (300 mmol) of a borane-tetrahydrofuran complex 1.0 M solution in tetrahydrofuran. After 3 hours at 25° C., 200 ml water are carefully added. The reaction mixture is then partitioned between ethyl acetate and water; the organic phase was washed repeatedly with water, dried over sodium sulfate, filtered and concentrated by rotary evaporation. Chromatography of the residue on 400 g silica gel using toluene:ethyl acetate 1:1 as eluant and crystallization form ethylacetate:hexane mixture to yield 20.7 g (98%) of 2-(2,4-dinitrophenyl)ethanol as yellowish crystals.

### 4.2 Preparation of 2-(2,4-Dinitrophenyl)ethyl (2E) 3-{4-[(4-(4,4,5,5,5-pentafluoropentoxy)benzoyl)oxy]phenyl}acrylate

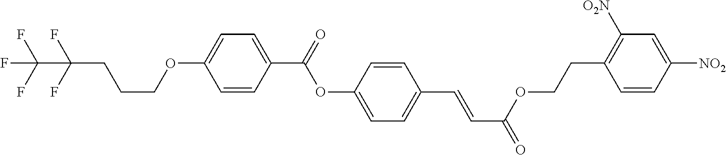

2.50 g (11.8 mmol) of 2-(2,4-dinitrophenyl)ethanol, 5.24 g (11.8 mmol) of (2E)-3-(4-{[4-(4,4,5,5,5-pentafluoropentoxy)benzoyl]oxy}phenyl)acrylic acid, 144 mg (1.2 mmol) of 4-Dimethylaminopyridine are dissolved in 30 ml of dichloromethane. 2.48 g (13.0 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation. Chromatography of the residue on 200 g silica gel using toluene:ethyl acetate 95:5 as eluant and crystallization form ethylacetate:hexane mixture to yield 5.35 g (71%) 2-(2,4-Dinitrophenyl)ethyl (2E) 3-{4-[(4-(4,4,5,5,5-pentafluoropentoxy)benzoyl)oxy]phenyl}acrylate as colorless crystals.

### 4.3 Preparation of 2-(2,4-Diaminophenyl)ethyl (2E) 3-{4-[(4-(4,4,5,5,5-pentafluoropentoxy)benzoyl)oxy]phenyl}acrylate

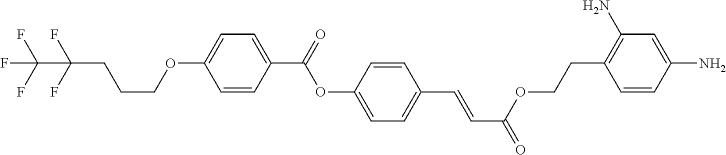

5.35 g (8.38 mmol) of (2E)3-{4-[(4-(4,4,5,5,5-pentafluoropentoxy)benzoyl)oxy]phenyl}acrylate are dissolved in a mixture of 54 ml of N,N-dimethylformamide and 6 ml water. 13.9 g (51.4 mmol) ferric chloride hexahydrate are added. 5.60 g (85.7 mmol) Zinc powder are added portionwise within 60 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Filtration of the residue on 200 g silica gel using toluene:ethyl acetate (1:3) as eluant and crystallization form ethylacetate:hexane mixture yielded 3.30 g 2-(2,4-Diaminophenyl)ethyl (2E) 3-{4-[(4-(4,4,5,5,5-pentafluoropentoxy)benzoyl)oxy]phenyl}acrylate as yellowish crystals 2-(2,4-Diaminophenyl)ethyl (2E) 3-{4-[(4-(4,4,4-trifluorobutoxy)benzoyl)oxy]phenyl}acrylate is prepared analogous to example 4 using (2E)-3-(4-{[(4-(4,4,4-trifluorobutoxy)benzoyl]oxy}phenyl)acrylic acid.

The following diamines are synthesized in an analogous manner:
2-(3,5-Diaminophenyl)ethyl (2E) 3-{4-[(4-trifluoromethoxybenzoyl)oxy]phenyl}acrylate
2-(3,5-Diaminophenyl)ethyl (2E) 3-{4-[(4-(2,2,2-trifluoroethoxy)benzoyl)oxy]phenyl}acrylate
2-(3,5-Diaminophenyl)ethyl (2E) 3-{4-[(4-(4,4,4-trifluorobutoxy)benzoyl)oxy]phenyl}acrylate
2-(3,5-Diaminophenyl)ethyl (2E) 3-{4-[(4-(5,5,5-trifluoropentyloxy)benzoyl)oxy]phenyl}acrylate
2-(3,5-Diaminophenyl)ethyl (2E) 3-{4-[(4-(4,4,5,5,5-pentafluoropentoxy)benzoyl)oxy]phenyl}acrylate
2-(3,5-Diaminophenyl)ethyl (2E) 3-{4-[(4-(1,1,2,2-tetrafluoroethoxy)benzoyl)oxy]phenyl}acrylate
2-(3,5-Diaminophenyl)ethyl (2E) 3-{4-[(4-(4,4,4-trifluorobutanoyl)oxy)benzoyl]oxy}phenyl}acrylate
2-(3,5-Diaminophenyl)ethyl (2E) 3-{4-[(3-methoxy-4-trifluoromethoxybenzoyl)oxy]phenyl}acrylate
2-(3,5-Diaminophenyl)ethyl (2E) 3-{4-[(3-methoxy 4-(2,2,2-trifluoroethoxy)benzoyl)oxy]phenyl}acrylate
2-(3,5-Diaminophenyl)ethyl (2E) 3-{4-[(3-methoxy 4-(4,4,4-trifluorobutoxy)benzoyl)oxy]phenyl}acrylate
2-(3,5-Diaminophenyl)ethyl (2E) 3-{4-[(3-methoxy 4-(5,5,5-trifluoropentyloxy)benzoyl)oxy]phenyl}acrylate
2-(3,5-Diaminophenyl)ethyl (2E) 3-{4-[(3-methoxy 4-(4,4,5,5,5-pentafluoropentoxy)benzoyl)oxy]phenyl}acrylate
2-(3,5-Diaminophenyl)ethyl (2E) 3-{4-[(3-methoxy 4-(1,1,2,2-tetrafluoroethoxy)benzoyl)oxy]phenyl}acrylate
2-(3,5-Diaminophenyl)ethyl (2E) 3-{4-[(3-methoxy 4-(4,4,4-trifluorobutanoyl)oxy)benzoyl]oxy}phenyl}acrylate
2-(3,5-Diaminophenyl)ethyl (2E) 3-{4-[(4-trifluoromethoxybenzoyl)oxy]-3-methoxyphenyl}acrylate
2-(3,5-Diaminophenyl)ethyl (2E) 3-{4-[(4-(2,2,2-trifluoroethoxy)benzoyl)oxy]-3-methoxyphenyl}acrylate
2-(3,5-Diaminophenyl)ethyl (2E) 3-{4-[(4-(4,4,4-trifluorobutoxy)benzoyl)oxy]-3-methoxyphenyl}acrylate
2-(3,5-Diaminophenyl)ethyl (2E) 3-{4-[(4-(5,5,5-trifluoropentyloxy)benzoyl)oxy]-3-methoxyphenyl}acrylate
2-(3,5-Diaminophenyl)ethyl (2E) 3-{4-[(4-(4,4,5,5,5-pentafluoropentoxy)benzoyl)oxy]-3-methoxyphenyl}acrylate
2-(3,5-Diaminophenyl)ethyl (2E) 3-{4-[(4-(1,1,2,2-tetrafluoroethoxy)benzoyl)oxy]-3-methoxyphenyl}acrylate
2-(3,5-Diaminophenyl)ethyl (2E) 3-{4-[(4-(4,4,4-trifluorobutanoyl)oxy)benzoyl]oxy}-3-methoxyphenyl}acrylate
2-(3,5-Diaminophenyl)ethyl (2E) 3-{4-[(4-trifluoromethoxyphenoxy)carbonyl]phenyl}acrylate
2-(3,5-Diaminophenyl)ethyl (2E) 3-{4-[(4-(2,2,2-trifluoroethoxy)phenoxy)carbonyl]phenyl}acrylate
2-(3,5-Diaminophenyl)ethyl (2E) 3-{4-[(4-(4,4,4-trifluorobutoxy)phenoxy)carbonyl]phenyl}acrylate
2-(3,5-Diaminophenyl)ethyl (2E) 3-{4-[(4-(5,5,5-trifluoropentyloxy)phenoxy)carbonyl]phenyl}acrylate
2-(3,5-Diaminophenyl)ethyl (2E) 3-{4-[(4-(4,4,5,5,5-pentafluoropentoxy)phenoxy)carbonyl]phenyl}acrylate
2-(3,5-Diaminophenyl)ethyl (2E) 3-{4-[(4-(1,1,2,2-tetrafluoroethoxy)phenoxy)carbonyl]phenyl}acrylate
2-(2,5-Diaminophenyl)ethyl (2E) 3-{4-[(4-trifluoromethoxybenzoyl)oxy]phenyl}acrylate
2-(2,5-Diaminophenyl)ethyl (2E) 3-{4-[(4-(2,2,2-trifluoroethoxy)benzoyl)oxy]phenyl}acrylate
2-(2,5-Diaminophenyl)ethyl (2E) 3-{4-[(4-(4,4,4-trifluorobutoxy)benzoyl)oxy]phenyl}acrylate
2-(2,5-Diaminophenyl)ethyl (2E) 3-{4-[(4-(5,5,5-trifluoropentyloxy)benzoyl)oxy]phenyl}acrylate
2-(2,5-Diaminophenyl)ethyl (2E) 3-{4-[(4-(4,4,5,5,5-pentafluoropentoxy)benzoyl)oxy]phenyl}acrylate
2-(2,5-Diaminophenyl)ethyl (2E) 3-{4-[(4-(1,1,2,2-tetrafluoroethoxy)benzoyl)oxy]phenyl}acrylate
2-(2,5-Diaminophenyl)ethyl (2E) 3-{4-[(4-(4,4,4-trifluorobutanoyl)oxy)benzoyl]oxy}phenyl}acrylate
2-(2,4-Diaminophenyl)ethyl (2E) 3-{4-[(4-trifluoromethoxybenzoyl)oxy]phenyl}acrylate
2-(2,4-Diaminophenyl)ethyl (2E) 3-{4-[(4-(2,2,2-trifluoroethoxy)benzoyl)oxy]phenyl}acrylate
2-(2,4-Diaminophenyl)ethyl (2E) 3-{4-[(4-(4,4,4-trifluorobutoxy)benzoyl)oxy]phenyl}acrylate
2-(2,4-Diaminophenyl)ethyl (2E) 3-{4-[(4-(5,5,5-trifluoropentyloxy)benzoyl)oxy]phenyl}acrylate
2-(2,4-Diaminophenyl)ethyl (2E) 3-{4-[(4-(2,2,3,3,3-pentafluoropropyloxy)benzoyl)oxy]phenyl}acrylate
2-(2,4-Diaminophenyl)ethyl (2E) 3-{4-[(4-(2,2,3,4,4,4-hexafluorobutoxy)benzoyl)oxy]phenyl}acrylate
2-(2,4-Diaminophenyl)ethyl (2E) 3-{4-[(4-(3,3,4,4,5,5,6,6,6-nonafluorohexyloxy)benzoyl)oxy]phenyl}acrylate
2-(2,4-Diaminophenyl)ethyl (2E) 3-{4-[(4-(1,1,2,2-tetrafluoroethoxy)benzoyl)oxy]phenyl}acrylate
2-(2,4-Diaminophenyl)ethyl (2E) 3-{4-[(4-(1,1,2,2-tetrafluoropropoxy)benzoyl)oxy]phenyl}acrylate
2-(2,4-Diaminophenyl)ethyl (2E) 3-{4-[(4-(4,4,5,5,6,6,6-heptafluorohexyloxy)benzoyl)oxy]phenyl}acrylate
2-(2,4-Diaminophenyl)ethyl (2E) 3-{4-[(4-(5,5,6,6,6-pentafluorohexyl)benzoyl)oxy]phenyl}acrylate
2-(2,4-Diaminophenyl)ethyl (2E) 3-{4-[(4-(4,4,4-trifluorobutanoyl)oxy)benzoyl]oxy}phenyl}acrylate
2-(2,4-Diaminophenyl)ethyl (2E) 3-{4-[(3-fluoro-4-(4,4,4-trifluorobutoxy)benzoyl) oxy]phenyl}acrylate
2-(2,4-Diaminophenyl)ethyl (2E) 3-{4-[(4-(5,5,5-trifluoropentoxy)benzoyl)oxy]phenyl}acrylate
2-(2,4-Diaminophenyl)ethyl (2E) 3-{4-[(3,4-di(4,4,4-trifluorobutoxy)benzoyl)oxy]phenyl}acrylate
2-(2,4-Diaminophenyl)ethyl (2E) 3-{4-[(4-(4,4,5,5,5-pentafluoropentoxy carbonyl)phenoxy)carbonyl]phenyl}acrylate
2-(2,4-Diaminophenyl)ethyl (2E) 3-{4-[(4-[(4,4,4-trifluorobutoxy)carbonyl]amino) benzoyl]oxy}phenyl}acrylate.
2-(2,4-Diaminophenyl)ethyl (2E) 3-{4-[(4-[(4,4,5,5,5-pentafluoropentoxy)carbonyl]amino)benzoyl]oxy}phenyl}acrylate
2-(2,4-Diaminophenyl)ethyl (2E) 3-{4-[(3-methoxy-4-trifluoromethoxybenzoyl)oxy]phenyl}acrylate
2-(2,4-Diaminophenyl)ethyl (2E) 3-{4-[(3-methoxy 4-(2,2,2-trifluoroethoxy)benzoyl)oxy]phenyl}acrylate
2-(2,4-Diaminophenyl)ethyl (2E) 3-{4-[(3-methoxy 4-(4,4,4,-trifluorobutoxy)benzoyl)oxy]phenyl}acrylate
2-(2,4-Diaminophenyl)ethyl (2E) 3-{4-[(3-methoxy 4-(5,5,5-trifluoropentyloxy)benzoyl)oxy]phenyl}acrylate
2-(2,4-Diaminophenyl)ethyl (2E) 3-{4-[(3-methoxy 4-(4,4,5,5,5-pentafluoropentoxy)benzoyl)oxy]phenyl}acrylate
2-(2,4-Diaminophenyl)ethyl (2E) 3-{4-[(3-methoxy 4-(1,1,2,2-tetrafluoroethoxy)benzoyl)oxy]phenyl}acrylate
2-(2,4-Diaminophenyl)ethyl (2E) 3-{4-[(3-methoxy 4-(4,4,4-trifluorobutanoyl)oxy)benzoyl]oxy}phenyl}acrylate
2-(2,4-Diaminophenyl)ethyl (2E) 3-{4-[(4-(4,4,4-trifluorobutoxy)benzoyl)oxy]-3-methoxy phenyl}acrylate
2-(2,4-Diaminophenyl)ethyl (2E) 3-{4-[(4-(5,5,5-trifluoropentyloxy)benzoyl)oxy]-3-methoxyphenyl}acrylate
2-(2,4-Diaminophenyl)ethyl (2E) 3-{-4[(4-(4,4,5,5,5-pentafluoropentoxy)benzoyl)oxy]-3-methoxyphenyl}acrylate
2-(2,4-Diaminophenyl)ethyl (2E) 3-{4-[(4-(1,1,2,2-tetrafluoroethoxy)benzoyl)oxy]-3-methoxyphenyl}acrylate
2-(2,4-Diaminophenyl)ethyl (2E) 3-{4-[(4-(4,4,4-trifluorobutanoyl)oxy)benzoyl]oxy}-3-methoxyphenyl}acrylate
2-(2,4-Diaminophenyl)ethyl (2E) 3-{4-[(4,4,4-trifluorobutoxy)phenoxy)carbonyl]phenyl}acrylate
2-(2,4-Diaminophenyl)ethyl (2E) 3-{4-[(4-(5,5,5-trifluoropentyloxy)phenoxy)carbonyl]phenyl}acrylate
2-(2,4-Diaminophenyl)ethyl (2E) 3-{4-[(4-(4,4,5,5,5-pentafluoropentoxy)phenoxy)carbonyl]phenyl}acrylate
2-(2,4-Diaminophenyl)ethyl (2E) 3-{4-[(4-(1,1,2,2-tetrafluoroethoxy)phenoxy)carbonyl]phenyl}acrylate
3-(3,5-Diaminophenyl)propyl (2E) 3-{4-[(4-trifluoromethoxybenzoyl)oxy]phenyl}acrylate
3-(3,5-Diaminophenyl)propyl (2E) 3-{4-[(4-(2,2,2-trifluoroethoxy)benzoyl)oxy]phenyl}acrylate
3-(3,5-Diaminophenyl)propyl (2E) 3-{4-[(4-(4,4,4-trifluorobutoxy)benzoyl)oxy]phenyl}acrylate
3-(3,5-Diaminophenyl)propyl (2E) 3-{4-[(4-(5,5,5-trifluoropentyloxy)benzoyl)oxy]phenyl}acrylate
3-(3,5-Diaminophenyl)propyl (2E) 3-{4-[(4-(4,4,5,5,5-pentafluoropentoxy)benzoyl)oxy]phenyl}acrylate
3-(3,5-Diaminophenyl)propyl (2E) 3-{4-[(4-(1,1,2,2-tetrafluoroethoxy)benzoyl)oxy]phenyl}acrylate
3-(3,5-Diaminophenyl)propyl (2E) 3-{4-[(4-(4,4,4-trifluorobutanoyl)oxy)benzoyl]oxy}phenyl}acrylate 3-(2,4-Diaminophenyl)propyl (2E) 3-{4-[(4-(4,4,4-trifluorobutoxy)benzoyl)oxy]phenyl}acrylate
3-(2,4-Diaminophenyl)propyl (2E) 3-{4-[(4-(4,4,5,5,5-pentafluoropentoxy)benzoyl)oxy]phenyl}acrylate
3-(2,4-Diaminophenyl)propyl (2E) 3-{4-[(4-(1,1,2,2-tetrafluoroethoxy)benzoyl)oxy]phenyl}acrylate
3-(2,4-Diaminophenyl)propyl (2E) 3-{4-[(4-(4,4,4-trifluorobutanoyl)oxy)benzoyl]oxy}phenyl}acrylate
6-(2,4-Diaminophenyl)hexyl (2E) 3-{4-[(4-(4,4,4-trifluorobutoxy)benzoyl)oxy]phenyl}acrylate
6-(2,4-Diaminophenyl)hexyl (2E) 3-{4-[(4-(4,4,5,5,5-pentafluoropentoxy)benzoyl)oxy]phenyl}acrylate
6-(2,4-Diaminophenyl)hexyl (2E) 3-{4-[(4-(1,1,2,2-tetrafluoroethoxy)benzoyl)oxy]phenyl}acrylate

### EXAMPLE 5

### Synthesis

### Preparation of 2,2-bis(4-aminobenzyl)-1,3 di[(2E)-3-{4-[(4-(4,4,4-trifluorobutoxy)benzoyl)oxy]phenyl}prop-2-enoyl]propanediol

### 5.1 Preparation of 2,2-dimethyl-5,5-bis(4-nitrobenzyl)-1,3-dioxane-4,6-dione

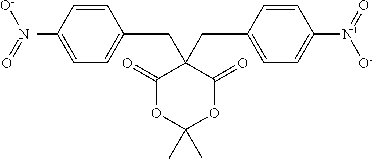

15.0 g (69.4 mmol) of 4-nitrobenzylbromide and 5.00 g (34.7 mmol) of Meldrum's acid are dissolved in 100 ml 2-butanone. 4.40 g (104.1 mmol) potassium carbonate are added, the resulting suspension is heated to 50° C. and allowed to react for 2.5 hours. After cooling to room temperature, 100 ml water are added. The product is collected by filtration and washed with a lot of water. 12.3 g (85%) of 2,2-dimethyl-5,5-bis(4-nitrobenzyl)-1,3-dioxane-4,6-dione as yellowish powder is used without further purification.

### 5.2 Preparation of 2,2-bis(4-nitrobenzyl)malonic acid

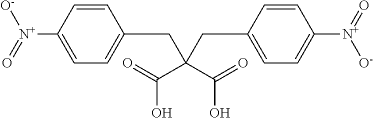

2.185 g (52.07 mmol) of lithium hydroxide are added to a suspension of 10.79 g (26.04 mmol) of 2,2-dimethyl-5,5-bis(4-nitrobenzyl)-1,3-dioxane-4,6-dione and 110 ml mixture of tetrahydrofurane:water 9:1. The mixture is subsequently allowed to react for 21.5 hours at 25° C., added to 500 ml water and acidified to pH=1 with 20 ml hydrochloric acid 3N. The mixture is partitioned between water and ethyl acetate; the organic phase is washed repeatedly with water, dried over sodium sulfate, filtered and concentrated by rotary evaporation. The residue 9.54 g (98%) of 2,2-bis(4-nitrobenzyl)malonic acid as white powder is used without further purification.

### 5.3 Preparation of 2,2-bis(4-nitrobenzyl)-1,3-propandiol

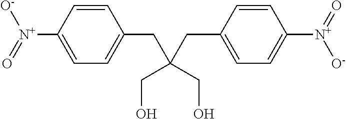

4.00 g (10.69 mmol) 2,2-bis(4-nitrobenzyl)malonic acid are dissolved in 40 ml tetrahydrofuran and added dropwise in a the course of 2 hours to 64.1 ml (64.1 mmol) of a borane-tetrahydrofuran complex 1.0 M solution in tetrahydrofuran. After 19 hours at 25° C., 50 ml water are carefully added. The reaction mixture is then partitioned between ethyl acetate and water; the organic phase is washed repeatedly with water, dried over sodium sulfate, filtered and concentrated by rotary evaporation. The residue, 3.77 g (97%) of 2,2-bis(4-nitrobenzyl)-1,3-propandiol as white powder is used without further purification.

### 5.4 Preparation 2,2-bis(4-nitrobenzyl)-1,3 di[(2E)-3-{4-[(4-(4,4,4-trifluorobutoxy)benzoyl)oxy]phenyl}prop-2-enoyl]propanediol

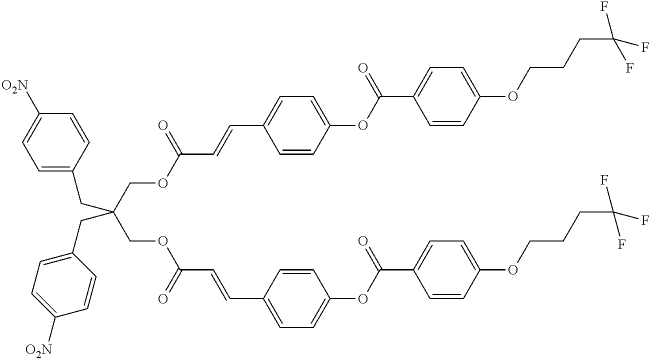

1.76 g (5.07 mmol) of 2,2-bis(4-nitrobenzyl)-1,3-propandiol, 4.00 g (10.14 mmol) of (2E)-3-(4-{[4-(4,4,4-trifluorobutoxy)benzoyl]oxy}phenyl)acrylic acid, 124 mg (1.01 mmol) of 4-Dimethylaminopyridine are dissolved in 100 ml of dichloromethane. 2.14 g (11.16 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation. Chromatography of the residue on 150 g silica gel using toluene:ethyl acetate 9:1 as eluant to yield 2.20 g 2,2-bis(4-nitrobenzyl)-1,3 di[(2E)-3-{4-[(4-(4,4,4-trifluorobutoxy)benzoyl)oxy]phenyl}prop-2-enoyl]propanediol as white crystals.

### 5.5 Preparation of 2,2-bis(4-aminobenzyl)-1,3 di[(2E)-3-{4-[(4-(4,4,4-trifluorobutoxy)benzoyl)oxy]phenyl}prop-2-enoyl]propanediol

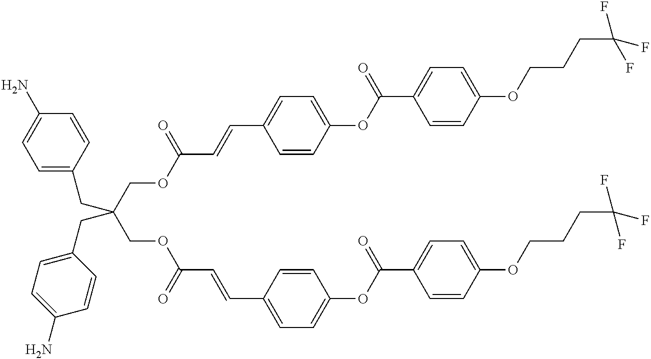

2.20 g (2.00 mol) of 2,2-bis(4-nitrobenzyl)-1,3 di[(2E)-3-{4-[(4-(4,4,4-trifluorobutoxy)benzoyl)oxy]phenyl}prop-2-enoyl]propanediol are dissolved in a mixture of 25 ml of N,N-dimethylformamide and 3 ml water. 3.25 g (12.01 mmol) ferric chloride hexahydrate are added. 1.31 g (20.02 mmol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 100 g silica gel using toluene:ethyl acetate 1:1 as eluant and crystallization form ethylacetate:hexane mixture to yield 1.20 g 2,2-bis(4-aminobenzyl)-1,3 di[(2E)-3-{4-[(4-(4,4,4-trifluorobutoxy)benzoyl)oxy]phenyl}prop-2-enoyl]propanediol

The following diamines are synthesized in an analogous manner:
2,2-bis(4-aminobenzyl)-1,3 di[(2E) 3-{4-[(4-trifluoromethoxybenzoyl)oxy]phenyl}prop-2-enoyl]propanediol
2,2-bis(4-aminobenzyl)-1,3 di[(2E) 3-{4-[(4-(2,2,2-trifluoroethoxy)benzoyl)oxy]phenyl}prop-2-enoyl]propanediol
2,2-bis(4-aminobenzyl)-1,3 di[(2E) 3-{4-[(4-(4,4,4-trifluorobutoxy)benzoyl)oxy]phenyl}prop-2-enoyl]propanediol
2,2-bis(4-aminobenzyl)-1,3 di[(2E) 3-{4-[(4-(5,5,5-trifluoropentyloxy)benzoyl)oxy]phenyl}prop-2-enoyl]propanediol
2,2-bis(4-aminobenzyl)-1,3 di[(2E) 3-{4-[(4-(4,4,5,5,5-pentafluoropentoxy)benzoyl)oxy]phenyl}prop-2-enoyl]propanediol
2,2-bis(4-aminobenzyl)-1,3 di[(2E) 3-{4-[(4-(1,1,2,2-tetrafluoroethoxy)benzoyl)oxy]phenyl}prop-2-enoyl]propanediol
2,2-bis(4-aminobenzyl)-1,3 di[(2E) 3-{4-[(4-(4,4,4-trifluorobutanoyl)oxy)benzoyl]oxy}phenyl}prop-2-enoyl]propanediol
2,2-bis(4-aminobenzyl)-1,3 di[(2E) 3-{4-[(3-methoxy-4-trifluoromethoxybenzoyl)oxy]phenyl}prop-2-enoyl]propanediol
2,2-bis(4-aminobenzyl)-1,3 di[(2E) 3-{4-[(3-methoxy 4-(2,2,2-trifluoroethoxy)benzoyl)oxy]phenyl}prop-2-enoyl]propanediol
2,2-bis(4-aminobenzyl)-1,3 di[(2E) 3-{4-[(3-methoxy 4-(4,4,4-trifluorobutoxy)benzoyl)oxy]phenyl}prop-2-enoyl]propanediol
2,2-bis(4-aminobenzyl)-1,3 di[(2E) 3-{4-[(3-methoxy 4-(5,5,5-trifluoropentyloxy)benzoyl)oxy]phenyl}prop-2-enoyl]propanediol
2,2-bis(4-aminobenzyl)-1,3 di[(2E) 3-{4-[(3-methoxy 4-(4,4,5,5,5-pentafluoropentoxy)benzoyl)oxy]phenyl}prop-2-enoyl]propanediol
2,2-bis(4-aminobenzyl)-1,3 di[(2E) 3-{4-[(3-methoxy 4-(1,1,2,2-tetrafluoroethoxy)benzoyl)oxy]phenyl}prop-2-enoyl]propanediol
2,2-bis(4-aminobenzyl)-1,3 di[(2E) 3-{4-[(3-methoxy 4-(4,4,4-trifluorobutanoyl)oxy)benzoyl]oxy}phenyl}prop-2-enoyl]propanediol
2,2-bis(4-aminobenzyl)-1,3 di[(2E) 3-{4-[(4-trifluoromethoxybenzoyl)oxy]-3-methoxyphenyl}prop-2-enoyl]propanediol
2,2-bis(4-aminobenzyl)-1,3 di[(2E) 3-{4-[(4-(2,2,2-trifluoroethoxy)benzoyl)oxy]-3-methoxyphenyl}prop-2-enoyl]propanediol
2,2-bis(4-aminobenzyl)-1,3 di[(2E) 3-{4-[(4-(4,4,4-trifluorobutoxy)benzoyl)oxy]-3-methoxy phenyl}prop-2-enoyl]propanediol
2,2-bis(4-aminobenzyl)-1,3 di[(2E) 3-{4-[(4-(5,5,5-trifluoropentyloxy)benzoyl)oxy]-3-methoxyphenyl}prop-2-enoyl]propanediol
2,2-bis(4-aminobenzyl)-1,3 di[(2E) 3-{4-[(4-(4,4,5,5,5-pentafluoropentoxy)benzoyl)oxy]-3-methoxyphenyl}prop-2-enoyl]propanediol
2,2-bis(4-aminobenzyl)-1,3 di[(2E) 3-{4-[(4-(1,1,2,2-tetrafluoroethoxy)benzoyl)oxy]-3-methoxyphenyl}prop-2-enoyl]propanediol
2,2-bis(4-aminobenzyl)-1,3 di[(2E) 3-{4-[(4-(4,4,4-trifluorobutanoyl)oxy)benzoyl]oxy}-3-methoxyphenyl}prop-2-enoyl]propanediol
2,2-bis(4-aminobenzyl)-1,3 di[(2E) 3-{4-[(4-trifluoromethoxyphenoxy)carbonyl]phenyl}prop-2-enoyl]propanediol
2,2-bis(4-aminobenzyl)-1,3 di[(2E) 3-{4-[(4-(2,2,2-trifluoroethoxy)phenoxy)carbonyl]phenyl}prop-2-enoyl]propanediol
2,2-bis(4-aminobenzyl)-1,3 di[(2E) 3-{4-[(4-(4,4,4-trifluorobutoxy)phenoxy)carbonyl]phenyl}prop-2-enoyl]propanediol
2,2-bis(4-aminobenzyl)-1,3 di[(2E) 3-{4-[(4-(5,5,5-trifluoropentyloxy)phenoxy)carbonyl]phenyl}prop-2-enoyl]propanediol
2,2-bis(4-aminobenzyl)-1,3 di[(2E) 3-{4-[(4-(4,4,5,5,5-pentafluoropentoxy)phenoxy)carbonyl]phenyl}prop-2-enoyl]propanediol
2,2-bis(4-aminobenzyl)-1,3 di[(2E) 3-{4-[(4-(1,1,2,2-tetrafluoroethoxy)phenoxy)carbonyl]phenyl}prop-2-enoyl]propanediol

### EXAMPLE 6

### Synthesis

### Preparation of 6-{[((2E)-3-{4-[(4-(4,4,4-trifluorobutoxy)benzoyl)oxy]phenyl}prop-2-enoyl)oxy]}hexyl 3,5-Diamino-4-[6-{[((2E)-3-{4-[(4-(4,4,4-trifluorobutoxy)benzoyl)oxy]phenyl}prop-2-enoyl)oxy]}hexyloxy]benzoate

### 6.1 Preparation of 6-{[((2E)-3-{4-[(4-(4,4,4-trifluorobutoxy)benzoyl)oxy]phenyl}prop-2-enoyl)oxy]}hexyl 3,5-dinitro-4-[6-{[((2E)-3-{4-[(4-(4,4,4-trifluorobutoxy)benzoyl)oxy]phenyl}prop-2-enoyl)oxy]}hexyloxy]benzoate

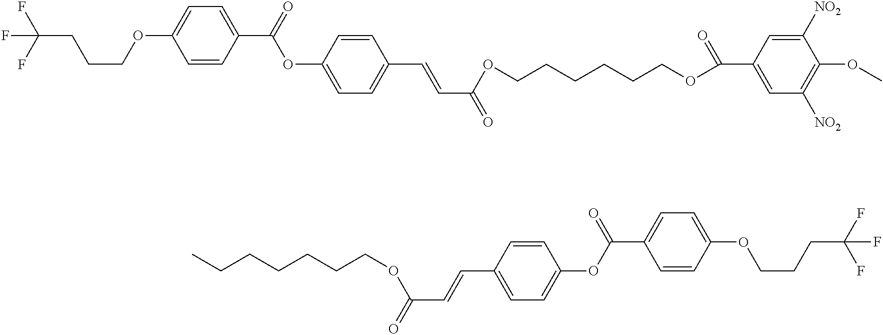

6.50 g (11.67 mmol) of 6-hydroxyhexyl 4-(6-hydroxyhexyloxy)-3,5-dinitrobenzoate, 9.67 g (24.53 mmol) of (2E)-3-(4-{[4-(4,4,4-trifluorobutoxy)benzoyl]oxy}phenyl)acrylic acid, 290 mg (2.34 mmol) of 4-Dimethylaminopyridine are dissolved in 100 ml of dichloromethane. 5.14 g (26.87 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation. Chromatography of the residue on 500 g silica gel using toluene:ethyl acetate 95:5 as eluant and crystallization form ethyl acetate:hexane mixture to yield 7.70 g of 6-{[((2E)-3-{4-[(4-(4,4,4-trifluorobutoxy)benzoyl)oxy]phenyl}prop-2-enoyl)oxy]}hexyl 3,5-dinitro-4-[6-{[((2E)-3-{4-[(4-(4,4,4-trifluorobutoxy)benzoyl)oxy]phenyl}prop-2-enoyl)oxy]}hexyloxy]benzoate as yellow crystals

### 6.2 Preparation of 6-{[((2E)-3-{4-[(4-(4,4,4-trifluorobutoxy)benzoyl)oxy]phenyl}prop-2-enoyl)oxy]}hexyl 3,5-Diamino-4-[6-{[((2E)-3-{4-[(4-(4,4,4-trifluorobutoxy)benzoyl)oxy]phenyl}prop-2-enoyl)oxy]}hexyloxy]benzoate

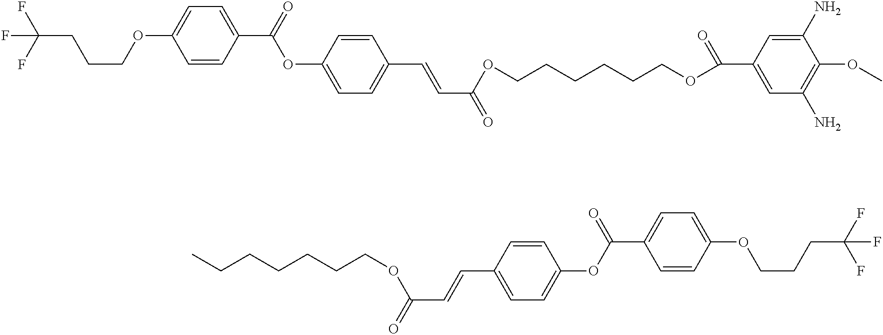

7.70 g (6.5 mol) of 6-{[((2E)-3-{4-[(4-(4,4,4-trifluorobutoxy)benzoyl)oxy]phenyl}prop-2-enoyl)oxy]}hexyl 3,5-dinitro-4-[6-{[((2E)-3-{4-[(4-(4,4,4-trifluorobutoxy)benzoyl)oxy]phenyl}prop-2-enoyl)oxy]}hexyloxy]benzoate are dissolved in a mixture of 90 ml of N,N-dimethylformamide and 7 ml water. 10.6 g (39.2 mmol) ferric chloride hexahydrate are added. 4.27 g (65.36 mmol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 200 g silica gel using toluene:ethyl acetate 2:1 as eluant and crystallization form methanol:ethyl acetate mixture to yield 4.92 g 6-{[((2E)-3-{4-[(4-(4,4,4-trifluorobutoxy)benzoyl)oxy]phenyl}prop-2-enoyl)oxy]}hexyl 3,5-Diamino-4-[6-{[((2E)-3-{4-[(4-(4,4,4-trifluorobutoxy)benzoyl)oxy]phenyl}prop-2-enoyl)oxy]}hexyloxy]benzoate as colorless crystals.

### EXAMPLE 7

### Synthesis

### 7.1 Preparation of 4,4′-Dinitro-1,1′-biphenyl-2,2′-dicarboxylic acid

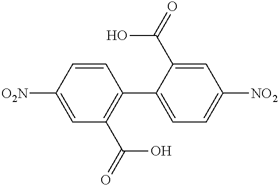

30.0 g (120.13 mmol) Diphenic acid are dissolved at room temperature in 469 g (4.59 mol) concentrated sulfuric acid (96%). The solution is cooled to −15° C. and a mixture of 92.4 g (1.011 mol) concentrated nitric acid (69%) and 12.0 g (0.117 mol) concentrated sulfuric acid (96%) is added slowly so that the mixture temperature is maintained below 0° C. After the addition the solution is allowed to react at room temperature for 24 h.

After the mixture is poured onto crushed ice, the precipitate that formed i collected by filtration, washed with water and dried at room temperature under vacuum for 10 h.

### 7.2 Preparation of 2,2′-bis(hydroxymethyl-4,4′-Dinitro 1,1′-biphenyl

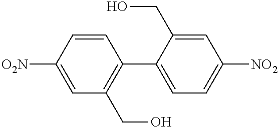

3.6 g (10.83 mmol) 4,4′-Dinitro-1,1′-biphenyl-2,2′-dicarboxylic acid ae dissolved in 25 ml tetrahydrofuran and added dropwise in a the course of 1 hours to 65 ml (65.02 mmol) of a borane-tetrahydrofuran complex 1.0 M solution in tetrahydrofuran. After 19 hours at 25° C., 50 ml water are carefully added. After 1 h the solution is acidified to pH=1-2 with 10 ml 1N HCl solution and allowed to stirred for 30 min. The reaction mixture is then partitioned between ethyl acetate and water; the organic phase is washed repeatedly with water, dried over sodium sulfate, filtered and concentrated by rotary evaporation. The residue, 4.2 g of 2,2′-bis(hydroxymethyl-4,4′-Dinitro 1,1′-biphenyl as white powder is used without further purification.

### 7.3 Preparation of 2,2′-bis[(2E)-3-{4-[(4-(4,4,4-trifluorobutoxy)benzoyl)oxy]phenyl}prop-2-enoyl]methyl 4,4′-Dinitro 1,1′-biphenyl

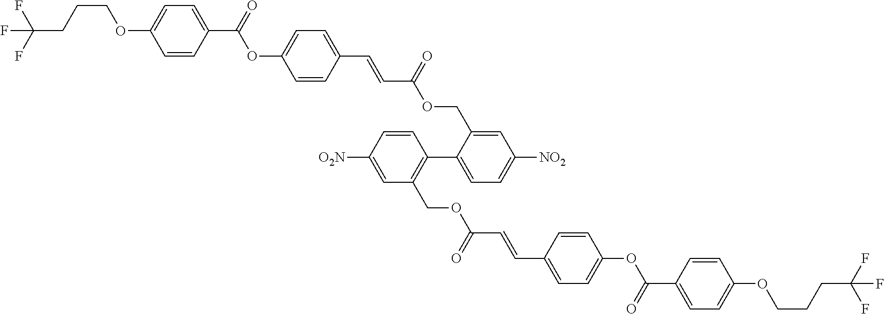

3.92 g (12.8 mmol) of 2,2′-bis(hydroxymethyl-4,4′-Dinitro 1,1′-biphenyl, 13.20 g (33.5 mmol) of (2E)-3-(4-{[4-(4,4,4-trifluorobutoxy)benzoyl]oxy}phenyl)acrylic acid prepared according to example 1, 0.630 mg (5.15 mmol) of 4-Dimethylaminopyridine are dissolved in 200 ml of dichloromethane. 6.91 g (11.16 mmol) of N,N′-dicyclohexylcarbodiimide are added at 0° C. The solution is stirred for 2 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation. Chromatography of the residue on 150 g silica gel using toluene:ethyl acetate 9:1 as eluant to yield 12.0 g 2,2′-bis[(2E)-3-{4-[(4-(4,4,4-trifluorobutoxy)benzoyl)oxy]phenyl}prop-2-enoyl]methyl 4,4′-Dinitro 1,1′-biphenyl as white crystals.

### 7.4 Preparation 2,2′-bis[(2E)-3-{4-[(4-(4,4,4-trifluorobutoxy)benzoyl)oxy]phenyl}prop-2-enoyl]methyl 4,4′-Diamino 1,1′-biphenyl

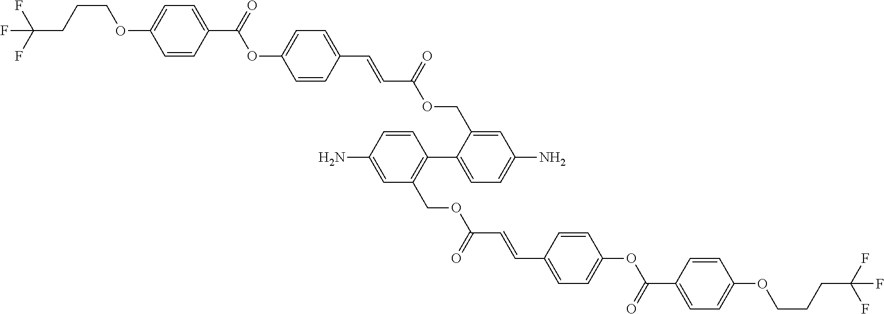

2.27 g (2.14 mol) of 2,2′-bis[(2E)-3-{4-[(4-(4,4,4-trifluorobutoxy)benzoyl)oxy]phenyl}prop-2-enoyl]methyl 4,4′-Dinitro 1,1′-biphenyl are dissolved in a mixture of 40 ml of N,N-dimethylformamide and 3 ml water. 3.48 g (12.8 mmol) ferric chloride hexahydrate are added. 1.40 g (21.4 mmol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 100 g silica gel using toluene:ethyl acetate 7:3 as eluant yield 1.74 g 2,2′-bis[(2E)-3-{4-[(4-(4,4,4-trifluorobutoxy)benzoyl)oxy]phenyl}prop-2-enoyl]methyl 4,4′-Diamino 1,1′-biphenyl as yellowish crystals.

2,2′-bis[(2E) 3-{4-[(4-(4,4,5,5,5-pentafluoropentoxy)benzoyl)oxy]phenyl}prop-2-enoyl]methyl 4,4′-diamino 1,1′-biphenyl is prepared analogous to example 7 using (2E) 3-{4-[(4-(4,4,5,5,5-pentafluoropentyloxy)benzoyl)oxy]phenyl}acrylic acid.

The following diamines are synthesized in an analogous manner:
2,2′-bis[(2E) 3-{4-[(4-trifluoromethoxybenzoyl)oxy]phenyl}prop-2-enoyl]methyl 4,4′-diamino 1,1′-biphenyl
2,2′-bis[(2E) 3-{4-[(4-(2,2,2-trifluoroethoxy)benzoyl)oxy]phenyl}prop-2-enoyl]methyl
4,4′-diamino 1,1′-biphenyl
2,2′-bis[(2E) 3-{4-[(4-(4,4,4-trifluorobutoxy)benzoyl)oxy]phenyl}prop-2-enoyl]methyl
4,4′-diamino 1,1′-biphenyl
2,2′-bis[(2E) 3-{4-[(4-(5,5,5-trifluoropentyloxy)benzoyl)oxy]phenyl}prop-2-enoyl]methyl
4,4′-diamino 1,1′-biphenyl
2,2′-bis[(2E) 3-{4-[(4-(1,1,2,2-tetrafluoroethoxy)benzoyl)oxy]phenyl}prop-2-enoyl]methyl 4,4′-diamino 1,1′-biphenyl
2,2′-bis[(2E) 3-{4-[(4-(4,4,4-trifluorobutanoyl)oxy)benzoyl]oxy}phenyl]prop-2-enoyl]methyl 4,4′-diamino 1,1′-biphenyl
2,2′-bis[(2E) 3-{4-[(3-methoxy-4-trifluoromethoxybenzoyl)oxy]phenyl}prop-2-enoyl]methyl 4,4′-diamino 1,1′-biphenyl
2,2′-bis[(2E) 3-{4-[(3-methoxy 4-(2,2,2-trifluoroethoxy)benzoyl)oxy]phenyl}prop-2-enoyl]methyl 4,4′-diamino 1,1′-biphenyl
2,2′-bis[(2E) 3-{4-[(3-methoxy 4-(4,4,4-trifluorobutoxy)benzoyl)oxy]phenyl}prop-2-enoyl]methyl 4,4′-diamino 1,1′-biphenyl
2,2′-bis[(2E) 3-{4-[(3-methoxy 4-(5,5,5-trifluoropentyloxy)benzoyl)oxy]phenyl}prop-2-enoyl]methyl 4,4′-diamino 1,1′-biphenyl
2,2′-bis[(2E) 3-{4-[(3-methoxy 4-(4,4,5,5,5-pentafluoropentoxy)benzoyl)oxy]phenyl}prop-2-enoyl]methyl 4,4′-diamino 1,1′-biphenyl
2,2′-bis[(2E) 3-{4-[(3-methoxy 4-(1,1,2,2-tetrafluoroethoxy)benzoyl)oxy]phenyl}prop-2-enoyl]methyl 4,4′-diamino 1,1′-biphenyl
2,2′-bis[(2E) 3-{4-[(3-methoxy 4-(4,4,4-trifluorobutanoyl)oxy)benzoyl]oxy}phenyl]prop-2-enoyl]methyl 4,4′-diamino 1,1′-biphenyl
2,2′-bis[(2E) 3-{4-[(4-trifluoromethoxybenzoyl)oxy]-3-methoxyphenyl}prop-2-enoyl]methyl 4,4′-diamino 1,1′-biphenyl
2,2′-bis[(2E) 3-{4-[(4-(2,2,2-trifluoroethoxy)benzoyl)oxy]-3-methoxyphenyl]prop-2-enoyl]methyl 4,4′-diamino 1,1′-biphenyl
2,2′-bis[(2E) 3-{4-[(4-(4,4,4-trifluorobutoxy)benzoyl)oxy]-3-methoxy phenyl}prop-2-enoyl]methyl 4,4′-diamino 1,1′-biphenyl
2,2′-bis[(2E) 3-{4-[(4-(5,5,5-trifluoropentyloxy)benzoyl)oxy]-3-methoxyphenyl}prop-2-enoyl]methyl 4,4′-diamino 1,1′-biphenyl
2,2′-bis[(2E) 3-{4-[(4-(4,4,5,5,5-pentafluoropentoxy)benzoyl)oxy]-3-methoxyphenyl]prop-2-enoyl]methyl 4,4′-diamino 1,1′-biphenyl
2,2′-bis[(2E) 3-{4-[(4-(1,1,2,2-tetrafluoroethoxy)benzoyl)oxy]-3-methoxyphenyl}prop-2-enoyl]methyl 4,4′-diamino 1,1′-biphenyl
2,2′-bis[(2E) 3-{4-[(4-(4,4,4-trifluorobutanoyl)oxy)benzoyl]oxy}-3-methoxyphenyl]prop-2-enoyl]methyl 4,4′-diamino 1,1′-biphenyl
2,2′-bis[(2E) 3-{4-[(4-trifluoromethoxyphenoxy)carbonyl]phenyl}prop-2-enoyl]methyl
4,4′-diamino 1,1′-biphenyl 2,2′-bis[(2E) 3-{4-[(4-(2,2,2-trifluoroethoxy)phenoxy)carbonyl]phenyl}prop-2-enoyl]methyl 4,4′-diamino 1,1′-biphenyl
2,2′-bis[(2E) 3-{4-[(4-(4,4,4-trifluorobutoxy)phenoxy)carbonyl]phenyl}prop-2-enoyl]methyl 4,4′-diamino 1,1′-biphenyl
2,2′-bis[(2E) 3-{4-[(4-(5,5,5-trifluoropentyloxy)phenoxy)carbonyl]phenyl}prop-2-enoyl methyl 4,4′-diamino 1,1′-biphenyl
2,2′-bis[(2E) 3-{4-[(4-(4,4,5,5,5-pentafluoropentoxy)phenoxy)carbonyl]phenyl]prop-2-enoyl]methyl 4,4′-diamino 1,1′-biphenyl
2,2′-bis[(2E) 3-{4-[(4-(1,1,2,2-tetrafluoroethoxy)phenoxy)carbonyl]phenyl]prop-2-enoyl]methyl 4,4′-diamino 1,1′-biphenyl

### EXAMPLE 8

### Synthesis

### Preparation of 2-(2,4-Diaminophenyl)-1,3 di[(2E)-3-{4-[(4-(4,4,4-trifluorobutoxy)benzoyl)oxy]phenyl}prop-2-enoyl]propanediol

### 8.1 Preparation of 2-(2,4-Dinitrophenyl)-1,3 di[(2E)-3-{4-[(4-(4,4,4-trifluorobutoxy)benzoyl)oxy]phenyl}prop-2-enoyl]propanediol

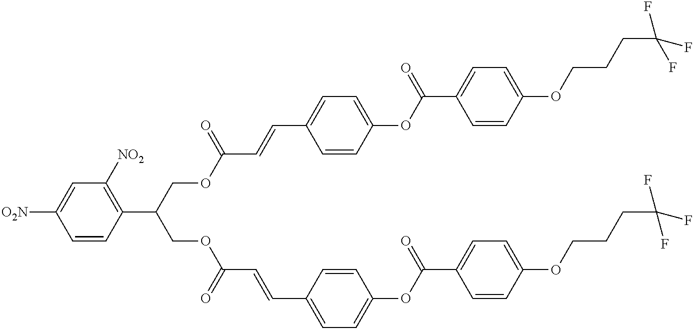

2.90 g (12.0 mmol) of 2-(4-nitrophenyl)-1,3-propandiol, 9.54 g (24.2 mmol) of (2E)-3-(4-{[4-(4,4,4-trifluorobutoxy)benzoyl]oxy}phenyl)acrylic acid. 296 mg (2.42 mmol) of 4-Dimethylaminopyridine are dissolved in 100 ml of dichloromethane. 9.20 g (49.0 mmol) of N-(3-Dimethylaminopropyl)-N′-ethylcarbodiimide hydrochloride (EDC hydrochloride) are added at 0° C. The solution is stirred for 1 h at 0° C. and allowed to stir at room temperature overnight. After 22 hours at room temperature the reaction mixture is partitioned between dichloromethane and water. The organic phase is washed repeatedly with water, dried over sodium sulphate, filtered and concentrated by rotary evaporation. Chromatography of the residue on 600 g silica gel using toluene:ethyl acetate 9:1 as eluant to yield 7.60 g 2-(4-nitrophenyl)-1,3 di[(2E)-3-{4-[(4-(4,4,4-trifluorobutoxy)benzoyl)oxy]phenyl}prop-2-enoyl]propanediol as white crystals.

### 8.2 Preparation of 2-(2,4-Diaminophenyl)-1,3 di[(2E)-3-{4-[(4-(4,4,4-trifluorobutoxy)benzoyl)oxy]phenyl}prop-2-enoyl]propanediol

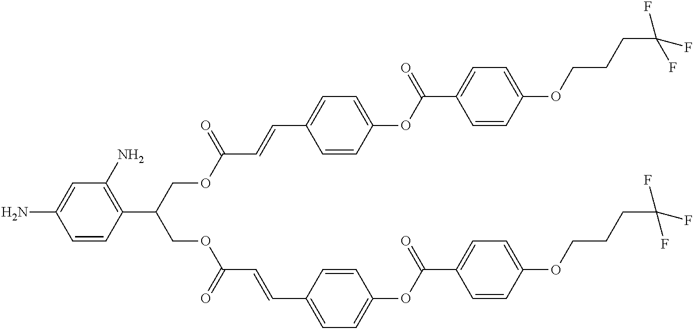

7.60 g (7.64 mmol) of 2-(4-nitrophenyl)-1,3 di[(2E)-3-{4-[(4-(4,4,4-trifluorobutoxy)benzoyl)oxy]phenyl}prop-2-enoyl]propanediol are dissolved in a mixture of 45 ml of N,N-dimethylformamide and 5 ml water. 12.39 g (45.84 mmol) ferric chloride hexahydrate are added. 4.99 g (76.4 mmol) Zinc powder are added portionwise within 40 min. The mixture is allowed to react for 2 hours. The reaction mixture is then partitioned between ethyl acetate and water and filtered. The organic phase is washed repeatedly with water, dried over sodium sulfate, filtered and concentrated by rotary evaporation. Chromatography of the residue on 1000 g silica gel using toluene:ethyl acetate 1:1 as eluant and crystallization form ethylacetate:hexane mixture to yield 4.30 g of 2-(2,4-Diaminophenyl)-1,3 di[(2E)-3-{4-[(4-(4,4,4-trifluorobutoxy)benzoyl)oxy]phenyl}prop-2-enoyl]propanediol.

The following diamines are synthesized in an analogous manner:
2-(2,4-Diaminophenyl)-1,3 di[(2E) 3-{4-[(4-trifluoromethoxybenzoyl)oxy]phenyl}prop-2-enoyl]propanediol
2-(2,4-Diaminophenyl)-1,3 di[(2E) 3-{4-[(4-(2,2,2-trifluoroethoxy)benzoyl)oxy]phenyl}prop-2-enoyl]propanediol
2-(2,4-Diaminophenyl)-1,3 di[(2E) 3-{4-[(4-(4,4,4-trifluorobutoxy)benzoyl)oxy]phenyl}prop-2-enoyl]propanediol
2-(2,4-Diaminophenyl)-1,3 di[(2E) 3-{4-[(4-(5,5,5-trifluoropentyloxy)benzoyl)oxy]phenyl}prop-2-enoyl]propanediol
2-(2,4-Diaminophenyl)-1,3 di[(2E) 3-{4-[(4-(4,4,5,5,5-pentafluoropentoxy)benzoyl)oxy]phenyl}prop-2-enoyl]propanediol
2-(2,4-Diaminophenyl)-1,3 di[(2E) 3-{4-[(4-(1,1,2,2-tetrafluoroethoxy)benzoyl)oxy]phenyl}prop-2-enoyl]propanediol
2-(2,4-Diaminophenyl)-1,3 di[(2E) 3-{4-[(4-(4,4,4-trifluorobutanoyl)oxy)benzoyl]oxy}phenyl}prop-2-enoyl]propanediol
2-(2,4-Diaminophenyl)-1,3 di[(2E) 3-{4-[(3-methoxy-4-trifluoromethoxybenzoyl)oxy]phenyl}prop-2-enoyl]propanediol
2-(2,4-Diaminophenyl)-1,3 di[(2E) 3-{4-[(3-methoxy 4-(2,2,2-trifluoroethoxy)benzoyl)oxy]phenyl}prop-2-enoyl]propanediol
2-(2,4-Diaminophenyl)-1,3 di[(2E) 3-{4-[(3-methoxy 4-(4,4,4-trifluorobutoxy)benzoyl)oxy]phenyl}prop-2-enoyl]propanediol
2-(2,4-Diaminophenyl)-1,3 di[(2E) 3-{4-[(3-methoxy 4-(5,5,5-trifluoropentyloxy)benzoyl)oxy]phenyl}prop-2-enoyl]propanediol
2-(2,4-Diaminophenyl)-1,3 di[(2E) 3-{4-[(3-methoxy 4-(4,4,5,5,5-pentafluoropentoxy)benzoyl)oxy]phenyl}prop-2-enoyl]propanediol
2-(2,4-Diaminophenyl)-1,3 di[(2E) 3-{4-[(3-methoxy tetrafluoroethoxy)benzoyl)oxy]phenyl}prop-2-enoyl]propanediol
2-(2,4-Diaminophenyl)-1,3 di[(2E) 3-{4-[(3-methoxy 4-(4,4,4-trifluorobutanoyl)oxy)benzoyl]oxy}phenyl}prop-2-enoyl]propanediol
2-(2,4-Diaminophenyl)-1,3 di[(2E) 3-{4-[(4-trifluoromethoxybenzoyl)oxy]-3-methoxyphenyl}prop-2-enoyl]propanediol
2-(2,4-Diaminophenyl)-1,3 di[(2E) 3-{4-[(4-(2,2,2-trifluoroethoxy)benzoyl)oxy]-3-methoxyphenyl}prop-2-enoyl]propanediol
2-(2,4-Diaminophenyl)-1,3 di[(2E) 3-{4-[(4-(4,4,4-trifluorobutoxy)benzoyl)oxy]-3-methoxy phenyl}prop-2-enoyl]propanediol
2-(2,4-Diaminophenyl)-1,3 di[(2E) 3-{4-[(4-(5,5,5-trifluoropentyloxy)benzoyl)oxy]-3-methoxyphenyl}prop-2-enoyl]propanediol
2-(2,4-Diaminophenyl)-1,3 di[(2E) 3-{4-[(4-(4,4,5,5,5-pentafluoropentoxy)benzoyl)oxy]-3-methoxyphenyl}prop-2-enoyl]propanediol
2-(2,4-Diaminophenyl)-1,3 di[(2E) 3-{4-[(4-(1,1,2,2-tetrafluoroethoxy)benzoyl)oxy]-3-methoxyphenyl}prop-2-enoyl]propanediol
2-(2,4-Diaminophenyl)-1,3 di[(2E) 3-{4-[(4-(4,4,4-trifluorobutanoyl)oxy)benzoyl]oxy}-3-methoxyphenyl}prop-2-enoyl]propanediol
2-(2,4-Diaminophenyl)-1,3 di[(2E) 3-{4-[(4-trifluoromethoxyphenoxy)carbonyl]phenyl}prop-2-enoyl]propanediol
2-(2,4-Diaminophenyl)-1,3 di[(2E) 3-{4-[(4-(2,2,2-trifluoroethoxy)phenoxy)carbonyl]phenyl}prop-2-enoyl]propanediol
2-(2,4-Diaminophenyl)-1,3 di[(2E) 3-{4-[(4-(4,4,4-trifluorobutoxy)phenoxy)carbonyl]phenyl}prop-2-enoyl]propanediol
2-(2,4-Diaminophenyl)-1,3 di[(2E) 3-{4-[(4-(5,5,5-trifluoropentyloxy)phenoxy)carbonyl]phenyl}prop-2-enoyl]propanediol
2-(2,4-Diaminophenyl)-1,3 di[(2E) 3-{4-[(4-(4,4,5,5,5-pentafluoropentoxy)phenoxy)carbonyl]phenyl}prop-2-enoyl]propanediol
2-(2,4-Diaminophenyl)-1,3 di[(2E) 3-{4-[(4-(1,1,2,2-tetrafluoroethoxy)phenoxy)carbonyl]phenyl}prop-2-enoyl]propanediol
2-(3,5-Diaminophenyl)-1,3 di[(2E) 3-{4-[(4-trifluoromethoxybenzoyl)oxy]phenyl}prop-2-enoyl]propanediol
2-(3,5-Diaminophenyl)-1,3 di[(2E) 3-{4-[(4-(2,2,2-trifluoroethoxy)benzoyl)oxy]phenyl}prop-2-enoyl]propanediol
2-(3,5-Diaminophenyl)-1,3 di[(2E) 3-{4-[(4-(4,4,4-trifluorobutoxy)benzoyl)oxy]phenyl}prop-2-enoyl]propanediol
2-(3,5-Diaminophenyl)-1,3 di[(2E) 3-{4-[(4-(5,5,5-trifluoropentyloxy)benzoyl)oxy]phenyl}prop-2-enoyl]propanediol
2-(3,5-Diaminophenyl)-1,3 di[(2E) 3-{4-[(4-(4,4,5,5,5-pentafluoropentoxy)benzoyl)oxy]phenyl}prop-2-enoyl]propanediol
2-(3,5-Diaminophenyl)-1,3 di[(2E) 3-{4-[(4-(1,1,2,2-tetrafluoroethoxy)benzoyl)oxy]phenyl}prop-2-enoyl]propanediol
2-(3,5-Diaminophenyl)-1,3 di[(2E) 3-{4-[(4-(4,4,4-trifluorobutanoyl)oxy)benzoyl]oxy}phenyl}prop-2-enoyl]propanediol
2-(3,5-Diaminophenyl)-1,3 di[(2E) 3-{4-[(3-methoxy-4-trifluoromethoxybenzoyl)oxy]phenyl}prop-2-enoyl]propanediol
2-(3,5-Diaminophenyl)-1,3 di[(2E) 3-{4-[(3-methoxy 4-(2,2,2-trifluoroethoxy)benzoyl)oxy]phenyl}prop-2-enoyl]propanediol
2-(3,5-Diaminophenyl)-1,3 di[(2E) 3-{4-[(3-methoxy 4-(4,4,4-trifluorobutoxy)benzoyl)oxy]phenyl}prop-2-enoyl]propanediol
2-(3,5-Diaminophenyl)-1,3 di[(2E) 3-{4-[(3-methoxy 4-(5,5,5-trifluoropentyloxy)benzoyl)oxy]phenyl}prop-2-enoyl]propanediol
2-(3,5-Diaminophenyl)-1,3 di[(2E) 3-{4-[(3-methoxy 4-(4,4,5,5,5-pentafluoropentoxy)benzoyl)oxy]phenyl}prop-2-enoyl]propanediol
2-(3,5-Diaminophenyl)-1,3 di[(2E) 3-{4-[(3-methoxy 4-(1,1,2,2-tetrafluoroethoxy)benzoyl)oxy]phenyl}prop-2-enoyl]propanediol
2-(3,5-Diaminophenyl)-1,3 di[(2E) 3-{4-[(3-methoxy 4-(4,4,4-trifluorobutanoyl)oxy)benzoyl]oxy}phenyl}prop-2-enoyl]propanediol
2-(3,5-Diaminophenyl)-1,3 di[(2E) 3-{4-[(4-trifluoromethoxybenzoyl)oxy]-3-methoxyphenyl}prop-2-enoyl]propanediol
2-(3,5-Diaminophenyl)-1,3 di[(2E) 3-{4-[(4-(2,2,2-trifluoroethoxy)benzoyl)oxy]-3-methoxyphenyl}prop-2-enoyl]propanediol
2-(3,5-Diaminophenyl)-1,3 di[(2E) 3-{4-[(4-(4,4,4-trifluorobutoxy)benzoyl)oxy]-3-methoxy phenyl}prop-2-enoyl]propanediol
2-(3,5-Diaminophenyl)-1,3 di[(2E) 3-{4-[(4-(5,5,5-trifluoropentyloxy)benzoyl)oxy]-3-methoxyphenyl}prop-2-enoyl]propanediol
2-(3,5-Diaminophenyl)-1,3 di[(2E) 3-{4-[(4-(4,4,5,5,5-pentafluoropentoxy)benzoyl)oxy]-3-methoxyphenyl}prop-2-enoyl]propanediol
2-(3,5-Diaminophenyl)-1,3 di[(2E) 3-{4-[(4-(1,1,2,2-tetrafluoroethoxy)benzoyl)oxy]-3-methoxyphenyl}prop-2-enoyl]propanediol
2-(3,5-Diaminophenyl)-1,3 di[(2E) 3-{4-[(4-(4,4,4-trifluorobutanoyl)oxy)benzoyl]oxy}-3-methoxyphenyl}prop-2-enoyl]propanediol
2-(3,5-Diaminophenyl)-1,3 di[(2E) 3-{4-[(4-trifluoromethoxyphenoxy)carbonyl]phenyl}prop-2-enoyl]propanediol
2-(3,5-Diaminophenyl)-1,3 di[(2E) 3-{4-[(4-(2,2,2-trifluoroethoxy)phenoxy)carbonyl]phenyl}prop-2-enoyl]propanediol
2-(3,5-Diaminophenyl)-1,3 di[(2E) 3-{4-[(4-(4,4,4-trifluorobutoxy)phenoxy)carbonyl]phenyl}prop-2-enoyl]propanediol
2-(3,5-Diaminophenyl)-1,3 di[(2E) 3-{4-[(4-(5,5,5-trifluoropentyloxy)phenoxy)carbonyl]phenyl}prop-2-enoyl]propanediol
2-(3,5-Diaminophenyl)-1,3 di[(2E) 3-{4-[(4-(4,4,5,5,5-pentafluoropentoxy)phenoxy)carbonyl]phenyl}prop-2-enoyl]propanediol
2-(3,5-Diaminophenyl)-1,3 di[(2E) 3-{4-[(4-(1,1,2,2-tetrafluoroethoxy)phenoxy)carbonyl]phenyl}prop-2-enoyl]propanediol

### EXAMPLE 9

### Polymersiation Step A

### Formation of the Polyamic Acid

2.25 g (11.47 mmol) of 1,2,3,4-cyclobutantetracarboxylic acid dianhydride is added to a solution of 8.030 g (12.77 mmol) of 6-{[((2E)-3-{4-[(4-(4,4,4-trifluorobutoxy)benzoyl)oxy]phenyl}prop-2-enoyl)oxy]}hexyl 3,5-Diaminobenzoate in 56.0 ml of tetrahydrofuran. Stirring is then carried out at 0° C. for 2 hours. Then another 0.255 g (1.30 mmol) of 1,2,3,4-cyclobutantetracarboxylic acid dianhydride are added. The mixture is subsequently allowed to react for 21 hours at room temperature. The polymer mixture is diluted with 56 ml THF, precipitated into 2000 ml diethyl ether and collected by filtration. The polymer is reprecipitated form THF (160 ml) into 3500 ml water to yield, after drying at room temperature under vacuum, 9.42 g of Polyamic Acid 1 in the from of a white powder; [η]=0.50 dL/g

Analogous to EXAMPLE 9 the following diamines are used for the preparation of Polyamic Acid with 1,2,3,4-cyclobutantetracarboxylic acid dianhydride

### 6-{[((2E)-3-{4-[(4-(3,3,3-trifluoropropoxy)benzoyl)oxy]phenyl}prop-2-enoyl)oxy]}hexyl 3,5-Diaminobenzoate

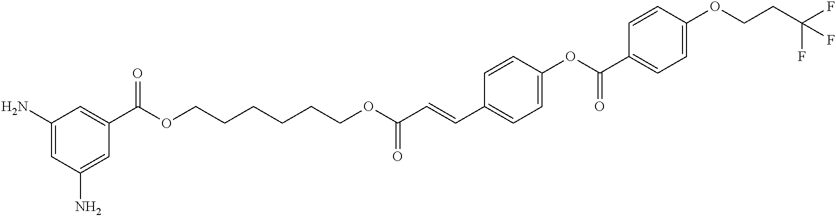

yield Polyamic acid 2 as white powder; [η]=0.24 dL/g

### 6-{[((2E)-3-{4-[(3-methoxy 4-(4,4,4-trifluorobutoxy)benzoyl)oxy]phenyl}prop-2-enoyl)oxy]}hexyll 3,5-Diaminobenzoate

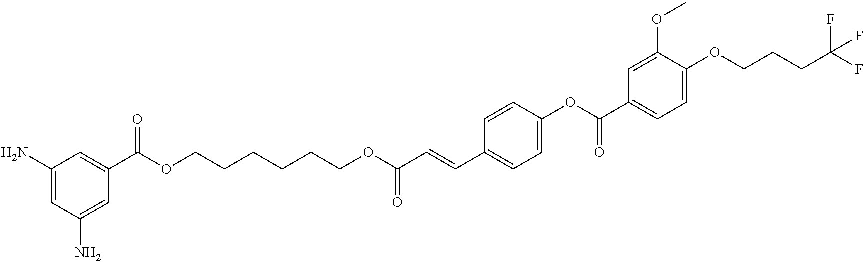

yield Polyamic acid 3 as white powder; [η]=0.25 dL/g.

### 8-{[((2E)-3-{4-[(4-(4,4,4-trifluorobutoxy)benzoyl)oxy]phenyl}prop-2-enoyl)oxy]}octyl 3,5-Diaminobenzoate

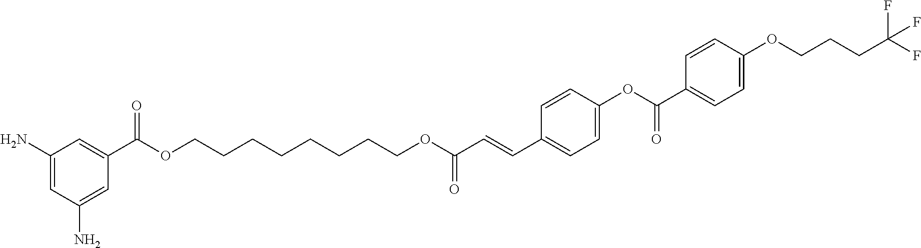

yield Polyamic acid 4 as white powder; [η]=1.09 dL/g.

### 4-{[((2E)-3-{4-[(4-(4,4,4-trifluorobutoxy)benzoyl)oxy]phenyl}prop-2-enoyl)oxy]}butyl 3,5-Diaminobenzoate

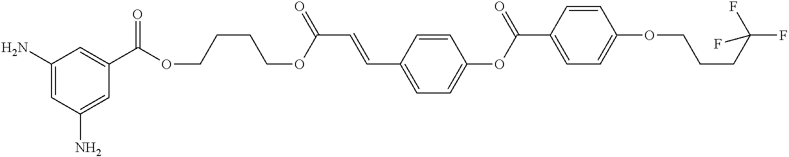

yield Polyamic acid 5 as white powder; [η]=0.21 dL/g.

### 2-[2-{[((2E)-3-{4-[(4-(4,4,4-trifluorobutoxy)benzoyl)oxy]phenyl}prop-2-enoyl)oxy]}ethoxy]ethyl 3,5-Diaminobenzoate

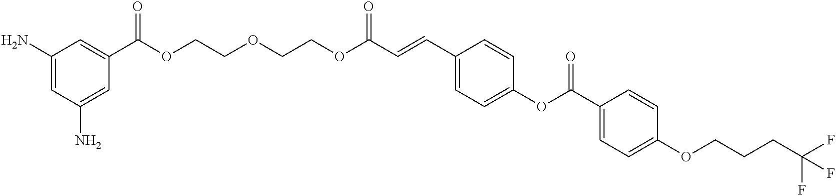

yield Polyamic acid 6 as white powder; [η]=0.87 dL/g.

### 2-{[((2E)-3-{4-[(4-(4,4,4-trifluorobutoxy)benzoyl)oxy]phenyl}prop-2-enoyl)oxy]}ethyl 3,5-Diaminobenzoate

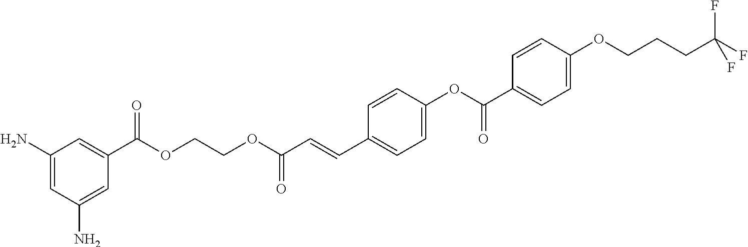

yield Polyamic acid 7 as white powder; [η]=0.48 dL/g.

### 3-{[((2E)-3-{4-[(4-(4,4,4-trifluorobutoxy)benzoyl)oxy]phenyl}prop-2-enoyl)oxy]}propyl 3,5-Diaminobenzoate yield

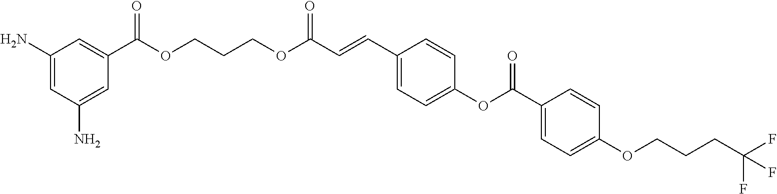

Polyamic acid 8 as white powder; [η]=0.63 dL/g.

### 6-{[((2E)-3-{4-[(4-(4,4,5,5,5-pentafluoropentoxy)benzoyl)oxy]phenyl}prop-2-enoyl)oxy]}hexyl 3,5-Diaminobenzoate

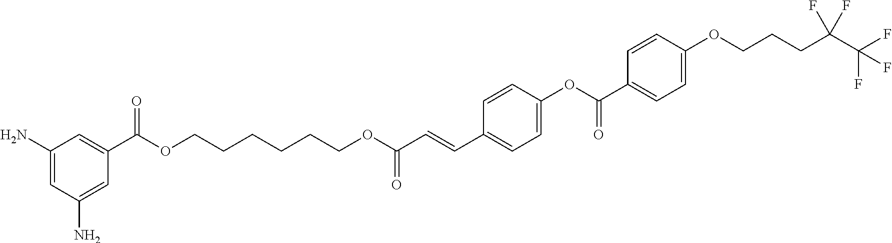

yield Polyamic acid 9 as white powder; [η]=0.26 dL/g.

### 6-{[((2E)-3-{4-[(4-trifluoromethoxybenzoyl)oxy]phenyl}prop-2-enoyl)oxy]}hexyl 3,5-Diaminobenzoate

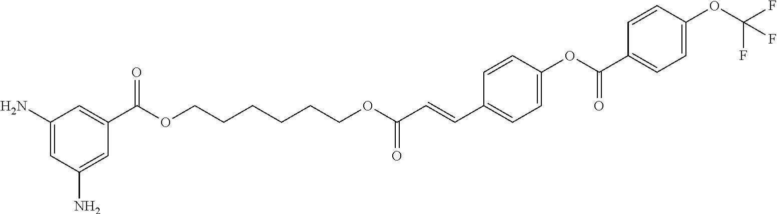

yield Polyamic acid 10 as white powder; [η]=0.71 dL/g

### 6-{[((2E)-3-{4-[(4-trifluoromethylbenzoyl)oxy]phenyl}prop-2-enoyl)oxy]}hexyl 3,5-Diaminobenzoate

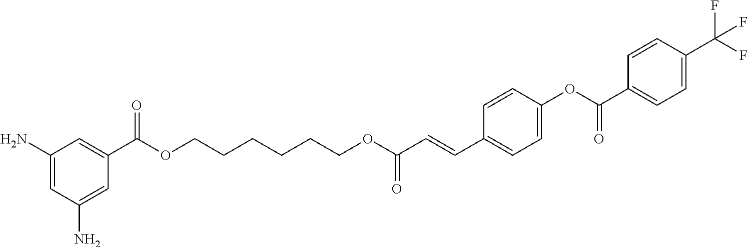

yield Polyamic acid 11 as white powder; [η]=1.21 dL/g

### 6-{[((2E)-3-{4-[(4-(2,2,3,3-tetrafluoropropoxy)benzoyl)oxy]phenyl}prop-2-enoyl)oxy]}hexyl 3,5-Diaminobenzoate

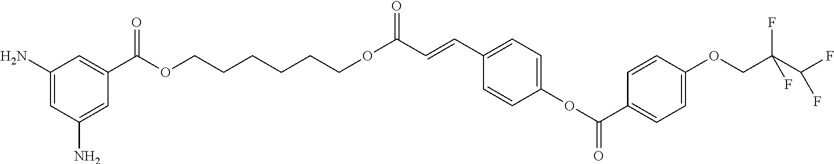

yield Polyamic acid 12 as white powder; [η]=0.48 dL/g

### 6-{[((2E)-3-{4-[(4-(2,2,3,3-tetrafluoroethoxy)benzoyl)oxy]phenyl}prop-2-enoyl)oxy]}hexyl 3,5-Diaminobenzoate

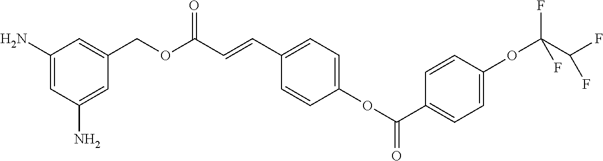

yield Polyamic acid 13 as white powder; [η]=0.48 dL/g

### 3,5-Diaminobenzyl (2E) 3-{4-[(4-(4,4,5,5,5-pentafluoropentyloxy)benzoyl)oxy]phenyl}acrylate

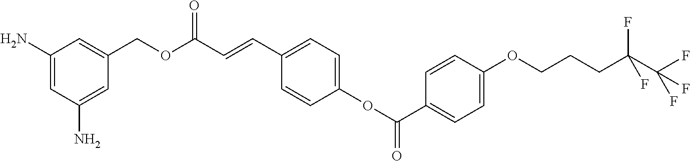

yield Polyamic acid 14 as white powder; [η]=0.59 dL/g

### 3,5-Diaminobenzyl (2E) 3-{4-[(4-(4,4,5,5,6,6,6-heptafluorohexyloxy)benzoyl)oxy]phenyl}acrylate

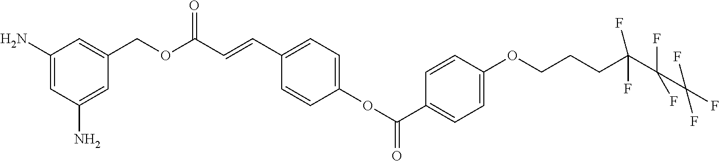

yield Polyamic acid 15 as white powder; [η]=0.20 dL/g

### 3,5-Diaminobenzyl (2E) 3-{4-[(4-(5,5,5-trifluorobutoxy)benzoyl)oxy]phenyl}acrylate

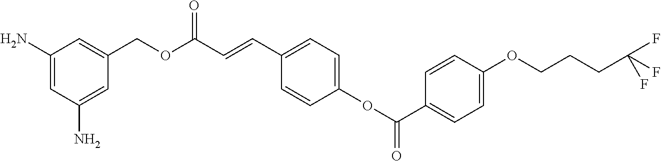

yield Polyamic acid 16 as white powder; [η]=0.38 dL/g

### 2-(2,4-Diaminophenyl)ethyl (2E) 3-{4-[(4-(4,4,4-trifluorobutoxy)benzoyl)oxy]phenyl}acrylate

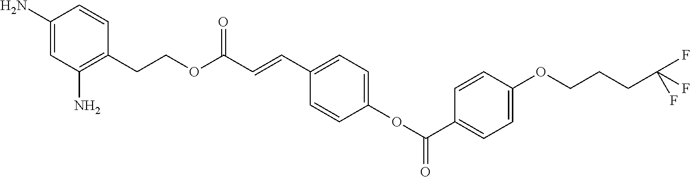

yield Polyamic acid 17 as white powder; [η]=0.50 dL/g

### 2-(2,4-Diaminophenyl)ethyl (2E) 3-{4-[(4-(4,4,5,5,5-pentafluoropentyloxy)benzoyl)oxy]phenyl}acrylate

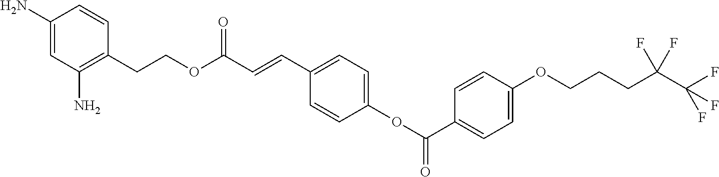

yield Polyamic acid 18 as white powder; [η]=0.27 dL/g

### 2-(2,4-Diaminophenyl)ethyl (2E) 3-{4-[(4-(4,4,5,5,6,6,6-heptafluorohexyloxy)benzoyl)oxy]phenyl}acrylate

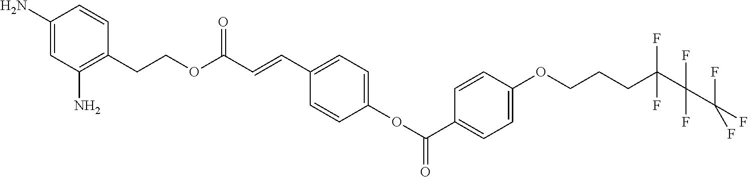

yield Polyamic acid 19 as white powder; [η]=0.19 dL/g

### 2-(2,4-Diaminophenyl)ethyl (2E) 3-{4-[(4-(2,2,3,3-tetrafluoroethoxy)benzoyl)oxy]phenyl}acrylate

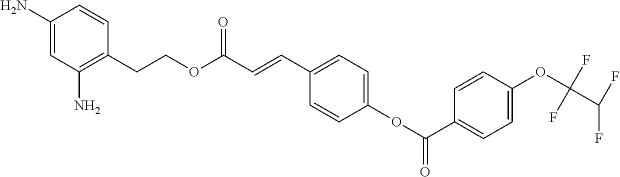

yield Polyamic acid 20 as white powder; [η]=0.28 dL/g

### 2,2-bis(4-aminobenzyl)-1,3 di[(2E)-3-{4-[(4-(4,4,4-trifluorobutoxy)benzoyl)oxy]phenyl}prop-2-enoyl]propanediol

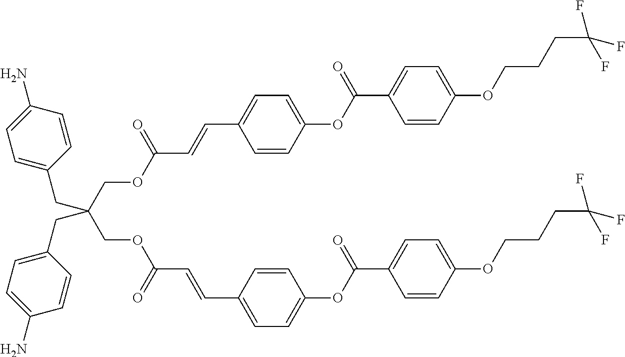

yield Polyamic acid 21 as white powder; [η]=0.54 dL/g

### 2-(2,4-Dinitrophenyl)-1,3 di[(2E)-3-{4-[(4-(4,4,4-trifluorobutoxy)benzoyl)oxy]phenyl}prop-2-enoyl]propanediol

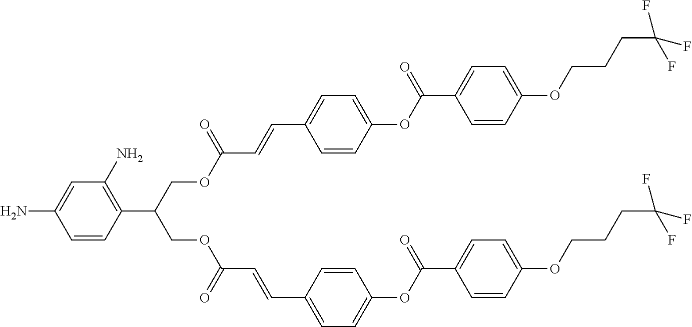

yield Polyamic acid 22 as white powder; [n]=0.17 dL/g

### 2-(2,4-Dinitrophenyl)-1,3 di[(2E)-3-{4-[(4-(4,4,5,5,5-pentafluoropentyloxy)benzoyl)oxy]phenyl}prop-2-enoyl]propanediol

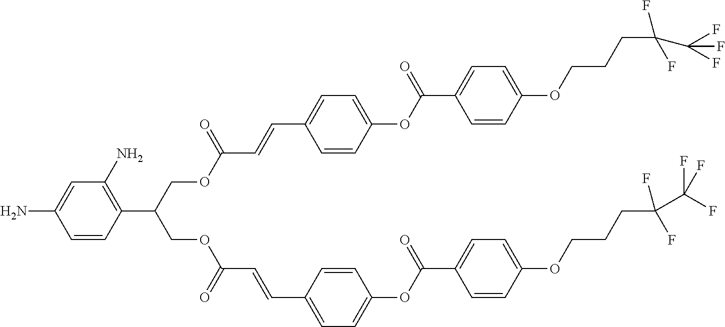

yield Polyamic acid 23 as white powder; [η]=0.16 dL/g

### 2,2′-bis[(2E)-3-{4-[(4-(4,4,5,5,5-pentafluoropentyloxy)benzoyl)oxy]phenyl}prop-2-enoyl]methyl 4,4′-Diamino 1,1′-biphenyl

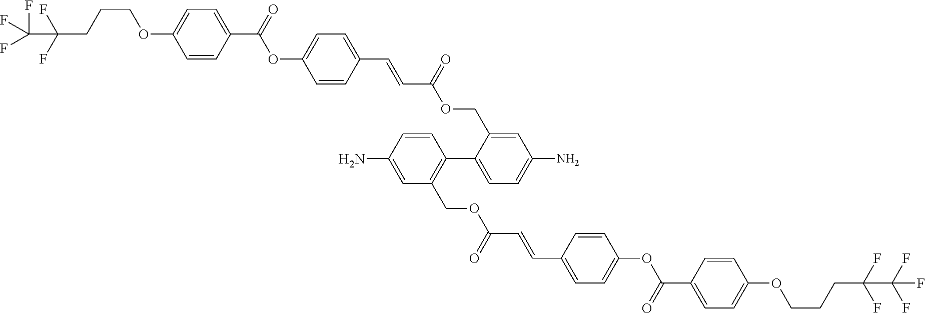

yield Polyamic acid 24 as white powder; [η]=0.55 dL/g

### EXAMPLE 10

Analogous to EXAMPLE 9 the following diamines are used for the preparation of Polyamic Acid with 2,3,5-tricarboxycyclopentylacetic acid dianhydride

### 3,5-Diaminobenzyl (2E) 3-{4-[(4-(5,5,5-trifluorobutoxy)benzoyl)oxy]phenyl}acrylate

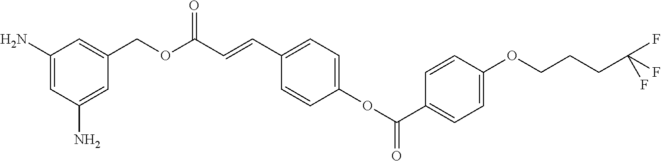

yield Polyamic acid 25 as white powder; [η]=0.40 dL/g

### 2,2-bis(4-aminobenzyl)-1,3 di[(2E)-3-{4-[(4-(4,4,4-trifluorobutoxy)benzoyl)oxy]phenyl}prop-2-enoyl]propanediol

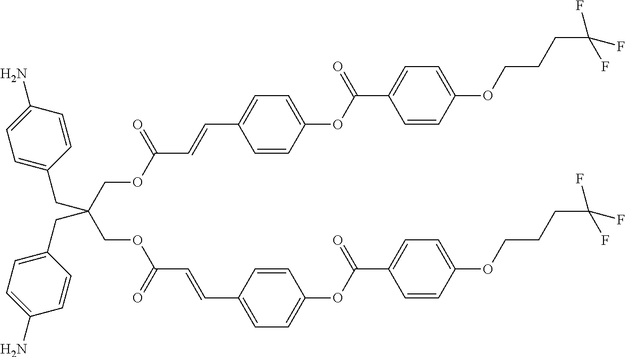

yield Polyamic acid 26 as white powder; [η]=0.47 dL/g

### 2-(2,4-Diaminophenyl)ethyl (2E) 3-{4-[(4-(4,4,4-trifluorobutoxy)benzoyl)oxy]phenyl}acrylate

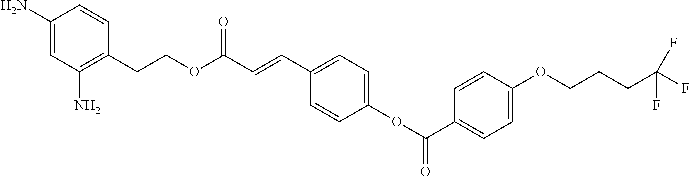

yield Polyamic acid 27 as white powder; [η]=0.23 dL/g

### 2-(2,4-Diaminophenyl)ethyl (2E) 3-{4-[(4-(4,4,5,5,5-pentafluoropentyloxy)benzoyl)oxy]phenyl}acrylate

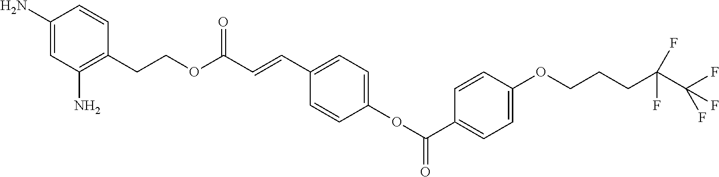

yield Polyamic acid 28 as white powder; [η]=0.14 dL/g

### 3,5-Diaminobenzyl (2E) 3-{4-[(4-(5,5,5-trifluorobutoxy)benzoyl)oxy]phenyl}acrylate

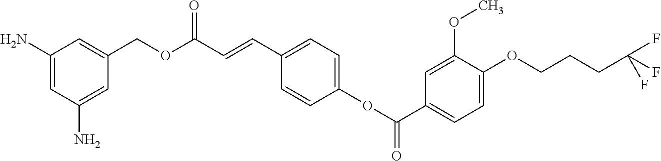

yield Polyamic acid 29 as white powder; [η]=0.45 dL/g

### 2,2′-bis[(2E)-3-{4-[(4-(4,4,5,5,5-pentafluoropentyloxy)benzoyl)oxy]phenyl}prop-2-enoyl]methyl 4,4′-Diamino 1,1′-biphenyl

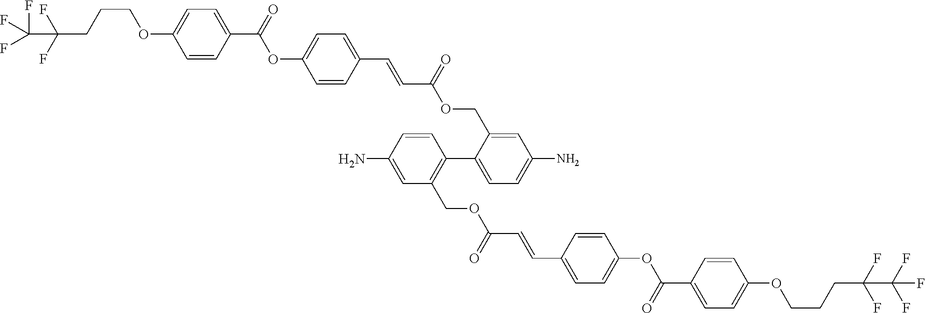

yield Polyamic acid 30 as white powder; [η]=0.30 dL/g

### 2,2′-bis[(2E)-3-{4-[(4-(4,4,4-trifluorobutoxy)benzoyl)oxy]phenyl}prop-2-enoyl]methyl 4,4′-Diamino 1,1′-biphenyl

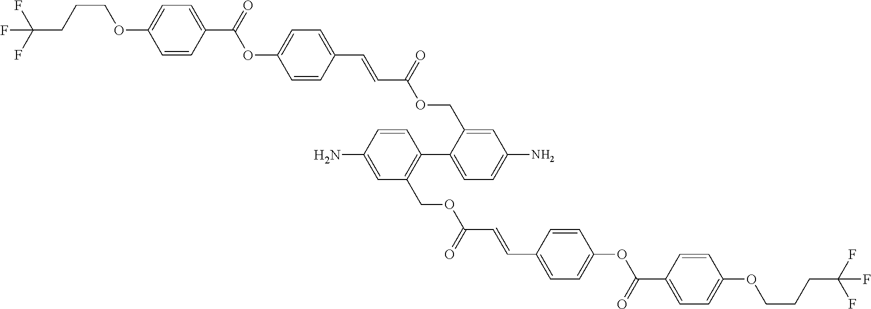

yield Polyamic acid 31 as white powder; [η]=0.17 dL/g

### 2-(2,4-Dinitrophenyl)-1,3 di[(2E)-3-{4-[(4-(4,4,4-trifluorobutoxy)benzoyl)oxy]phenyl}prop-2-enoyl]propanediol

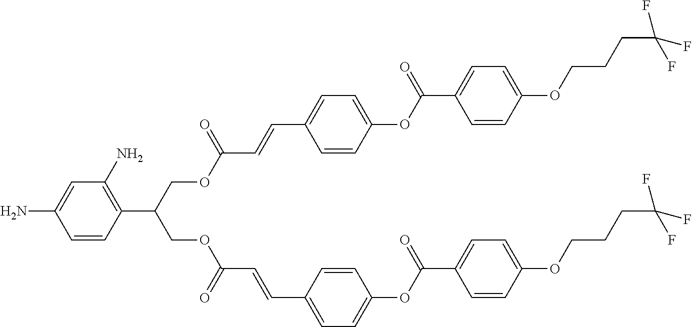

### 2-(2,4-Dinitrophenyl)-1,3 di[(2E)-3-{4-[(4-(4,4,5,5,5-pentafluoropentyloxy)benzoyl)oxy]phenyl}prop-2-enoyl]propanediol

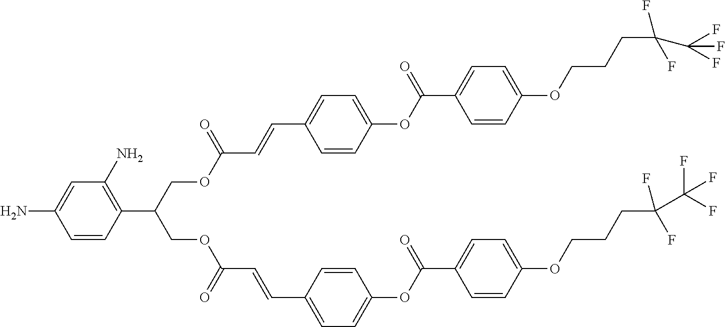

### 3,5-Diaminobenzyl (2E) 3-{4-[(4-(4,4,5,5,5-pentafluoropentyloxy)benzoyl)oxy]phenyl}acrylate

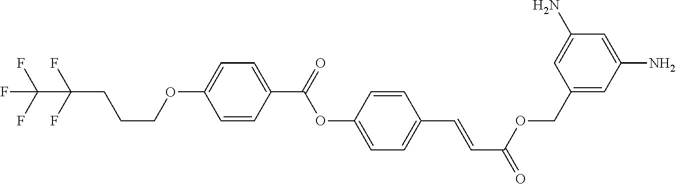

yield Polyamic acid 50 as white powder; [η]=0.39 dL/g

### 6-{[((2E)-3-{4-[(4-(4,4,4-trifluorobutoxy)benzoyl)oxy]phenyl}prop-2-enoyl)oxy]}hexyl 3,5-diaminobenzoate

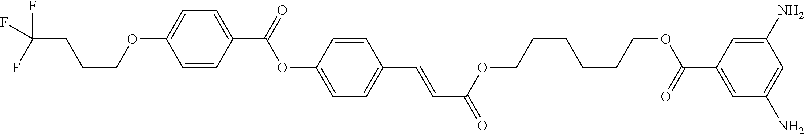

yield Polyamic acid 51 as white powder; [η]=0.43 dL/g

### EXAMPLE 11

Analogous to EXAMPLE 9 the following tetracarboxylic acid dianhydride are used for the preparation of Polyamic Acid with of 6-{[((2E)-3-{4-[(4-(4,4,4-trifluorobutoxy)benzoyl)oxy]phenyl}prop-2-enoyl)oxy]}hexyl 3,5-Diaminobenzoate.

4-(2,5-dioxotetrahydrofuran-3-yl)tetrahydronaphthalene-1,2-dicarboxylicacid dianhydrid e Diaminobenzoate yield Polyamic acid 32 as white powder; [η]=0.15 dL/g.

bicyclo[2.2.2]oct-7-ene-2,3,5,6-tetracarboxylic acid dianhydride yield Polyamic acid 33 as white powder; [η]=0.11 dL/g

2,3,5-tricarboxycyclopentylacetic acid dianhydride yield Polyamic acid 34 as white powder; [η]=0.43 dL/g

5-(2,5-dioxotetrahydrofuran-3-yl)-3-methyl-3-cyclohexene-1,2-dicarboxylic-acid dianhydride yield Polyamic acid 35 as white powder; [η]=0.16 dL/g

4,4′-(hexafluoroisopropylidene)diphthalic acid dianhydride yield Polyamic acid 36 as white powder; [η]=0.51 dL/g

### EXAMPLE 12

Analogous to EXAMPLE 9 the following tetracarboxylic acid dianhydride mixture are used for the preparation of Polyamic Acid with of 6-{[((2E)-3-{4-[(4-(4,4,4-trifluorobutoxy)benzoyl)oxy]phenyl}prop-2-enoyl)oxy]}hexyl 3,5-Diaminobenzoate.

A mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 25:75 (mole ratio) yield Polyamic acid 37 as white powder; [η]=0.16 dL/g

A mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 1:1 (mole ratio) yield Polyamic acid 38 as white powder; [η]=0.20 dL/g

A mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 75:25 (mole ratio) yield Polyamic acid 39 as white powder; [η]=0.20 dL/g

A mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 90:10 (mole ratio) yield Polyamic acid 40 as white powder; [η]=0.17 dL/g

A mixture of 1,2,3,4-cyclobutantetracarboxylic acid dianhydride and 5-(2,5-dioxotetrahydrofuran-3-yl)-3-methyl-3-cyclohexene-1,2-dicarboxylic-acid dianhydride 25:75 (mole ratio) yield Polyamic acid 41 as white powder; [η]=0.16 dL/g

A mixture of 1,2,3,4-cyclobutantetracarboxylic acid dianhydride and 5-(2,5-dioxotetrahydrofuran-3-yl)-3-methyl-3-cyclohexene-1,2-dicarboxylic-acid dianhydride 1:1 (mole ratio) yield Polyamic acid 42 as white powder; [η]=0.16 dL/g

A mixture of 1,2,3,4-cyclobutantetracarboxylic acid dianhydride and 5-(2,5-dioxotetrahydrofuran-3-yl)-3-methyl-3-cyclohexene-1,2-dicarboxylic-acid dianhydride 75:25 (mole ratio) yield Polyamic acid 43 as white powder; [η]=0.16 dL/g

### EXAMPLE 13

Analogous to EXAMPLE 9 a mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 75:25 (mole ratio) and 3,5-Diaminobenzyl (2E) 3-{4-[(4-(4,4,5,5,5-pentafluoropentyloxy)benzoyl)oxy]phenyl}acrylate were used for the preparation to yield Polyamic acid 44 as white powder; [η]=0.17 dL/g

### EXAMPLE 14

Analogous to EXAMPLE 9 a mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 75:25 (mole ratio) and 3,5-Diaminobenzyl (2E) 3-{4-[(4-(4,4,4-trifluorobutoxy)benzoyl)oxy]phenyl}acrylate were used for the preparation to yield Polyamic acid 45 as white powder; [η]=0.24 dL/g

### EXAMPLE 15

Analogous to EXAMPLE 9 a mixture of 1,2,3,4-cyclobutantetracarboxylic acid dianhydride and 4-(2,5-dioxotetrahydrofuran-3-yl)tetrahydronaphthalene-1,2-dicarboxylic acid dianhydride 75:25 (mole ratio) and 2-(2,4-Diaminophenyl)ethyl (2E) 3-{4-[(4-(4,4,5,5,5-pentafluoropentyloxy)benzoyl)oxy]phenyl}acrylate are used for the preparation to yield Polyamic acid 46 as white powder; [η]=0.11 dL/g

### EXAMPLE 16

Analogous to EXAMPLE 9 a mixture of 2,2-bis(4-aminobenzyl)-1,3 di[(2E)-3-{4-[(4-(4,4,4-trifluorobutoxy)benzoyl)oxy]phenyl}prop-2-enoyl]propanediol and 6-{[((2E)-3-{4-[(4-(4,4,4-trifluorobutoxy)benzoyl)oxy]phenyl}prop-2-enoyl)oxy]}hexyl 3,5-Diaminobenzoate 1:1 (mole ratio) and 1,2,3,4-cyclobutantetracarboxylic acid dianhydride are used for the preparation to yield Polyamic acid 47 as white powder; [η]=0.98 dL/g

### EXAMPLE 17

Analogous to EXAMPLE 9 a mixture of 2,2-bis(4-aminobenzyl)-1,3 di[(2E)-3-{4-[(4-(4,4,4-trifluorobutoxy)benzoyl)oxy]phenyl}prop-2-enoyl]propanediol and 4,4′-Diaminodiphenylmethane 80:20 (mole ratio) and 1,2,3,4-cyclobutantetracarboxylic acid dianhydride are used for the preparation to yield Polyamic acid 48 as white powder; [η]=1.00 dL/g

### EXAMPLE 18

### Polymerisation Step B

### Formation of the Polyimide

0.50 g of Polyamic Acid No. 1 obtained in above EXAMPLE 9 are dissolved in 3 ml of 1-methyl-2-pyrrolidon (NMP). Thereto are added 0.28 g (3.57 mmol, 4 equivalent) of pyridine and 364 mg (3.57 mmol, 4 equivalent) acetic acid anhydride, and the dehydration and ring closure is carried out at 80° C. for 2 h. The polymer mixture is diluted with 1.5 ml NMP, precipitated into 100 ml diethyl ether and collected by filtration. The polymer is reprecipitated from THF (10 ml) into 200 ml water to yield, after drying at room temperature under vacuum, 0.55 g Polyimide No 1; [η]=0.50 dL/g, Imidization degree ID=100%

Analogous to the polymerization step of EXAMPLE 18 the following polyamic acids are used for the preparation of partially imidizated polyimide. The imidization degree is adjusted with the ratio of acetic acid anhydride and pyridine.

Polyamic acid 1 with 1.2 equivalent acetic acid anhydride and pyridine yield Polyimide 1 as white powder; [η]=0.23 dL/g, ID=40%.

Polyamic acid 1 with 0.8 equivalent acetic acid anhydride and pyridine yield Polyimide 1 as white powder; [η]=0.26 dL/g, ID=30%.

Polyamic acid 1 with 0.4 equivalent acetic acid anhydride and pyridine yield Polyimide 1 as white powder; [η]=0.27 dL/g, ID=14%.

Polyamic acid 2 yield Polyimide 2 as white powder; [η]=0.24 dL/g, ID=100%

Polyamic acid 5 yield Polyimide 5 as white powder; [η]=0.36 dL/g, ID=100%

Polyamic acid 13 yield Polyimide 14 as white powder; [η]=0.88 dL/g, 10=100%

Polyamic acid 14 yield Polyimide 13 as white powder; [η]=0.48 dL/g, 10=100%

Polyamic acid 15 yield Polyimide 15 as white powder; [η]=0.20 dL/g, ID=100%

Polyamic acid 16 yield Polyimide 16 as white powder; [η]=0.27 dL/g, 10=100%

Polyamic acid 17 yield Polyimide 17 as white powder; [η]=0.29 dL/g, 10=100%

Polyamic acid 18 yield Polyimide 18 as white powder; [η]=0.28 dL/g, 10=100%

Polyamic acid 19 yield Polyimide 19 as white powder; [η]=0.19 dL/g, 10=100%

Polyamic acid 20 yield Polyimide 20 as white powder; [η]=0.28 dL/g, 10=100%

Polyamic acid 21 yield Polyimide 21 as white powder; [η]=0.63 dL/g, 10=100%

Polyamic acid 25 yield Polyimide 25 as white powder; [η]=0.43 dL/g, 10=100%

Polyamic acid 27 yield Polyimide 27 as white powder; [η]=0.20 dL/g, 10=100%

Polyamic acid 28 yield Polyimide 28 as white powder; [η]=0.14 dL/g, 10=60%

Polyamic acid 28 with 1.0 equivalent acetic acid anhydride and pyridine yield Polyimide 28 as white powder; [η]=0.23 dL/g, 10=25%.

Polyamic acid 34 yield Polyimide 34 as white powder; [η]=0.40 dL/g, ID=100%

Polyamic acid 39yield Polyimide 39 as white powder; [η]=0.21 dL/g, 10=100%

Polyamic acid 44 yield Polyimide 44 as white powder; [η]=0.14 dL/g, 10=100%

Polyamic acid 45 yield Polyimide 45 as white powder; [η]=0.12 dL/g, 10=100%

Polyamic acid 50 yield Polyimide 50 as white powder; [η]=0.39 dL/g, 10=100%

Polyamic acid 51 yield Polyimide 51 as white powder; [η]=0.43 dL/g, 10=100%

### EXAMPLE 19

### Preparation of an Orientation Layer for Vertical Alignment with Non-Polarized UV Light

A 4% solution of LPP (see molecular structure on FIG. 1) in a solvent mixture of N-Methyl-2-Pyrrolidone (NMP) and Butylglycol (BC) in a ratio of 1:9 by weight was prepared. This LPP solution was filtered over a 2 μm Teflon filter and applied to two indium tin oxyde (ITO) coated rectangular glass plates by spin coating at 1350 rpm for 30 seconds. The resulting films were then pre-dried for 5 minutes at 130° C. and further post-baked for 40 minutes at 200° C.

Both ITO covered glass plates were irradiated with non-polarised UV light at a dose of 48 mJ/cm². The direction of incidence of the light being inclined by 10° relative to the plate normal and the incidence plane was parallel to the short side of the substrate. The two irradiated plates were used to build a cell of 20 μm spacing in an anti-parallel manner such that the irradiated surfaces were facing each other. The cell was then capillary filled with liquid crystal mixture MLC6610 from Merck in the isotropic phase at 105° C. The cell was then gradually cooled down at a rate of 0.1° C./min from T=105° C. to T=85° C. and at a rate of 2 C/min from T=85° C. to room temperature. When arranged between crossed polarisers, the cell appeared uniformly black for every angle between the short edge of the cell and the polariser transmission axis, as long as viewed from the vertical. In conclusion, the liquid crystal mixture was aligned homeotropically.

When the short edge of the cell was set at 45° to the polariser axis and an AC voltage of 7V and 90 Hz was applied, the liquid crystals switched and caused the cell to appear green (high order birefringence). No defects or tilt domains were observed. Brightness and colour of the switched cell changed asymmetrically when viewed from opposite, but equal oblique angles along a plane parallel to the short edge of the cell. Contrary, no asymmetry was found when viewed obliquely from opposite angles within a plane parallel to the long edge of the cell. When the switched cell with its short edge was aligned parallel or perpendicular to one of the polariser transmission axes the cell appeared dark again. From above observations we concluded that LC alignment capability was induced in the thin film on the substrate due to irradiation with slantwise incident non-polarized light. The azimuthal alignment direction was parallel to the plane of incidence of the non-polarized uv-light.

From tilt angle evaluation by means of the crystal rotation method a tilt angle value of 89.2° with respect to the substrate surface was obtained. The direction of the LC molecules was in between the surface normal and the direction of the incident light.

### EXAMPLE 20

The following experiments were carried out in order to characterize the contamination in the “uncoated areas” of the orientation layer as well as in any part of the device, display or equipment which might be contaminated, due to the thermal instability of the alignment material by means of surface energy measurements. Actually, a change of surface energy would signify the contamination of the uncoated areas through adsorption/migration of the alignment material e.g. volatile fragments of the alignment material, which could have detrimental effects on subsequent coatings. Thus, the wetting and/or adhesion properties of coatings or liquids subsequently applied on these “uncoated areas” would be changed which would lead to defects (e.g. adhesion failure). It is well known that wetting and good adhesion are favoured when the substrate's critical surface tension is high and the surface tension of the coating/adhesive is low: hence, failures or defects might arise if the difference in the surface tension between the coating formulation to be applied and the surface energy of the “uncoated areas” are not respecting this basic rule. Modification, in particular decrease of the surface energy, will be particularly dramatic in the case if fluorinated fragments are generated during the baking process of the alignment layer.

### EXAMPLE A

A 4 wgt % solution composed of the alignment material (A) in a 50:50 mixture of NMP/BC was stirred for 15 min at RT and filtrated with a 0.45 μm filter.

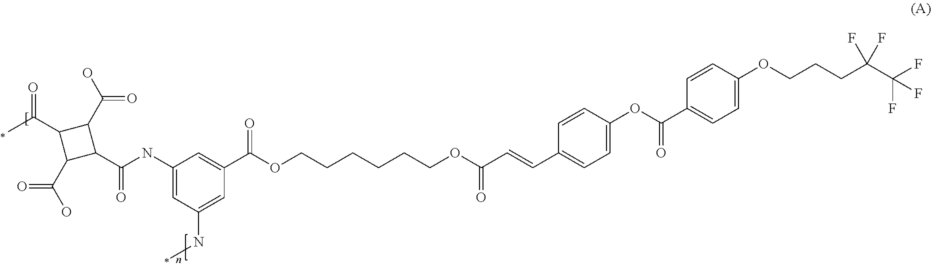

The solution was spin-coated at 1600 rpm onto an carefully cleaned ITO coated glass plate (Nemapearl X-0088-Glass-I with ITO, Nippo Denki) for 60 s and annealed at 80° C. for 1 min (layer thickness ca. 70 nm). A similar substrate (Nemapearl X-0088-Glass-I with ITO), simulating the uncoated areas, was faced to the first coated substrate at a distance of 0.7 mm (no direct contact between the two samples). The coated sample was then placed on a hot plate at 200° C. for 40 min. At the end of the baking procedure, the top layer was carefully removed and its surface energy was evaluated using the Owens-Wendt-Kaelble method.

The surface energy for the reference substrate is 65.8 mN/m. The surface energy for the contaminated top substrate is 57.0 mN/m. In this case, the reduction of the surface energy of the “uncoated areas” is less than 10 mN/m and the surface properties of the “uncoated areas” are almost not changed during the baking process.

The following table illustrate the influence of chemical structure of the material on the contamination effect measured on the top substrate.

The examples given in the next table were performed according to the experimental conditions described in Example A.

| | Surface |
|---|---|
| | energy |
| Chemical structure of the alignment polymer | (mN/m) |
| Reference substrate | 65.8 |
| 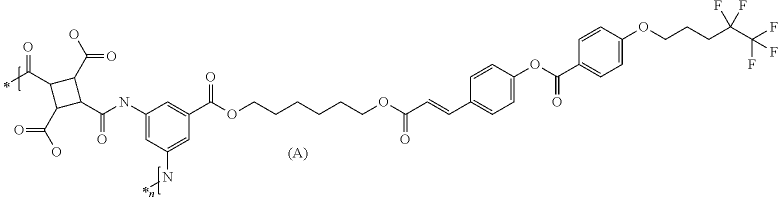 | 57.0 |
| 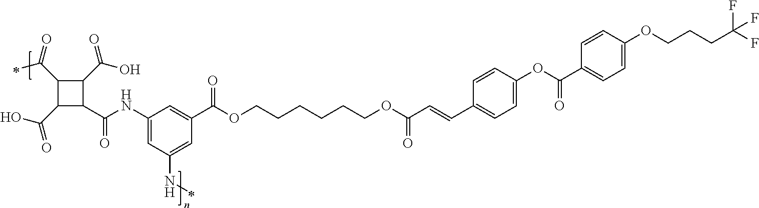 | 53.0 |
| 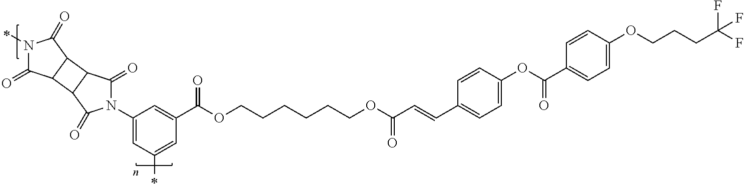 | 48.9 |
| 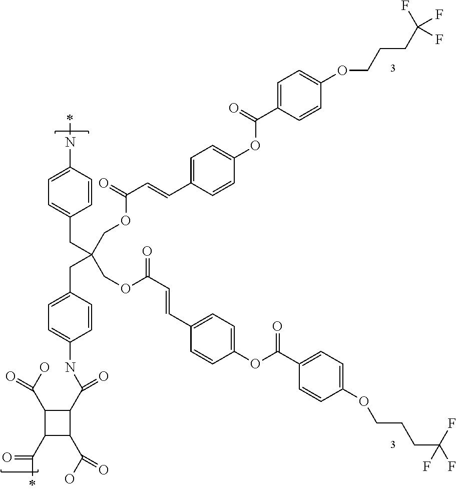 | 55.3 |
| 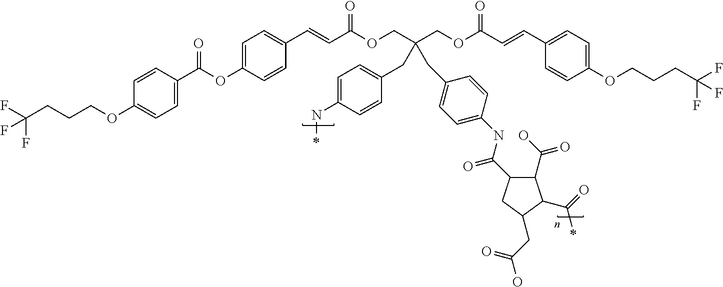 | 51.1 |
| 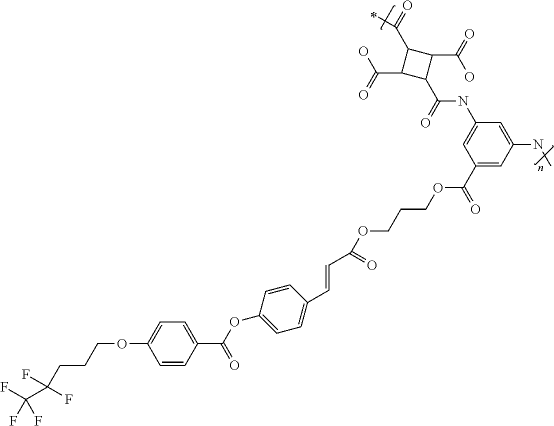 | 52.6 |
| 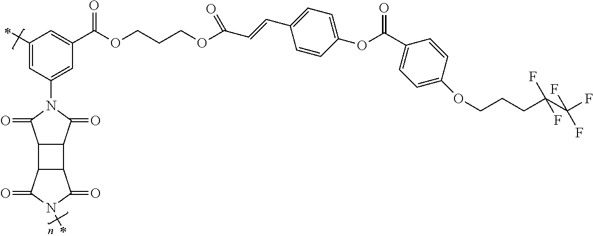 | 51.83 |
| 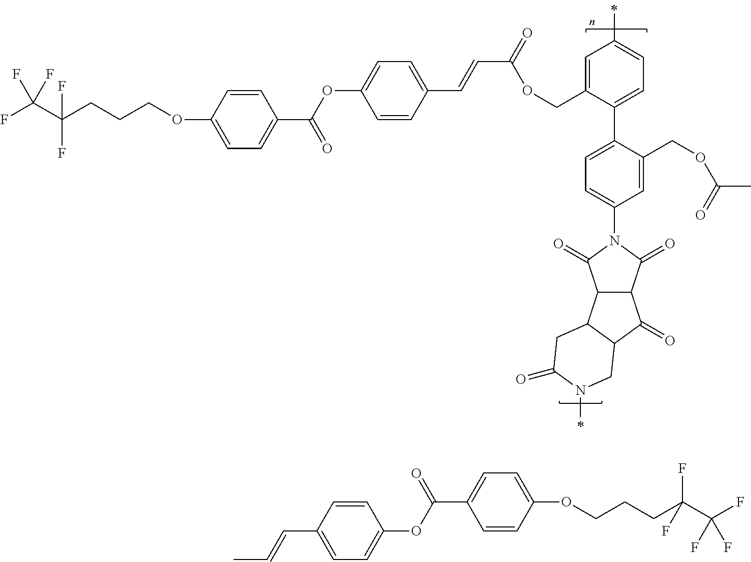 | 37.9 |

### EXAMPLES B

The next examples illustrate the influence of the length of the spacer positioned located between the backbone and the chromophore moiety for 1,2,4-substituted diamines.

The examples given in the next table were performed according to the experimental conditions described in Example A.

| | Surface |
|---|---|
| | energy |
| Chemical structure of the alignment polymer | (mN/m) |
| Comparative example | 22.6 |
| 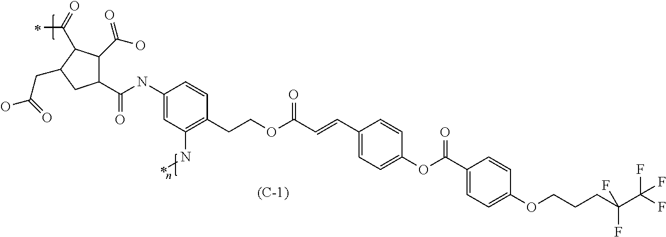 | |
| (C2) | |
| 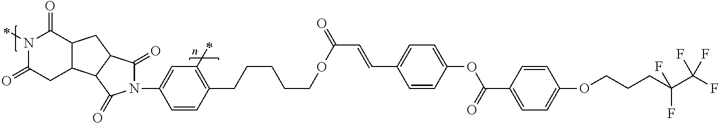 | 39.8 |
| (C5) | |
| 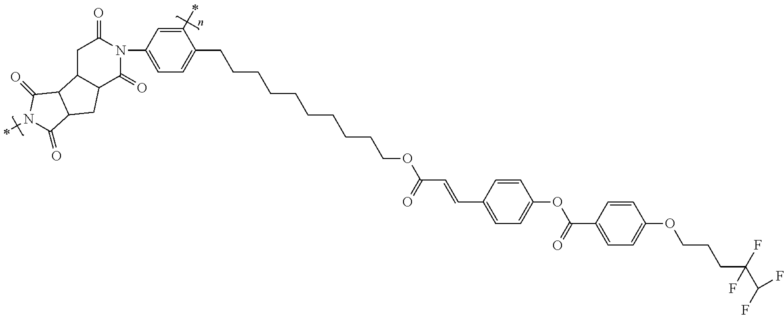 | 55.7 |
| (C10) | |

### EXAMPLES C

As shown in the nest table, the extent of the contamination (i.e. the modification of the surface energy) strongly depends on the nature of the substrate. This table points out that the changes in the surface energy due to thermal decomposition of the material are greater for ITO-coated glass plates than for Si wafers.

The examples given in the next table were performed according to the experimental conditions described in Example A with ITO-coated glass plates and Si wafers.

| | | Surface |
|---|---|---|
| | Subs- | energy |
| Chemical structure of the alignment polymer | trate | (mN/m) |
| Reference substrate | Si | 70.8 |
| | wafer | |
| Comparative example | Si | 56.7 |
| | wafer | |
| 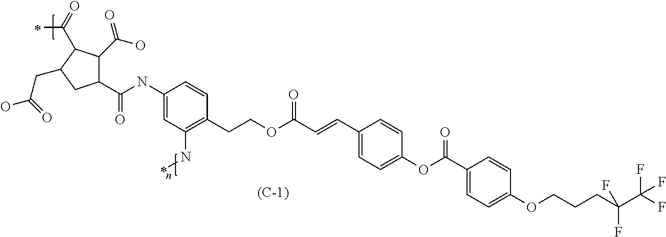 | | |
| Reference substrate | ITO | 65.8 |
| Comparative example | ITO | 22.6 |
| 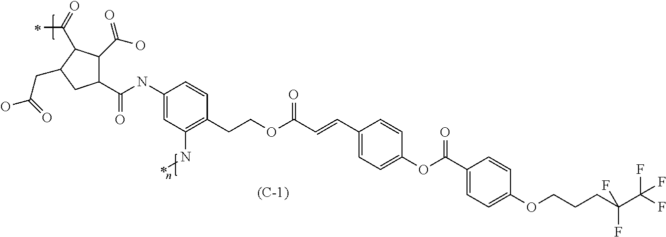 | | |

### EXAMPLES E

The next examples point out that the moiety positioned at the end of the side chain does not or only slightly influences the thermal stability of the alignment material i.e. the extent of the contamination.

The examples given in the next table were performed according to the experimental conditions described in Example A.

| | Surface |
|---|---|
| | energy |
| Chemical structure of the alignment polymer | (mN/m) |
| 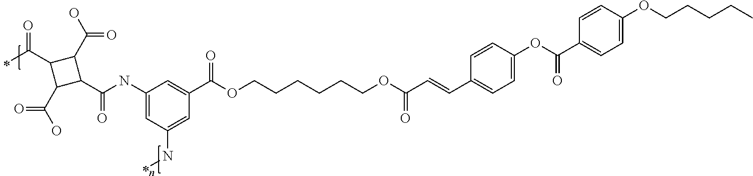 | 62.2 |
| 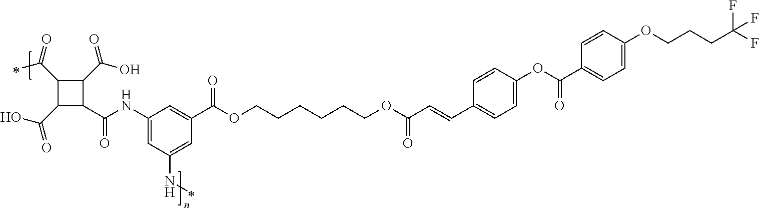 | 53.0 |
| 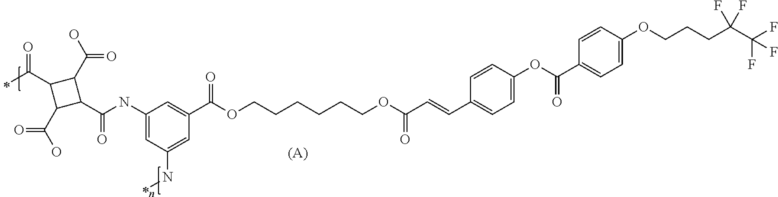 | 57.0 |

1. Use of thermally stable alignment materials for the preparation of orientation layers for liquid crystals comprising diamine compounds of diamine compounds of formula (I′), 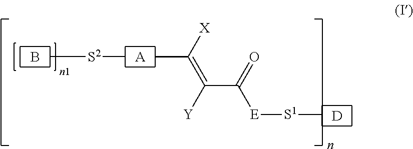
wherein,
A represents an unsubstituted or substituted carbocyclic or heterocyclic aromatic group selected from a monocyclic ring of five or six atoms, two adjacent monocyclic rings of five or six atoms, a bicyclic ring system of eight, nine or ten atoms, or a tricyclic ring system of thirteen or fourteen atoms; and
wherein
B represents a straight-chain or branched C₁-C₁₆alkyl group, which is unsubstituted or substituted by
di-(C₁-C₁₆alkyl)amino, C₁-C₆alkyloxy, nitro, cyano, and/or halogene, such as fluorine, chlorine, bromine or jodine; and wherein one or more —CH₂— group may independently from each other be replaced by a linking group;
D is a selected from the group of the following compounds: 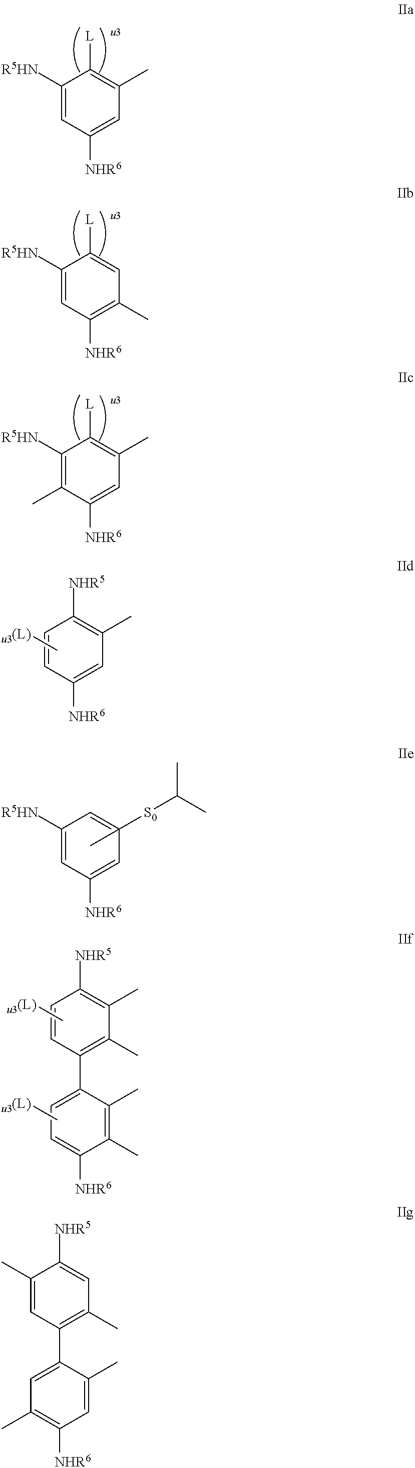 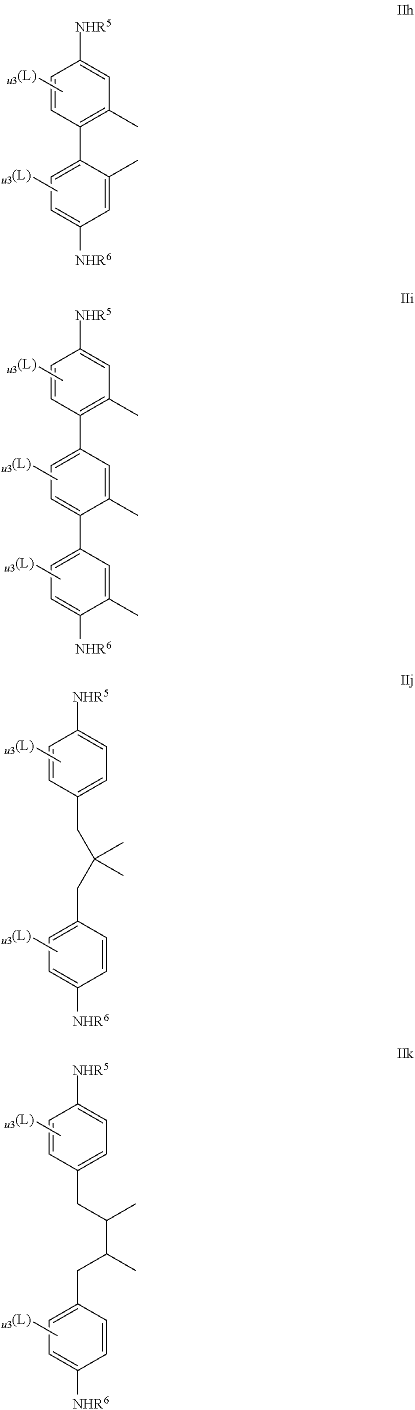 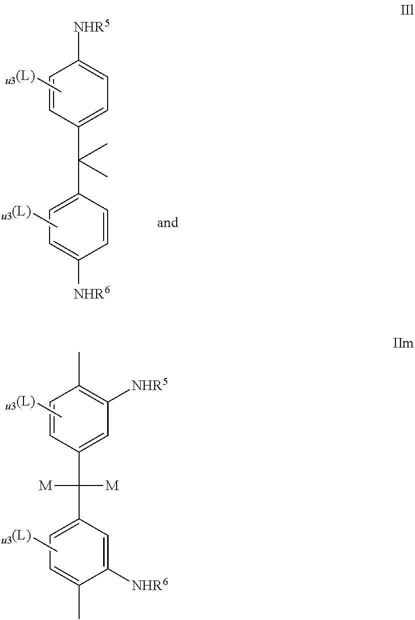
“-” denotes the linking(s) of D to S¹ in compound (I) and represents a single bond; and
S⁰ represents a single bond or a spacer unit, which is a straight-chain or branched, substituted or unsubstituted C₁-C₂₄alkylen, preferably C₁-C₆alkylen, wherein one or more, preferably non-adjacent, —CH₂— group may be replaced by a linking group, preferably replaced by —O—, —O(CO)—, —(CO)O—, —NR₁CO—, —CONR₁—, wherein R₁ is hydrogen or C₁-C₆alkyl; most preferably S⁰ is a single bond;
M represents a H, C₁-C₂₄alkyl and CF₃;
L is —CH₃, —COCH₃, —OCH₃, nitro, cyano, halogen, CH₂═CH—, CH₂═C(CH₃)—, CH₂═CH—(CO)O—, CH₂═CH—O—, —NR⁵R⁶, CH₂═C(CH₃)—(CO)O— or CH₂═C(CH₃)—O—,
wherein:
R⁵, R⁶ each independently from each other represents a hydrogen atom or C₁-C₆alkyl;
u₃ is an integer from 0 to 2;
and/or an alicyclic group;
E represents an aromatic group, an oxygen atom, a sulphur atom, —NH—, —N(C₁-C₆alkyl)-, —CR²R³,
wherein R² and R³ are independently from each other hydrogen or a cyclic, straight-chain or branched, substituted or unsubstituted C₁-C₂₄alkyl, wherein one or more —CH₂— group(s) may be independently from each other replaced by a linking group, and with the proviso that at least one of R² and R³ is not hydrogen;
S¹ represents a single bond or a cyclic, straight-chain or branched, substituted or unsubstituted C₁-C₂₄alkylen, if D is a compound of formulae IIa, IIc, IId, IIe, IIf, IIg, IIh, IIi, IIj, IIk; and if D is a compound of formula IIb, S¹ represents a single bond or a cyclic, straight-chain or branched, substituted or unsubstituted C₂-C₂₄alkylen;
S² represents a spacer unit;
X, Y each independently from each other represents hydrogen, fluorine, chlorine, cyano, unsubstituted or with fluorine substituted C₁-C₁₂alkyl, in which one or more —CH₂— groups may be replaced by a linking group;
n, n1 each independently from each other represents 1, 2, 3 or 4;
with the proviso that if n is 2, 3, or 4, each A, B, x₁, E, S¹, S², X, Y are identical or different; and if n1 is 2, 3 or 4 each B, x₁ is identical or different.

2. Use according to claim 1, wherein the diamine is a compound of formulae (VI), (VII), (VIII), (IX), (X), (XI), (XIa) and (XIb) 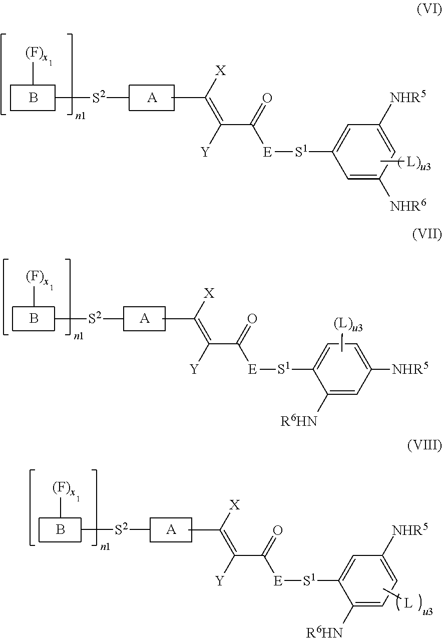
wherein in (VII) and (VIII) S¹ represents a single bond or a cyclic, straight-chain or branched, substituted or unsubstituted C₂-C₂₄alkylen, preferably C₅-C₂₄alkylen, more preferably C₁₀-C₂₄alkylen; the other substituents have the same meaning as given below for (VI), (IX), (X), (XI), (XIa), (XIb), (XIc) and (XId) 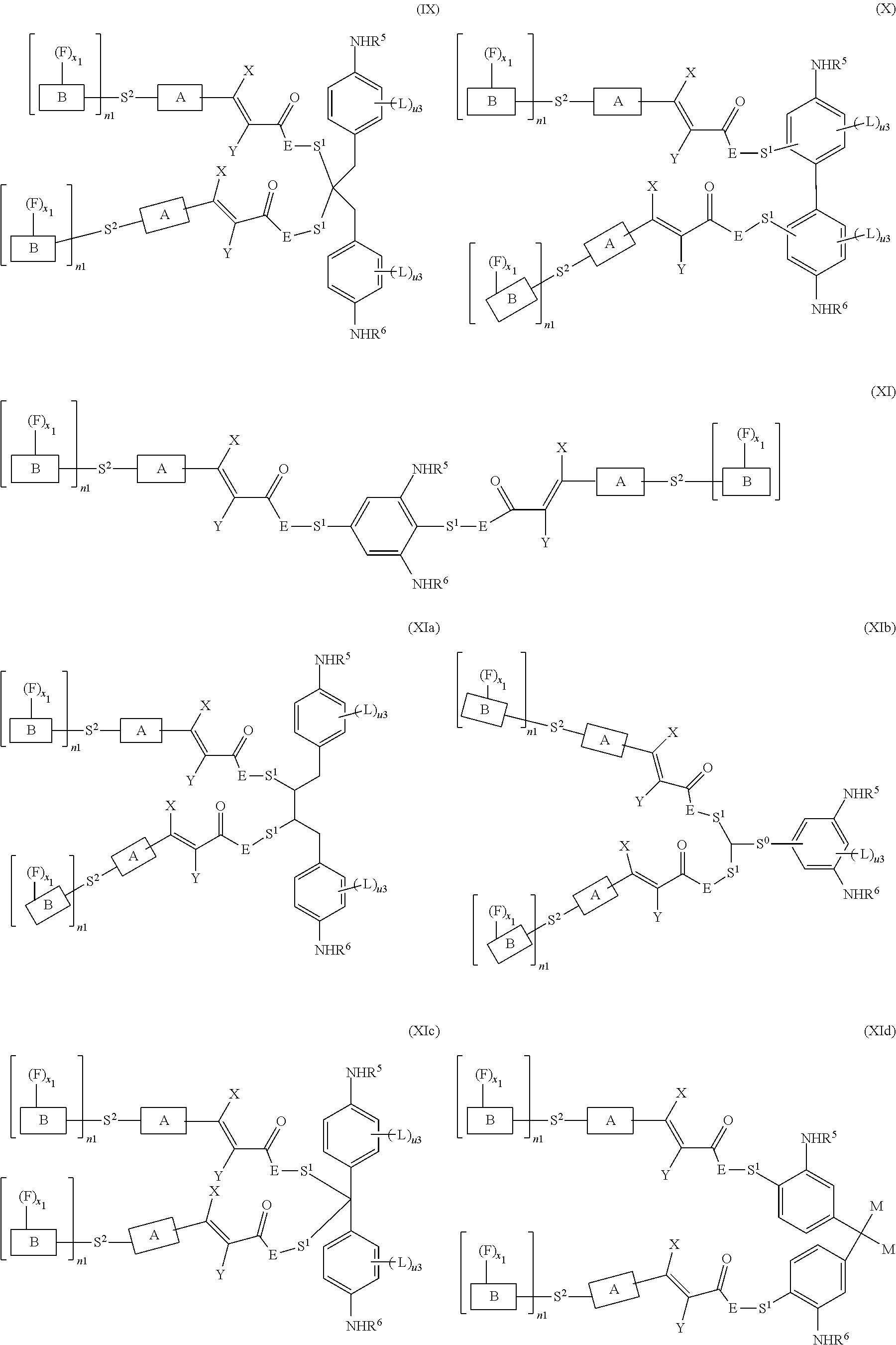
wherein
A, B, x₁, n, n1, D, E, M, S², S¹, S⁰, X and Y have the above given meanings as in claim 1 and R⁵, R⁶ have the same meanings as in claim 1;
L is —CH₃, —OCH₃, —COCH₃, nitro, cyano, halogen, CH₂═CH—, CH₂═C(CH₃)—, CH₂═CH—(CO)O—, CH₂═CH—O—, CH₂═C(CH₃)—(CO)O—, or CH₂═C(CH₃)—O—,
u₃ is an integer from 0 to 2.

3. Use according to claim 1, wherein the orientation layer comprises coated and uncoated areas, whereby the surface of the uncoated areas is not contaminated during the thermal baking in the process of preparation of an orientation layer.

4. Use according to claim 1, wherein the uncoated areas have a surface energy of >40 mN/Meter on ITO coated glass plates.

5. Polymer, copolymer or oligomer comprising diamine (I′) according to claim 1, as one basic building block.

6. Polymer, copolymer or oligomer according to claim 5, which is a polyamic acid, polyamic ester, polyimide or a mixture of polyamic acid and polyamic ester and/or polyimide.

7. Composition comprising at least one diamine (I′) according to claim 1 and optionally at least one further diamine, which is different from (I′) or/and an additive.

8. Process for the preparation of a polymer, copolymer or oligomer comprising polymerisation of a diamine (I′) according to claim 1.

9. Polymer, copolymer or oligomer comprising a diamine of compound (I′) according to claim 1.

10. Polymer, copolymer or oligomer according to claim 5 or prepared according to claim 8, which is a polymer gel or a polymer network, copolymer gel or a copolymer network, or an oligomer gel or an oligomer network.

11. Polymer, copolymer or oligomer according to any of claim 5 or 9 or prepared according to claim 8, which is able to undergo photocyclization.

12. Composition, especially a blend, comprising
a polymer, copolymer or oligomer comprising at least a diamine (I′) according to claim 1 as basic building block, or
a polymer, copolymer or oligomer obtainable according to claim 8.

13. Method for the preparation of a polymer, copolymer or oligomer according to any of claim 5 or 9 or prepared according to claim 8, wherein in a polycondensation reaction a diamine (I′) according to claim 1 is reacted with one or more tetracarboxylic acid anhydride, optionally in the presence of one or more additional other diamines.

14. Polymer, copolymer or oligomer layer, comprising at least one polymer, copolymer or oligomer according to any of claim 5 or 9 or prepared according to claim 8, preferably on a Si wafer.

15. Method for the preparation of a polymer layer or oligomer layer, wherein one or more polymers, copolymers or oligomers according to any of claim 5 or 9 or prepared according to claim 8 is applied to a support, and wherein, the polymer or oligomer or polymer mixture or oligomer mixture is treated with aligning light.

16. Polymer, copolymer or oligomer layer obtainable by the method according to claim 9.

17. Optical and electro-optical unstructured or structured constructional elements, preferably liquid crystal display cells, multi-layer and hybrid layer elements, comprising at least one polymer layer, copolymer or oligomer layer according to claim 16.

18. Orientation layer, comprising at least one polymer layer, copolymer or oligomer layer according to claim 16.
